(19) United States
(12) Patent Application Publication
(10) Pub. No.: US 2025/0011802 A1
(43) Pub. Date: Jan. 9, 2025

(54) Methods for the Modification of Cells, Modified Cells and Uses Thereof

(71) Applicant: Agriculture Victoria Services Pty Ltd, Bundoora, Victoria (AU)

(72) Inventors: Lennon James Matchett-Oates, Preston, Victoria (AU); Noel Cogan, Macleod, Victoria (AU); German Carlos Spangenberg, Bundoora, Victoria (AU); Ehab Ibrahim Ahmed Mohamaden, Williamstown, Victoria (AU)

(21) Appl. No.: 18/690,172

(22) PCT Filed: Sep. 9, 2022
(86) PCT No.: PCT/AU2022/051089

§ 371 Date: Mar. 7, 2024

(30) Foreign Application Priority Data
Sep. 9, 2021 (AU) 2021902919
Dec. 24, 2021 (AU) 2021904262

(51) Int. Cl. C12N 15/82; C12N 9/02; C12N 15/113

(52) U.S. Cl. CPC C12N 15/8243; C12N 9/0004; C12N 15/1137; C12N 15/8218; C12N 2310/14; C12Y 121/03007; C12Y 121/03008

(57) ABSTRACT

The present disclosure relates generally to methods for the modification of plant cells, e.g., cannabis plant cells, using RNA interference (RNAi), and cells that have been modified to express mediators of RNAi, e.g., short interfering RNA (siRNA). The present disclosure further relates to plants comprising the modified cells and products derived from the plants, e.g., plant material and extracts derived therefrom and methods for selecting siRNA for altering the expression of one or more cannabinoid biosynthesis genes, such as THCAS, CBCAS or CBDAS.

### RELATED APPLICATIONS

This application claims priority from Australian Provisional Application No. 2021902919 filed on 9 Sep. 2021 and Australian Provisional Application No. 2021904262 filed on 24 Dec. 2021, the entire contents of which are hereby incorporated by reference.

### FIELD

The present disclosure relates generally to methods for the modification of plant cells, e.g., cannabis plant cells, using RNA interference (RNAi), and cells that have been modified to express mediators of RNAi, e.g., short interfering RNA (siRNA). The present disclosure further relates to plants comprising the modified cells and products derived from the plants, e.g., plant material and extracts derived therefrom and methods for selecting siRNA for altering the expression of one or more cannabinoid biosynthesis genes.

### BACKGROUND

Cannabis sativa L. is one of the earliest domesticated and cultivated plants with records of its use in central Asia dating back more than 6000 years. Cannabis belongs to the Cannabaceae family and has been used for millennia for its source of fibre, seed oil, food and medicinal purposes.

The chemical profile of cannabis plants is varied. It is estimated that cannabis plants produce more than 400 different molecules, including cannabinoids, terpenes and other phenolics. Cannabinoids, such as Δ⁹-tetrahydrocannabinol (THC) and cannabidiol (CBD), are the most commonly known and researched cannabinoids. In cannabis plants, CBD and THC are naturally present in their acidic forms, Δ⁹-tetrahydrocannabinolic acid (THCA) and cannabidiolic acid (CBDA), which are alternative products of a shared precursor cannabigerolic acid (CBGA). Other cannabinoids of interest include cannabigerol (CBG), cannabichromene (CBC), cannabinol (CBN) and tetrahydrocannabivarin (THCV).

Many cannabinoids interact with the endocannabinoid system in mammals, including humans, to exert complex biological effects on the neuronal, metabolic, immune and reproductive systems. They also interact with G protein-coupled receptors (GPCRs), such as CB1 and CB2, in the human endocannabinoid system, where they are thought to play a part in the regulation of appetite, pain, mood, memory, inflammation and insulin sensitivity. Cannabinoids have also been implicated in neuronal signaling, gastrointestinal inflammation, tumorigenesis, microbial infection and diabetes. As a result, medicinal cannabis has been investigated as a potential treatment for a number of conditions, including pain (Campbell et al., 2001, BMJ, 323(7303): 13), cancer (Machado Rocha et al., 2008, European Journal of Cancer Care, 17(5): 431-443), multiple sclerosis (Rog et al., 2005, Neurology, 65(6): 812-819) and epilepsy (Russo, 2017, Epilepsy & Behaviour, 70: 292-297).

Development of new cannabis strains for medicinal purposes through traditional breeding efforts is a lengthy and expensive process, particularly in view of the complex genetic background of existing cannabis varieties. The dioecious, wind pollination nature of cannabis has created a highly diverse genetic pool in which many varieties have been generated in clandestine breeding efforts, creating a highly diverse population with high levels of sequence and copy number variations affecting the drug content (Weiblen et al., 2015, New Phytologist, 208(4): 1241-1250). This genetic complexity makes the generation of transgenic or mutant cannabis strains difficult, as the generation of stably transformed lines is known to be a lengthy process, requiring protocol development for transformation and regeneration. These difficulties are further exacerbated in the context of cannabis, as several studies have indicated a high level of recalcitrance of in vitro shoot regeneration from different tissues, including cotyledons, epicotyls, leaves and hypocotyls (see, e.g., Movahedi et al., 2015, Journal of Plant Molecular Breeding, 3: 20-27; Movahedi et al., 2016, Iranian Journal of Medicinal and Aromatic Plants Research, 32: 758-769; Smykalova et al., 2019, Plant Cell, Tissue and Organ Culture, 139: 381-394), which limits the application of in vitro tissue culture methods for the improvement of cannabis strains.

There remains, therefore, a need for improved methods for the development of new cannabis varieties for medicinal purposes.

### SUMMARY

In an aspect of the present disclosure there is provided a cannabis cell comprising a targeted genetic modification that alters the expression of one or more cannabinoid biosynthesis genes, wherein the genetic modification is targeted to one or more, or all of
a. a region of at least one CBDAS gene comprising a nucleotide sequence corresponding to any one of SEQ ID NOs: 18-26, or a or a nucleotide sequence which is at least 90% identical to the nucleotide sequence of SEQ ID NOs: 18-26;
b. a region of at least one CBCAS gene comprising a nucleotide sequence corresponding to any one of SEQ ID NOs: 15-17, or a or a nucleotide sequence which is at least 90% identical to the nucleotide sequence of SEQ ID NOs: 15-17; and
c. a region of a THCAS gene comprising a nucleotide sequence corresponding to SEQ ID NO: 14, or a or a nucleotide sequence which is at least 90% identical to the nucleotide sequence of SEQ ID NO: 14;
wherein the cell is characterized by altered expression of the one or more, or all of the cannabinoid biosynthesis genes relative to a cannabis cell that does not comprise the genetic modification.

In another aspect, the present disclosure provides a cannabis plant comprising the cell described herein.

In another aspect, the present disclosure provides a plant extract obtained from the cannabis plant described herein.

In another aspect, the present disclosure provides a synthetic short interfering RNA (siRNA) molecule comprising a nucleotide sequence selected from SEQ ID NOs: 27-322, or a nucleotide sequence which is at least 90% identical to the nucleotide sequence of SEQ ID NOs: 27-322.

In another aspect, the present disclosure provides a vector comprising the nucleic acid molecule encoding the synthetic siRNA molecule described herein.

In certain embodiments contemplated herein, the synthetic siRNA molecule or the vector is in a cell, preferably in a cannabis cells, or in plant material or extracts derived therefrom.

In another aspect, the present disclosure provides a method for producing a cannabis plant with an altered level or activity, or both, of a cannabinoid biosynthesis pathway, the method comprising:
a. introducing a targeted genetic modification to a cannabis cell leading to altered expression of one or more cannabinoid biosynthesis genes, wherein the genetic modification is targeted to one or more or all of:
   i. a region of at least one CBDAS gene comprising a nucleotide sequence corresponding to any one of SEQ ID NOs: 18-26, or a or a nucleotide sequence which is at least 90% identical to the nucleotide sequence of SEQ ID NOs: 18-26;
   ii. a region of at least one CBCAS gene comprising a nucleotide sequence corresponding to any one of SEQ ID NOs: 15-17, or a or a nucleotide sequence which is at least 90% identical to the nucleotide sequence of SEQ ID NOs: 15-17; and
   iii. a region of a THCAS gene comprising a nucleotide sequence corresponding to SEQ ID NO: 14, or a or a nucleotide sequence which is at least 90% identical to the nucleotide sequence of SEQ ID NO: 14; and
b. regenerating a cannabis plant from the cell of step (a), wherein the plant comprises the genetic modification and has an altered level or activity, or both, of the cannabinoid biosynthesis pathway relative to a cannabis plant that does not comprise the genetic modification.

In another aspect, the present disclosure provides a method for selecting a siRNA for altering the expression of one or more cannabinoid biosynthesis genes, the method comprising:
a. providing a set of potential siRNA which target one or more or all of a THCAS gene, at least one CBDAS gene and at least one CBCAS gene;
b. determining the binding complementarity of the siRNA to:
   i. a region of at least one CBDAS gene;
   ii. a region of at least one CBCAS gene; and
   iii. a region of a THCAS gene; and
c. selecting an siRNA that has on-target binding complementarity to regions (i)-(iii) and negligible off-target binding complementarity to a region outside of regions (i)-(iii).

In another aspect, the present disclosure provides a method of altering the expression of cannabinoid biosynthesis genes in a cannabis cell, the method comprising transiently transfecting a cannabis cell with an RNA interference (RNAi) construct comprising a nucleotide sequence encoding a siRNA via agroinfiltration, wherein the siRNA targets one or more or all of a THCAS gene, at least one CBDAS gene and at least one CBCAS gene.

In another aspect, the present disclosure provides a method of altering the expression of cannabinoid biosynthesis genes in a cannabis cell, the method comprising stably transducing a cannabis cell with an RNAi vector comprising a nucleic acid sequence encoding a siRNA, wherein the siRNA targets one or more or all of a THCAS gene, at least one CBDAS gene and at least one CBCAS gene.

In another aspect, the present disclosure provides a transgenic cannabis plant comprising an RNAi vector comprising a nucleotide sequence encoding a siRNA, wherein the siRNA targets one or more or all of a THCAS gene, at least one CBDAS gene and at least one CBCAS gene.

### BRIEF DESCRIPTION OF THE DRAWINGS

Embodiments of the disclosure are described herein, by way of non-limiting example only, with reference to the accompanying drawings.

FIG. 1 is a schematic representation of the phylogenetic tree of coding sequence data from cannabinoid biosynthesis genes in the exemplified cannabis plant demonstrating the highly homologous nature of gene homologs.

FIG. 2 is a graphical representation of the gene alignments used for PCR amplification for siRNA generation.

FIG. 3 shows the cannabis agroinfiltration process. (A) A photographic representation of a 2-month old donor cannabis plant; (B) A photographic representation of the cannabis leaf disk used in agroinfiltration, scale bar=2 cm; (C) A photographic representation of leaf disk in agroinfiltration media prior to vacuum infiltration, scale bar=2 cm; and (D) A photographic representation of vacuum infiltration of leaf discs.

FIG. 4 shows the effect of different pRNAi-GG vectors on cannabinoid biosynthesis gene relative expression. A graphical representation of relative fold change (y-axis) of cannabinoid biosynthesis genes post-agroinfiltration with (A) pRNAi-GG-THCAS; (B) pRNAi-GG-CBDAS; (C) pRNAi-GG-CBCAS; and (D) pRNAi-GG-CBDAS-UNIVERSAL. Significance was determined by paired t-test, p<0.05 is denoted by *. Error bars represent SE.

FIG. 5 is a schematic representation of the pRNAi-GG-THCAS vector.

FIG. 6 is a schematic representation of the pRNAi-GG-CBDAS vector.

FIG. 7 is a schematic representation of the pRNAi-GG-CBCAS vector.

FIG. 8 is a schematic representation of the pRNAi-GG-CBDAS-UNIVERSAL vector.

FIG. 9 is a photographic representation of cotyledon callus formation on callus induction media (CIM) measured using ImageJ.

FIG. 10 shows that transformed cotyledons out-grow control cotyledons. (A) A graphical representation of callus size (cm²; y-axis) and time (weeks on CIM; x-axis). (B) A graphical representation of callus weight (g; y-axis) and time (weeks on CIM; x-axis). Control is right hand bar and transformed is left hand bar. Error bars are representative of standard deviation. *p<0.05.

FIG. 11 shows callus response to different regeneration treatments and transformation frequency. (A) A photographic representation of callus response to 2.5 μM TDZ treatment. (B) A photographic representation of callus response to 5 μM TDZ treatment. (C) A photographic representation of callus response to 10 μM TDZ treatment. (D) A photographic representation of control callus.

FIG. 12 shows the regeneration of control callus. (A) A photographic representation of regenerating shoots from calli on 5 μM TDZ regeneration media. (B) A photographic representation of regenerating shoots from calli on 10 μM TDZ regeneration media. (C) A photographic representation of regenerated shoot in rooting media at 7 days post-transfer from CIM. (D) A photographic representation of regenerated shoot in rooting media at 28 days post-transfer from CIM.

FIG. 13 is a series of photographic representations of the transformation efficiency of GFP-expressing calli after 5 weeks growth on regeneration media.

FIG. 14 shows the spontaneous rooting of regenerating transformed hypocotyls. (A) A photographic representation of spontaneous rooting of co-transformed hypocotyls. (B) A photographic representation of spontaneous rooting of control hypocotyls (transformed with disarmed Agrobacterium). Arrows indicating location of rooting activity.

FIG. 15 is a photographic representation of regenerated (A) control hypocotyl and (B) transformed hypocotyl.

FIG. 16 is a series of photographic representations of GFP-expressing co-transformed hypocotyls after 4 weeks growth on regeneration media.

FIG. 17 is a photographic representation of a GFP-expressing regenerated transformed hypocotyl leaf.

FIG. 18 is a graphical representation of relative expression levels of THCAS (left hand bar), CBDAS (middle bar), and CBCAS (right hand bar) (y-axis) in transformed callus (x-axis). *p<0.05, **p<0.01, ***p<0.001.

FIG. 19 is a schematic representation of the pDPI000013 GFP encoding vector.

### BRIEF DESCRIPTION OF THE SEQUENCES

Nucleic acid sequences are referred to by a sequence identifier number (SEQ ID NO), with reference to the accompanying sequence listing.

SEQ ID NO: 1 shows the genomic nucleotide sequence of THCAS.

SEQ ID NO: 2 shows the genomic nucleotide sequence of CBCAS-1.

SEQ ID NO: 3 shows the genomic nucleotide sequence of CBCAS-2.

SEQ ID NO: 4 shows the genomic nucleotide sequence of CBCAS-truncated.

SEQ ID NO: 5 shows the genomic nucleotide sequence of CBDAS-1.

SEQ ID NO: 6 shows the genomic nucleotide sequence of CBDAS-2.

SEQ ID NO: 7 shows the genomic nucleotide sequence of CBDAS-like-1.

SEQ ID NO: 8 shows the genomic nucleotide sequence of CBDAS-like-2.

SEQ ID NO: 9 shows the genomic nucleotide sequence of CBDAS-like-3.

SEQ ID NO: 10 shows the genomic nucleotide sequence of CBDAS-truncated-1.

SEQ ID NO: 11 shows the genomic nucleotide sequence of CBDAS-truncated-2.

SEQ ID NO: 12 shows the genomic nucleotide sequence of CBDAS-truncated-3.

SEQ ID NO: 13 shows the genomic nucleotide sequence of CBDAS-truncated-4.

SEQ ID NO: 14 shows the shows the coding/exon nucleotide sequence of THCAS.

SEQ ID NO: 15 shows the coding/exon nucleotide sequence of CBCAS-1.

SEQ ID NO: 16 shows the coding/exon nucleotide sequence of CBCAS-2.

SEQ ID NO: 17 shows the coding/exon nucleotide sequence of CBCAS-truncated.

SEQ ID NO: 18 shows the coding/exon nucleotide sequence of CBDAS-1.

SEQ ID NO: 19 shows the coding/exon nucleotide sequence of CBDAS-2.

SEQ ID NO: 20 shows the coding/exon nucleotide sequence of CBDAS-like-1.

SEQ ID NO: 21 shows the coding/exon nucleotide sequence of CBDAS-like-2.

SEQ ID NO: 22 shows the coding/exon nucleotide sequence of CBDAS-like-3.

SEQ ID NO: 23 shows the coding/exon nucleotide sequence of CBDAS-truncated-1.

SEQ ID NO: 24 shows the coding/exon nucleotide sequence of CBDAS-truncated-2.

SEQ ID NO: 25 shows the coding/exon nucleotide sequence of CBDAS-truncated-3.

SEQ ID NO: 26 shows the coding/exon nucleotide sequence of CBDAS-truncated-4.

SEQ ID NO: 27-119 show the siRNAs generated for the coding/exon nucleotide sequence of THCAS as shown in SEQ ID NO: 14.

SEQ ID NO: 120-189 show the siRNAs generated for the coding/exon nucleotide sequence of CBDAS-like-1 as shown in SEQ ID NO: 20.

SEQ ID NO: 190-284 show the siRNAs generated for the coding/exon nucleotide sequence of CBCAS-2 as shown in SEQ ID NO: 16.

SEQ ID NO: 285-322 show the siRNAs generated for the coding/exon nucleotide sequence of CBDAS-truncated-4 as shown in SEQ ID NO: 13.

SEQ ID NO: 323 shows the forward primer sequence for cDNA amplification of THCAS.

SEQ ID NO: 324 shows the reverse primer sequence for cDNA amplification of THCAS.

SEQ ID NO: 325 shows the forward primer sequence for cDNA amplification of CBDAS-like-1.

SEQ ID NO: 326 shows the reverse primer sequence for cDNA amplification of CBDAS-like-1.

SEQ ID NO: 327 shows the forward primer sequence for cDNA amplification of CBCAS-2.

SEQ ID NO: 328 shows the reverse primer sequence for cDNA amplification of CBCAS-2.

SEQ ID NO: 329 shows the forward primer sequence for cDNA amplification of CBDAS-truncated-4.

SEQ ID NO: 330 shows the reverse primer sequence for cDNA amplification of CBDAS-truncated-4.

SEQ ID NO: 331 shows the forward primer sequence for Flanking Arm #1 for recombinant E. coli.

SEQ ID NO: 332 shows the reverse primer sequence for Flanking Arm #1 for recombinant E. coli.

SEQ ID NO: 333 shows the forward primer sequence for Flanking Arm #2 for recombinant E. coli.

SEQ ID NO: 334 shows the reverse primer sequence for Flanking Arm #2 for recombinant E. coli.

SEQ ID NO: 335 shows the THCAS amplified cDNA insert comprised in pRNAi-GG-THCAS.

SEQ ID NO: 336 shows the CBDAS-like-1 amplified cDNA insert comprised in pRNAi-GG-CBDAS.

SEQ ID NO: 337 shows the CBCAS-2 amplified cDNA insert comprised in pRNAi-GG-CBCAS.

SEQ ID NO: 338 shows the CBDAS-truncated-4 amplified cDNA insert comprised in pRNAi-GG-CBDAS-UNIVERSAL.

SEQ ID NO: 339 shows the forward primer sequence for qPCR amplification of THCAS.

SEQ ID NO: 340 shows the reverse primer sequence for qPCR amplification of THCAS.

SEQ ID NO: 341 shows the forward primer sequence for qPCR amplification of CBDAS #1.

SEQ ID NO: 342 shows the reverse primer sequence for qPCR amplification of CBDAS #1.

SEQ ID NO: 343 shows the forward primer sequence for qPCR amplification of CBCAS.

SEQ ID NO: 344 shows the reverse primer sequence for qPCR amplification of CBCAS.

SEQ ID NO: 345 shows the forward primer sequence for qPCR amplification of CBDAS #2.

SEQ ID NO: 346 shows the reverse primer sequence for qPCR amplification of CBDAS #2.

### DETAILED DESCRIPTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it is understood that such identifiers can change and particular information on the internet can come and go, but equivalent information can be found by searching the internet. Reference to the identifier evidences the availability and public dissemination of such information.

Unless otherwise indicated the molecular biology, cell culture, laboratory, plant breeding and selection techniques utilized in the present invention are standard procedures, well known to those skilled in the art. Such techniques are described and explained throughout the literature in sources such as, Perbal, A Practical Guide to Molecular Cloning, John Wiley and Sons (1984), Sambrook et al., Molecular Cloning: A Laboratory Manual, Cold Spring Harbor Laboratory Press (1989), Brown (editor), Essential Molecular Biology: A Practical Approach, Volumes 1 and 2, IRL Press (1991), Glover and Hames (editors), DNA Cloning: A Practical Approach, Volumes 1-4, IRL Press (1995 and 1996), Ausubel et al. (editors), Current Protocols in Molecular Biology, Greene Pub. Associates and Wiley-Interscience (1988, including all updates until present); Janick (2001) Plant Breeding Reviews, John Wiley & Sons, 252 p.; Jensen ed. (1988) Plant Breeding Methodology, John Wiley & Sons, 676 p., Richard, A. J. ed. (1990) Plant Breeding Systems, Unwin Hyman, 529 p.; Walter ed. (1987) Plant Breeding, Vol. I, Theory and Techniques, MacMillan Pub. Co.; Slavko ed. (1990) Principles and Methods of Plant Breeding, Elsevier, 386 p.; and Allard, R. W. ed. (1999) Principles of Plant Breeding, John-Wiley & Sons, 240 p. The ICAC Recorder, Vol. XV no. 2: 3-14; all of which are incorporated by reference. The procedures described are believed to be well known in the art and are provided for the convenience of the reader. All other publications mentioned in this specification are also incorporated by reference in their entirety.

The articles “a”, “an” and “the” include plural aspects unless the context clearly dictates otherwise. Thus, for example, reference to “an allele” includes a single allele, as well as two or more alleles; reference to “a treatment” includes a single treatment, as well as two or more treatments; and so forth.

In the context of this specification, the term “about” in relation to a numerical value or range is intended to cover numbers falling within +10% of the specified numerical value or range.

Each embodiment in this specification is to be applied mutatis mutandis to every other embodiment, unless expressly stated otherwise.

Genes and other genetic material (e.g., mRNA, constructs, etc.) are represented in italics and their proteinaceous expression products are represented in non-italicized form. Thus, for example, THCAS is an expression product of THCAS. Any nucleic acid sequence presented herein may be in DNA or RNA form and may be double- or single-stranded, as appropriate, though only a single strand may be provided herein (one of skill in the art will recognize that the double-stranded form comprises a complementary strand). It will be appreciated that a sequence presented in RNA form will contain “U” residues while the same sequence in DNA form would contain “T” at the positions occupied by “U” in the RNA form of the sequence. It will also be appreciated that a nucleic acid may comprise DNA, RNA, both DNA and RNA, and may contain naturally occurring nucleosides (which term includes ribonucleosides), and/or one or modified nucleosides (wherein the base, sugar, or both may be modified), modified backbones, etc., as recognized in the art.

Nucleotide and amino acid sequences are referred to by a sequence identifier number (i.e., SEQ ID NO:). The SEQ ID NOs: correspond numerically to the sequence identifiers <400>1 (SEQ ID NO: 1), <400>2 (SEQ ID NO: 2), etc. A sequence listing is provided after the claims. A list describing the SEQ ID NOs in the sequence listing is provided above under the section “Brief Description of the Sequences”.

All sequence identifiers (e.g., GenBank ID, EMBL-Bank ID, DNA Data Bank of Japan (DDBJ) ID, etc.) provided herein were current at the filing date.

Throughout this specification, unless the context requires otherwise, the word “comprise”, or variations such as “comprises” or “comprising”, will be understood to mean the inclusion of a stated element or integer or group of elements or integers but not the exclusion of any other element or integer or group of elements or integers.

The term “optionally” is used herein to mean that the subsequent described feature may or may not be present or that the subsequently described event or circumstance may or may not occur. Hence the specification will be understood to include and encompass embodiments in which the feature is present and embodiments in which the feature is not present, and embodiment in which the event or circumstance occurs as well as embodiments in which it does not.

The present invention is based in part on the surprising observations made in the experiments described herein that a cannabis cell comprising a targeted genetic modification in a region of a THCAS, CBDAS or CBCAS gene is sufficient to alter the expression of one or more, or all of the cannabinoid biosynthesis genes in the cannabis cell. In certain embodiments described herein, the cannabis cell may be used to generate a cannabis plant with an altered cannabinoid profile that is particularly useful in the generation of cannabis strains that are suitable for therapeutic use.

Accordingly, in an aspect, the present disclosure provides a cannabis cell comprising a targeted genetic modification that alters the expression of one or more cannabinoid biosynthesis genes, wherein the genetic modification is targeted to one or more, or all of:
a. a region of at least one CBDAS gene comprising a nucleotide sequence corresponding to any one of SEQ ID NOs: 18-26, or a or a nucleotide sequence which is at least 90% identical to the nucleotide sequence of SEQ ID NOs: 18-26;
b. a region of at least one CBCAS gene comprising a nucleotide sequence corresponding to any one of SEQ ID NOs: 15-17, or a or a nucleotide sequence which is at least 90% identical to the nucleotide sequence of SEQ ID NOs: 15-17; and
c. a region of a THCAS gene comprising a nucleotide sequence corresponding to SEQ ID NO: 14, or a or a nucleotide sequence which is at least 90% identical to the nucleotide sequence of SEQ ID NO: 14;
wherein the cell is characterized by altered expression of the one or more cannabinoid biosynthesis genes relative to a cannabis cell that does not comprise the genetic modification.

### Cannabis Cells

Cannabis is an erect annual herb with a dioecious breeding system, although monoecious plants exist. Wild and cultivated forms of cannabis are morphologically variable, which has resulted in difficulty defining the taxonomic organization of the genus.

As used herein, the term “cannabis” means a plant, plant part, seed or product derived therefrom of the species Cannabis sativa, Cannabis indica and Cannabis ruderalis.

In an embodiment, the cannabis cell is derived from a Cannabis spp. selected from the group consisting of Cannabis sativa, Cannabis indica and Cannabis ruderalis. In another embodiment, the cannabis cell is derived from Cannabis sativa.

The reference genome for C. sativa is the assembled draft genome and transcriptome of “Purple Kush” or “PK” (van Bakal et al. 2011, Genome Biology, 12:R102). C. sativa, has a diploid genome (2n=20) with a karyotype comprising nine autosomes and a pair of sex chromosomes (X and Y). Female plants are homogametic (XX) and males heterogametic (XY) with sex determination controlled by an X-to-autosome balance system.

The term “cannabinoid”, as used herein, refers to a family of terpeno-phenolic compounds, of which more than 100 compounds are known to exist in nature. Cannabinoids will be known to persons skilled in the art, illustrative examples of which are provided in Table 1, below, including acidic and decarboxylated forms thereof.

Cannabinoids are synthesized in cannabis plants as carboxylic acids. Acid forms of cannabinoids will be known to persons skilled in the art, illustrative examples of which are described in Papaset et al. (2018, International Journal of Medical Sciences, 15(12): 1286-1295) and Cannabis and Cannabinoids (PDQ®): Health Professional Version; PDQ Integrative, Alternative, and Complementary Therapies Editorial Board; Bethesda (MD): National Cancer Institute (US); 2002-2018).

The precursors of cannabinoids originate from two distinct biosynthetic pathways: the polyketide pathway, giving rise to olivetolic acid (OLA) and the plastidal 2-C-methyl-D-erythritol 4-phosphate (MEP) pathway, leading to the synthesis of geranyl diphosphate (GPP). OLA is formed from hexanoyl-CoA, derived from the short-chain fatty acid hexanoate, by aldol condensation with three molecules of malonyl-CoA. This reaction is catalyzed by a polyketide synthase (PKS) enzyme and an olivetolic acid cyclase (OAC). The geranylpyrophosphate:olivetolate geranyltransferase catalyzes the alkylation of OLA with GPP leading to the formation of CBGA, the central precursor of various cannabinoids. Three oxidocyclases are responsible for the diversity of cannabinoids: tetrahydrocannabinolic acid synthase (THCAS) converts CBGA to THCA, while cannabidiolic acid synthase (CBDAS) forms CBDA, and cannabichromeic acid synthase (CBCAS) produces CBCA. Propyl cannabinoids (cannabinoids with a C3 side-chain, instead of a C5 side-chain), such as tetrahydrocannabivarinic acid (THCVA), are synthetized from a divarinolic acid precursor.

As used herein, the phrase “cannabinoid biosynthesis genes” refers to genes encoding THCAS, CBDAS and CBCAS.

“Tetrahydrocannabinolic acid synthase” or “THCAS” is a monomeric oxygen dependent flavoprotein that converts CBGA into THCA.

“Δ-9-tetrahydrocannabinolic acid” or “THCA” is synthesized from the CBGA by THCAS. The neutral form “Δ-9-tetrahydrocannabinol” or “THC” is associated with psychoactive effects of cannabis, which are primarily mediated by its activation of CB1G-protein coupled receptors, which result in a decrease in the concentration of cyclic AMP (cAMP) through the inhibition of adenylate cyclase. THC also exhibits partial agonist activity at the cannabinoid receptors CB1 and CB2. CB1 is mainly associated with the central nervous system, while CB2 is expressed predominantly in the cells of the immune system. As a result, THC is also associated with pain relief, relaxation, fatigue, appetite stimulation, and alteration of the visual, auditory and olfactory senses. Furthermore, more recent studies have indicated that THC mediates an anti-cholinesterase action, which may suggest its use for the treatment of Alzheimer's disease and myasthenia (Eubanks et al., 2006, Molecular Pharmaceuticals, 3(6): 773-7).

In an embodiment, the THCAS gene comprises a nucleotide sequence corresponding to SEQ ID NO: 1, or a nucleotide sequence which is at least 90% identical to the nucleotide sequence of SEQ ID NO: 1.

“Cannabidiolic acid synthase” or “CBDAS” is a monomeric oxygen dependent flavoprotein that converts CBGA into CBDA.

“Cannabidiolic acid” or “CBDA” is also a derivative of cannabigerolic acid (CBGA), which is converted to CBDA by CBDA synthase. Its neutral form, “cannabidiol” or “CBD” has antagonist activity on agonists of the CB1 and CB2 receptors. CBD has also been shown to act as an antagonist of the putative cannabinoid receptor, GPR55. CBD is commonly associated with therapeutic or medicinal effects of cannabis and has been suggested for use as a sedative, anti-inflammatory, anti-anxiety, anti-nausea, atypical anti-psychotic, and as a cancer treatment. CBD can also increase alertness, and attenuate the memory impairing effect of THC.

In an embodiment, the at least one CBDAS gene is selected from CBDAS-1, CBDAS-2, CBDAS-like-1, CBDAS-like-2, CBDAS-like-3, CBDAS-truncated-1, CBDAS-truncated-2, CBDAS-truncated-3 and CBDAS-truncated-4.

In an embodiment, the CBDAS-1 gene comprises a nucleotide sequence corresponding to SEQ ID NO: 5, or a nucleotide sequence which is at least 90% identical to the nucleotide sequence of SEQ ID NO: 5.

In an embodiment, the CBDAS-2 gene comprises a nucleotide sequence corresponding to SEQ ID NO: 6, or a nucleotide sequence which is at least 90% identical to the nucleotide sequence of SEQ ID NO: 6.

In an embodiment, the CBDAS-like-1 gene comprises a nucleotide sequence corresponding to SEQ ID NO: 7, or a nucleotide sequence which is at least 90% identical to the nucleotide sequence of SEQ ID NO: 7.

In an embodiment, the CBDAS-like-2 gene comprises a nucleotide sequence corresponding to SEQ ID NO: 8, or a nucleotide sequence which is at least 90% identical to the nucleotide sequence of SEQ ID NO: 8.

In an embodiment, the CBDAS-like-3 gene comprises a nucleotide sequence corresponding to SEQ ID NO: 9, or a nucleotide sequence which is at least 90% identical to the nucleotide sequence of SEQ ID NO: 9.

In an embodiment, the CBDAS-truncated-1 gene comprises a nucleotide sequence corresponding to SEQ ID NO: 10, or a nucleotide sequence which is at least 90% identical to the nucleotide sequence of SEQ ID NO: 10.

In an embodiment, the CBDAS-truncated-2 gene comprises a nucleotide sequence corresponding to SEQ ID NO: 11, or a nucleotide sequence which is at least 90% identical to the nucleotide sequence of SEQ ID NO: 11.

In an embodiment, the CBDAS-truncated-3 gene comprises a nucleotide sequence corresponding to SEQ ID NO: 13, or a nucleotide sequence which is at least 90% identical to the nucleotide sequence of SEQ ID NO: 12.

In an embodiment, the CBDAS-truncated-4 gene comprises a nucleotide sequence corresponding to SEQ ID NO: 13, or a nucleotide sequence which is at least 90% identical to the nucleotide sequence of SEQ ID NO: 13.

“Cannabichromenic acid synthase” or “CBCAS” is a homodimeric oxygen dependent flavoprotein that converts CBGA to CBCA.

“Cannabichromenic acid” or “CBCA” is also a derivative of CBGA, which is converted to CBCA by CBCAS. Its neutral form, “cannabichromene” or “CBC” does not interact with the CB1 and CB2 receptors, but inhibits endocannabinoid inactivation and activates TRPA1. CBC has been associated with therapeutic or medicinal effects of cannabis and has been suggested for use as an anti-inflammatory.

In an embodiment, the at least one CBCAS gene is selected from CBCAS-1, CBCAS-2 and CBCAS-truncated.

In an embodiment, the CBCAS-1 gene comprises a nucleotide sequence corresponding to SEQ ID NO: 2, or a nucleotide sequence which is at least 90% identical to the nucleotide sequence of SEQ ID NO: 2.

In an embodiment, the CBCAS-2 gene comprises a nucleotide sequence corresponding to SEQ ID NO: 3, or a nucleotide sequence which is at least 90% identical to the nucleotide sequence of SEQ ID NO: 3.

In an embodiment, the CBCAS-truncated gene comprises a nucleotide sequence corresponding to SEQ ID NO: 4, or a nucleotide sequence which is at least 90% identical to the nucleotide sequence of SEQ ID NO: 4.

The term “gene” as used herein is to be taken in the broadest context to include the deoxyribonucleotide sequences comprising the protein coding region of a structural gene and sequences located adjacent to the coding region on both the 5′ and 3′ ends for a distance of at least about 2 kilobases (kb) on either end. The sequences which are located 5′ of the coding region and which are present on the mRNA are referred to as “5′ non-translated sequences”. The sequences which are located 3′ or downstream of the coding region and which are present on the mRNA are referred to as 3′ non-translated sequences. The term “gene” encompasses by cDNA are genomic forms of a gene. A genomic form or clone of a gene contains the coding region which may be interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide. The term “gene” includes a synthetic or fusion molecules encoding all or part of the proteins of the invention described herein and a complementary nucleotide sequence to any one of the above. The term “gene” as used herein also includes homeoforms.

An allele is a variant of a gene at a single genetic locus. Diploid cannabis such as C. sativa has two sets of chromosomes with a genome organization of XX (female) and XY (male). Each chromosome has one copy of each gene (one allele). If both alleles of a chromosome pair are the same, the organism is homozygous with respect to that gene, if the alleles are different, the organism is heterozygous with respect to that gene. The interaction between alleles at a locus is generally described as dominant or recessive.

The term “genetic modification” as used herein refers to any modification to one or more, or all of a region of a THCAS gene, at least one CBDAS gene and at least one CBCAS gene that leads to altered expression of one or more, or all of the cannabinoid biosynthesis genes.

In an embodiment, the genetic modification is targeted to the THCAS gene. In an embodiment, the genetic modification is targeted to at least one CBDAS gene. In an embodiment, the genetic modification is targeted to at least one CBCAS gene. In another embodiment, the genetic modification is targeted to all of the THCAS gene, the at least one CBDAS gene and the at least one CBCAS gene.

In an embodiment, the genetic modification is targeted to:
a. a region of at least one CBDAS gene comprising a nucleotide sequence corresponding to nucleotides 17 to 458 of SEQ ID NOs: 18 or 19, nucleotides 53 to 494 of SEQ ID NO: 20, nucleotides 191 to 632 of SEQ ID NOs: 21 or 22, nucleotides 155 to 595 of SEQ ID NO: 24, nucleotides 1 to 149 of SEQ ID NO: 25, or nucleotides 77 to 518 of SEQ ID NO: 26;
b. a region of at least one CBCAS gene comprising a nucleotide sequence corresponding to nucleotides 102 to 707 of SEQ ID NOs: 15, 16 or 17; and
c. a region of a THCAS gene comprising a nucleotide sequence corresponding to nucleotides 105 to 707 of SEQ ID NO: 14.

In an embodiment, the genetic modification is targeted to:
a. a region of at least one CBDAS gene comprising a nucleotide sequence corresponding to nucleotides 98 to 344 of SEQ ID NOs: 18 or 19, nucleotides 134 to 380 of SEQ ID NO: 20, nucleotides 272 to 518 of SEQ ID NOs: 21 or 22, nucleotides 236 to 482 of SEQ ID NO: 24, nucleotides 1 to 35 of SEQ ID NO: 25, or nucleotides 158 to 404 of SEQ ID NO: 26;
b. a region of at least one CBCAS gene comprising a nucleotide sequence corresponding to nucleotides 272 to 518 of SEQ ID NO: 15, 16 or 17; and
c. a region of a THCAS gene comprising a nucleotide sequence corresponding to nucleotides 272 to 518 of SEQ ID NO: 14.

In an embodiment, the genetic modification is targeted to a region of at least one CBDAS gene of (a).

In an embodiment, the genetic modification is targeted to a region of at least one CBCAS gene of (b).

In an embodiment, the genetic modification is targeted to a region of a THCAS gene of (c).

In another embodiment, the genetic modification is targeted to a nucleotide sequence that is shared between the regions of (a)-(c).

The term “altered” as used herein refers to a change in gene expression that results from the genetic modification.

In an embodiment, altered expression is increased expression of one or more or all of the cannabinoid biosynthesis genes. As described elsewhere herein, upregulation of one or more of the cannabinoid biosynthesis genes may be due to non-specific upregulation (Zirpel et al., 2018, Journal of Biotechnology, 284: 17-26; Fulvio et al., 2021, Plants, 10(9): 1857) or through transcription-translation interference causing upregulation, which has been demonstrated to occur using microRNAs and in mammalian siRNA systems. Variation in the level of gene silencing observed using RNAi in plants is also recognized in the art (e.g., McGinnis, 2010, Briefings in Functional Genomics, 9(2): 111-117), such that the skilled person would understand that upregulation of one or more of the cannabinoid biosynthesis genes is an alteration in gene expression mediated by the genetic modification.

The term “increased expression” as used herein means a level of expression that is higher than observed in cannabis cells in the absence of the genetic modification. In some embodiments, the level of expression of one or more cannabinoid biosynthesis genes may be increased by at least about 25% (e.g., at least about 25%, at least about 30%, at least about 35%, at least about 40%, at least about 45% at least about 50%, at least about 55%, at least about 60%, at least about 65%, at least about 70%, at least about 75%, at least about 80%, at least about 85%, at least about 90%, at least about 95%, at least about 100%, at least about 150%, at least about 200%, at least about 250%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, at least about 1000%, at least about 1100%, at least about 1200%, at least about 1300%, at least about 1400%, at least about 1500%, at least about 1600%, at least about 1700%, at least about 1800%, at least about 1900%, or at least about 2000%).

In an embodiment, altered expression is reduced expression of one or more or all of the cannabinoid biosynthesis genes.

The term “reduced expression” as used herein means a level of expression that is lower than observed in cannabis plants in the absence of the genetic modification. It is to be understood that the term “reduced” as used herein, does not necessarily imply that the expression of one or more cannabinoid biosynthesis genes has been eliminated or is reduced to an undetectable level. In some embodiments, the level of expression of one or more cannabinoid biosynthesis genes may be reduced by at least about 25% (e.g., at least about 25%, at least about 26%, at least about 27%, at least about 28%, at least about 29% at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or effectively abolished to an undetectable level, i.e., 100%).

In an embodiment, the expression of one or more cannabinoid biosynthesis gene is reduced to an undetectable level. Persons skilled in the art will appreciate that a reduction is expression to an undetectable level is intended to encompass embodiments whereby the expression of one or more cannabinoid biosynthesis gene is effectively abolished.

In an embodiment, THCAS expression is reduced by at least about 30% relative to a cannabis cell that does not comprise the genetic modification.

Accordingly, in an embodiment, THCAS expression is reduced by at least about 30% relative to a cannabis cell that does not comprise the genetic modification, preferably about 30%, preferably about 31%, preferably about 32%, preferably about 33%, preferably about 34%, preferably about 35%, preferably about 36%, preferably about 37%, preferably about 38%, preferably about 39%, preferably about 40%, preferably about 41%, preferably about 42%, preferably about 43%, preferably about 44%, preferably about 45%, preferably about 46%, preferably about 47%, preferably about 48%, preferably about 49%, preferably about 50%, preferably about 51%, preferably about 52%, preferably about 53%, preferably about 54%, preferably about 55%, preferably about 56%, preferably about 57%, preferably about 58%, preferably about 59%, preferably about 60%, preferably about 61%, preferably about 62%, preferably about 63%, preferably about 64%, preferably about 65%, preferably about 66%, preferably about 67%, preferably about 68%, preferably about 69%, preferably about 70%, preferably about 71%, preferably about 72%, preferably about 73%, preferably about 74%, preferably about 75%, preferably about 76%, preferably about 77%, preferably about 78%, preferably about 79%, preferably about 80%, preferably about 81%, preferably about 82%, preferably about 83%, preferably about 84%, preferably about 85%, preferably about 86%, preferably about 87%, preferably about 88%, preferably about 89%, preferably about 90%, preferably about 91%, preferably about 92%, preferably about 93%, preferably about 94%, preferably about 95%, preferably about 96%, preferably about 97%, preferably about 98%, preferably about 99%, or effectively abolished to an undetectable level, i.e., 100%.

In an embodiment, THCAS expression is reduced by from about 30% to about 100% relative to a cannabis cell that does not comprise the genetic modification.

In an embodiment, CBDAS expression is reduced by at least about 50% relative to a cannabis cell that does not comprise the genetic modification.

Accordingly, in an embodiment, CBDAS expression is reduced by at least about 50% relative to a cannabis cell that does not comprise the genetic modification, preferably about 50%, preferably about 51%, preferably about 52%, preferably about 53%, preferably about 54%, preferably about 55%, preferably about 56%, preferably about 57%, preferably about 58%, preferably about 59%, preferably about 60%, preferably about 61%, preferably about 62%, preferably about 63%, preferably about 64%, preferably about 65%, preferably about 66%, preferably about 67%, preferably about 68%, preferably about 69%, preferably about 70%, preferably about 71%, preferably about 72%, preferably about 73%, preferably about 74%, preferably about 75%, preferably about 76%, preferably about 77%, preferably about 78%, preferably about 79%, preferably about 80%, preferably about 81%, preferably about 82%, preferably about 83%, preferably about 84%, preferably about 85%, preferably about 86%, preferably about 87%, preferably about 88%, preferably about 89%, preferably about 90%, preferably about 91%, preferably about 92%, preferably about 93%, preferably about 94%, preferably about 95%, preferably about 96%, preferably about 97%, preferably about 98%, preferably about 99%, or effectively abolished to an undetectable level, i.e., 100%.

In an embodiment, CBDAS expression is reduced by from about 50% to about 100% relative to a cannabis cell that does not comprise the genetic modification.

In an embodiment, CBCAS expression is reduced by at least about 25% relative to a cannabis cell that does not comprise the genetic modification.

Accordingly, in an embodiment, CBCAS expression is reduced by at least about 25% relative to a cannabis cell that does not comprise the genetic modification, preferably about 25%, preferably about 26%, preferably about 27%, preferably about 28%, preferably about 29%, preferably about 30%, preferably about 31%, preferably about 32%, preferably about 33%, preferably about 34%, preferably about 35%, preferably about 36%, preferably about 37%, preferably about 38%, preferably about 39%, preferably about 40%, preferably about 41%, preferably about 42%, preferably about 43%, preferably about 44%, preferably about 45%, preferably about 46%, preferably about 47%, preferably about 48%, preferably about 49%, preferably about 50%, preferably about 51%, preferably about 52%, preferably about 53%, preferably about 54%, preferably about 55%, preferably about 56%, preferably about 57%, preferably about 58%, preferably about 59%, preferably about 60%, preferably about 61%, preferably about 62%, preferably about 63%, preferably about 64%, preferably about 65%, preferably about 66%, preferably about 67%, preferably about 68%, preferably about 69%, preferably about 70%, preferably about 71%, preferably about 72%, preferably about 73%, preferably about 74%, preferably about 75%, preferably about 76%, preferably about 77%, preferably about 78%, preferably about 79%, preferably about 80%, preferably about 81%, preferably about 82%, preferably about 83%, preferably about 84%, preferably about 85%, preferably about 86%, preferably about 87%, preferably about 88%, preferably about 89%, preferably about 90%, preferably about 91%, preferably about 92%, preferably about 93%, preferably about 94%, preferably about 95%, preferably about 96%, preferably about 97%, preferably about 98%, preferably about 99%, or effectively abolished to an undetectable level, i.e., 100%.

In an embodiment, CBCAS expression is reduced by from about 25% to about 90% relative to a cannabis cell that does not comprise the genetic modification.

In an embodiment, the genetic modification produces the altered expression of the at least one cannabinoid biosynthesis gene via gene silencing.

The term “gene silencing” as used herein refers to the reduction of expression of a target nucleic acid in a cannabis cell, preferably an endosperm cell during seed development, which can be achieved by the introduction of a silencing RNA. In some embodiments, a gene silencing chimeric gene is introduced into a cannabis cell, which encodes a RNA molecule which reduces the expression of one or more endogenous cannabinoid biosynthesis genes (e.g., a THCAS gene, at least one CBDAS gene, at least one CBCAS gene). Such reduction may be the result of reduction of transcription, including by methylation of promoter regions via chromatin re-modelling, or post-transcriptional modification of the RNA molecules, including via RNA degradation, or both. Gene silencing should not necessarily be interpreted as an abolishing the expression of the target nucleic acid or gene. It is sufficient that the level expression of the target nucleic acid in the presence of the silencing RNA is lower than that observed in the absence thereof. Accordingly, in some embodiments, the level of expression of the targeted gene may be reduced by at least about 25% (e.g., at least about 25%, at least about 26%, at least about 27%, at least about 28%, at least about 29% at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or effectively abolished to an undetectable level, i.e., 100%).

Methods for gene silencing would be known to persons skilled in the art, illustrative examples of which include the stable introduction and transcription of a gene silencing chimeric gene, sense-suppression techniques, anti-sense techniques and RNA interference (RNAi).

Antisense techniques may be used to reduce gene expression in cannabis cells. The term “antisense RNA” as used herein means an RNA molecule that is complementary to at least a portion of a specific mRNA molecule and capable of reducing expression of the gene encoding the mRNA, preferably a cannabinoid biosynthesis gene. Such reduction typically occurs in a sequence-dependent manner and is thought to occur by interfering with a post-transcriptional event such as mRNA transport from nucleus to cytoplasm, mRNA stability or inhibition of translation. The use of antisense methods is well known in the art, see, e.g., Hartmann and Endres, 1999, Manual of Antisense Methodology, Kluwer.

As used herein, the phrase “artificially-introduced dsRNA molecule” refers to the introduction of double-stranded RNA (dsRNA) molecule, which preferably is synthesized in the cannabis cell by transcription from a chimeric gene encoding such dsRNA molecule. RNA interference (RNAi) is particularly useful for specifically reducing the expression of a gene or inhibiting the production of a particular protein. This technology relies on the presence of dsRNA molecules that contain a sequence that is essentially identical to the mRNA of the gene of interest or part thereof, and its complement, thereby forming a dsRNA. Conveniently, the dsRNA can be produced from a single promoter in the host cell, where the sense and anti-sense sequences are transcribed to produce a hairpin RNA in which the sense and anti-sense sequences hybridize to form the dsRNA region with a related or unrelated sequence forming a loop structure, so the hairpin RNA comprises a stem-loop structure. The design and production of suitable dsRNA molecules for the present invention is well within the capacity of a person skilled in the art, as described by, e.g., Waterhouse et al. (1998, Proceedings of the National Academy of Science U.S.A., 95: 13959-13964; Smith et al. (2000, Nature, 407: 319-320); WO 1999/32619; WO 1999/53050; WO 1999/49029; and WO 2001/34815.

The DNA encoding the dsRNA typically comprises both sense and antisense sequences arranged as an inverted repeat. In a preferred embodiment, the sense and antisense sequences are separated by a spacer region which may (or may not) comprise an intron which, when transcribed into RNA, is spliced out. This arrangement has been shown to result in a higher efficiency of gene silencing (Smith et al., 2000, supra). The double-stranded region may comprise one or two RNA molecules, transcribed from either one DNA region or two. The dsRNA may be classified as long hpRNA, having long, sense and antisense regions which can be largely complementary, but need not be entirely complementary (typically larger than about 200 bp, e.g., between 200 and 1000 bp). A hpRNA can also be rather small with the double-stranded portion ranging in size from about 30 to about 42 bp, but not much longer than 94 bp, see, e.g., WO 2004/073390. The presence of the double stranded RNA region is thought to trigger a response from an endogenous plant system that destroys both the double stranded RNA and also the homologous RNA transcript from the target plant gene(s), efficiently reducing or eliminating the activity of the target gene.

The length of the sense and antisense sequences that hybridize should each be at least 19 contiguous nucleotides (e.g., 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 contiguous nucleotides, and so on), preferably at least 21 contiguous nucleotides, 30 contiguous nucleotides, 50 contiguous nucleotides, and more preferably at least 100, 200, 500 or 1000 contiguous nucleotides. The full-length sequence corresponding to the entire gene transcript may be used. The lengths are most preferably 100-2000 nucleotides. The degree of identity of the sense and antisense sequences to the targeted transcript should be at least 85% (e.g., at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or effectively identical, i.e., 100%), preferably at least 90% and more preferably 95-100%. The longer the sequence, the less stringent the requirement for the overall sequence identity. The RNA molecule may of course comprise unrelated sequences which may function to stabilize the molecule. The promoter used to express the dsRNA-forming construct may be any type of promoter that is expressed in the cells which express the target gene, preferably a promoter which is preferentially expressed in the endosperm of the developing cannabis seed relative to non-seed tissues of the cannabis plant.

As used herein, “silencing RNAs” are RNA molecules that have 21 to 24 contiguous nucleotides that are complementary to a region of the mRNA transcribed from the target gene, preferably one or more cannabinoid biosynthesis genes. The sequence of the 21 to 24 nucleotides is preferably fully complementary to a sequence of 21 to 24 contiguous nucleotides of the mRNA i.e., identical to the complement of the 21 to 24 nucleotides of the region of the mRNA. However, miRNA sequences which have up to five mismatches in region of the mRNA may also be used (Palatnik et al., 2003, Nature, 425: 257-263), and base-pairing may involve one or two G-U base-pairs. When not all of the 21 to 24 nucleotides of the silencing RNA are able to base-pair with the mRNA, it is preferred that there are only one or two mismatches between the 21 to 24 nucleotides of the silencing RNA and the region of the mRNA. With respect to the miRNAs, it is preferred that any mismatches, up to the maximum of five, are found towards the 3′ end of the miRNA. In a preferred embodiment, there are not more than one or two mismatches between the sequences of the silencing RNA and its target mRNA.

Silencing RNAs derived from longer RNA molecules, also referred to herein as “precursor RNAs”, are the initial products produced by transcription from the chimeric DNAs in the cannabis cells and have partially double-stranded character formed by intra-molecular base-pairing between complementary regions. The precursor RNAs are processed by a specialized class of RNAses, commonly called “Dicer(s)”, into the silencing RNAs, typically of 21 to 24 nucleotides long. “Silencing RNAs” as used herein include short interfering RNAs (siRNAs) and microRNAs (miRNAs), which differ in their biosynthesis. siRNAs derive from fully or partially double-stranded RNAs having at least 21 contiguous base-pairs, including possible G-U base-pairs, without mismatches or non-base-paired nucleotides bulging out from the double-stranded region. These double-stranded RNAs are formed from either a single, self-complementary transcript which forms by folding back on itself and forming a stem-loop structure, referred to herein as a “hairpin RNA”, or from two separate RNAs which are at least partly complementary and that hybridize to form a double-stranded RNA region. miRNAs are produced by processing of longer, single-stranded transcripts that include complementary regions that are not fully complementary and so form an imperfectly base-paired structure, so having mismatched or non-base-paired nucleotides within the partly double-stranded structure. The base-paired structure may also include G-U base-pairs. Processing of the precursor RNAs to form miRNAs leads to the preferential accumulation of one or more distinct, small RNAs each having a specific sequence, the miRNA(s). They are derived from one strand of the precursor RNA, typically the “antisense” strand of the precursor RNA, whereas processing of the long complementary precursor RNA to form siRNAs produces a population of siRNAs which are not uniform in sequence but correspond to many portions and from both strands of the precursor.

miRNA precursor RNAs, also termed herein as “artificial miRNA precursors”, are typically derived from naturally occurring miRNA precursors by altering the nucleotide sequence of the miRNA portion of the naturally-occurring precursor so that it is complementary, preferably fully complementary, to the 21 to 24 nucleotide region of the target mRNA, and altering the nucleotide sequence of the complementary region of the miRNA precursor that base-pairs to the miRNA sequence to maintain base-pairing. The remainder of the miRNA precursor RNA may be unaltered and so have the same sequence as the naturally occurring miRNA precursor, or it may also be altered in sequence by nucleotide substitutions, nucleotide insertions, or preferably deletions, or any combination thereof. The remainder of the miRNA precursor RNA is thought to be involved in recognition of the structure by the Dicer enzyme called Dicer-like 1 (DCL1), and therefore it is preferred that few if any changes are made to the remainder of the structure. For example, base-paired nucleotides may be substituted for other base-paired nucleotides without major change to the overall structure. The use of artificial miRNAs have been demonstrated in plants, see, e.g., Alvarez et al. (2006, Plant Cell, 18: 1134-1151), Parizotto et al. (2004, Genes and Development, 18: 2237-2242), and Schwab et al. (2006, Plant Cell, 18: 1121-1133).

In an embodiment, the gene silencing is mediated by small interfering RNA (siRNA).

In an embodiment, the siRNA targets THCAS and is selected from SEQ ID NOs: 27-119, or a nucleotide sequence which is at least 90% identical to the nucleotide sequence of SEQ ID NOs: 27-119.

In an embodiment, the siRNA targets a CBDAS gene and is selected from SEQ ID NOs: 120-189 and 285-322, or a nucleotide sequence which is at least 90% identical to the nucleotide sequence of SEQ ID NOs: 120-189 and 285-322.

In an embodiment, the siRNA targets a CBCAS gene and is selected from SEQ ID NOs: 190-284, or a nucleotide sequence which is at least 90% identical to the nucleotide sequence of SEQ ID NOs: 190-284.

As described elsewhere herein, the siRNA molecules of the present disclosure may tolerate a level of mismatches, depending on the length of the siRNA (e.g. up to five mismatches). It follows, therefore, that the nucleotide sequence of the siRNA described herein may also encompass a reasonable level of variation, i.e., at least 90% identical. Accordingly, the siRNA may be at least 90%, at least 91%, at least 92%, at least 93%, at least 94%, at least 95%, at least 96%, at least 97%, at least 98%, at least 99%, or 100% identical to any one of the nucleotide sequences of SEQ ID NOs: 27-322.

Methods for the determination of nucleic acid sequence identity would be known to persons skilled in the art, illustrative examples of which include computer programs that employ algorithms, such as BLAST (Atschul et al., 1990, Journal of Molecular Biology, 215(3): 403-410).

The level of cannabinoid biosynthesis gene expression can be measured using any methods known in the art for the detection and/or quantification of gene expression. Such methods would be known to persons skilled in the art, illustrative examples of which include real-time quantitative PCT (RT-qPCR), as described in Example 1.

Alternatively, or additionally, the activity of one or more cannabinoid biosynthesis proteins may be measured using any methods known in the art for the detection and/or quantification of enzyme activity, e.g., the THCAS assay described by Sirikantaramas et al. (2004, The Journal of Biological Chemistry, 279(38): 39767-39774), and the CBDAS assay described by Taura et al. (2007, FEBS Letters, 581(16): 2929-2934).

In an embodiment, the level or activity, or both of one or more cannabinoid biosynthesis proteins is reduced by reduced by at least about 40% relative to a wild-type cannabis plant. Accordingly, the level or activity or both of one or more cannabinoid biosynthesis proteins may be reduced by at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or effectively abolished to an undetectable level, i.e., 100%.

The term “control level” as used herein refers to a level (i.e., expression level) or activity that may be used to compare the characteristics of the cannabis plant or cell described herein. In certain embodiments, the control level will the level or activity in a wild-type cannabis plant or cell. The term “wild-type” as used herein refers to a cell, tissue, plant or seed that has not been modified. Wild-type cells, tissue, or plants that are known in the art may be used as controls to compare the levels of expression of endogenous or exogenous nucleic acid molecules or polypeptides, or the extent and nature of trait modification in cells, tissue, or plants modified as described herein. As used herein, the term “wild-type cannabis cell” means a cannabis cell that has not been modified, e.g., non-transformed, non-transgenic.

The skilled person will appreciate that where a comparison is made between the plants or the cell of the present disclosure and those which are wild-type, the comparison is performed with plants grown under essentially identical growing conditions, growth time, temperature, water and nutrient supply, etc., and for cells obtained from such plants.

In another embodiment, the control level is a known or predetermined level or activity that may be used to distinguish the cannabis plants or cells of the present disclosure from other cannabis plants or cells, e.g., the control level may be a pre-determined level of synthase activity that is typical of cannabis plants or cells.

In an embodiment, the cell is a regenerable cannabis cell, which is capable of growing into a plant. Regenerable cannabis cells include cells of mature embryos, meristematic tissue such as the mesophyll cells of the leaf base, or preferably from the scutella of immature embryos, obtained 12-20 days post-anthesis, or callus derived from any of these.

In an embodiment, the regenerable cell is a cell derived from an explant selected from a leaf explant, cotyledon explant and hypocotyl explant.

Accordingly, in an aspect disclosed herein there is provided a cannabis plant comprising the cell described herein, and extracts derived therefrom.

The term “plant” as used herein refers to a whole plant, parts thereof obtained from or derived from, such as, e.g., leaves, stems, roots, flowers, single cells (e.g., pollen), seeds, plant cells and the like.

The term “extract”, as used herein, is to be understood as including a whole cannabis extract, such as resin, hash and keif, as well as substantially purified compounds isolated from the harvested plant material, such as cannabinoids, terpenes and/or flavonoids.

As used herein, “substantially purified” refers to a compound or molecule that has been isolated from other components with which it is typically associated in its native state (i.e., within the plant material). Preferably, the substantially purified molecule is at least 60% free, more preferably at least 75% free, and more preferably at least 90% free from other components with which it is naturally associated. By “isolated” is meant material that is substantially or essentially free from components that normally accompany it in its native state.

### Methods for Producing a Cannabis Plant

In a another aspect of the present disclosure, there is provided a method of producing a cannabis plant with altered level or activity, or both, of a cannabinoid biosynthesis pathway, the method comprising:
a. introducing a targeted genetic modification to a cannabis cell leading to altered expression of one or more cannabinoid biosynthesis genes, wherein the genetic modification is targeted to one or more or all of
   i. a region of at least one CBDAS gene comprising a nucleotide sequence corresponding to any one of SEQ ID NOs: 18-26, or a or a nucleotide sequence which is at least 90% identical to the nucleotide sequence of SEQ ID NOs: 18-26;
   ii. a region of at least one CBCAS gene comprising a nucleotide sequence corresponding to any one of SEQ ID NOs: 15-17, or a or a nucleotide sequence which is at least 90% identical to the nucleotide sequence of SEQ ID NOs: 15-17; and
   iii. a region of a THCAS gene comprising a nucleotide sequence corresponding to SEQ ID NO: 14, or a or a nucleotide sequence which is at least 90% identical to the nucleotide sequence of SEQ ID NO: 14; and
b. regenerating a cannabis plant from the cell of step (a), wherein the plant comprises the genetic modification and has altered level or activity, or both, of the cannabinoid biosynthesis pathway relative to a cannabis plant that does not comprise the genetic modification.

In an embodiment, the genetic modification produces the altered level or activity, or both, of the cannabinoid biosynthesis genes via gene silencing.

In an embodiment, the cannabis plant is non-transgenic, i.e., does not contain an exogenous gene construct (i.e., transgene), which is preferred in some markets.

In another embodiment, the cannabis plant is a transgenic cannabis plant.

The term “transgenic cannabis plant” as used herein refers to cannabis plants and their progeny, which have been genetically modified using recombinant techniques. This would generally be to modulate the production of at least one polypeptide associated with a cannabinoid biosynthesis pathway as described herein in the desired plant or plant organ. Transgenic plant parts include all parts and cells of said plants which comprise the transgene such as, e.g., seeds, cultured tissues, callus and protoplasts. Transgenic plants contain genetic material that they did not contain prior to the transformation. The genetic material is typically stably integrated into the genome of the plant. The introduced genetic material may comprise sequences that naturally occur in the same species but in a rearranged order or in a different arrangement of elements, e.g., an antisense sequence or a sequence encoding a double-stranded RNA. Such plants are included herein in “transgenic plants”. In an embodiment, the transgenic plants are homozygous for each gene that has been introduced (e.g., transgene) so that their progeny do not segregate for the desired phenotype.

Methods for the transformation of cannabis plants to introduce an exogenous nucleic acid molecule would be known to persons skilled in the art, illustrative examples of which include acceleration of genetic material coated onto micro particles directly into cells, transformation by Agrobacterium-mediated technology, and electroporation technology. In an embodiment, transgenic cannabis plants are produced by Agrobacterium tumefaciens-mediated transformation procedures. Vectors comprising the desired nucleic acid construct may be introduced into regenerable cannabis cells of tissue cultured plants or explants, or suitable plant cells, e.g., protoplasts. In an embodiment, the cells are derived from a leaf explant, cotyledon explant and hypocotyl explant.

In an embodiment, the genetic modification is introduced by an Agrobacterium-mediated transformation method.

In another embodiment, the Agrobacterium-mediated transformation method is vacuum infiltration.

In an embodiment, the genetic modification is targeted to the THCAS gene. In an embodiment, the genetic modification is targeted to at least one CBDAS gene. In an embodiment, the genetic modification is targeted to at least one CBCAS gene. In another embodiment, the genetic modification is targeted to all of the THCAS gene, the at least one CBDAS gene and the at least one CBCAS gene.

In an embodiment, the level of the THCAS expression is reduced by at least about 30% relative to a cannabis plant that does not comprise the genetic modification.

In an embodiment, the level of THCAS expression is reduced by from about 30% to about 100% relative to a cannabis plant that does not comprise the genetic modification.

In an embodiment, the level of CBDAS gene expression is reduced by at least about 50% relative to a cannabis plant that does not comprise the genetic modification.

In an embodiment, the level of CBDAS expression is reduced by from about 50% to about 100% relative to a cannabis plant that does not comprise the genetic modification.

In an embodiment, the level of CBCAS expression is reduced by at least about 25% relative to a cannabis plant that does not comprise the genetic modification.

In an embodiment, the level of CBCAS expression is reduced by from about 25% to about 90% relative to a cannabis cell that does not comprise the genetic modification.

In an embodiment, the method further comprises crossing the plant with the genetic modification with a second plant to produce one or more progeny plants.

As used herein, the term “progeny” includes all offspring from a cannabis plant, both the immediate and subsequent generations, and both plants and seed. Progeny include the seeds and plants obtained after self-fertilization (“selfing”) and the plants resulting from a cross between two parental plants, such as the F1 offspring (first generation), F2, F3, F4, etc., being the offspring from the second etc., generations after selfing of the F1 plants.

In another embodiment, the cannabis plant is male and female fertile.

The cannabis plants described herein may be crossed with plants containing a more desirable genetic background. The desired genetic background may include a suitable combination of genes providing commercial yield or other characteristic, such as agronomic performance, biotic or abiotic stress resistance. The genetic background may also include other modification genes, e.g., genes from other cannabis lines. In some embodiments, the genetic background may comprise one or more transgenes such as, e.g., a gene that confers fungal resistance. In another embodiment, the genetic background may comprise non-transgenic mutations that confer tolerance to an herbicide.

In an embodiment, the method further comprising harvesting plant material from the plant.

As used herein, the terms “plant material” or “cannabis plant material” are to be understood to mean any part of the cannabis plant, including the leaves, stems, roots, and buds, or parts thereof, as described elsewhere herein.

In an embodiment, the plant material comprises cannabis inflorescences.

In an embodiment, the plant material is derived from a female cannabis plant. In another embodiment, the plant material is derived from a mature female cannabis plant.

In another aspect disclosed herein, there is provided a transgenic cannabis plant comprising an RNAi vector comprising a nucleotide sequence encoding a siRNA, wherein the siRNA targets one or more or all of a THCAS gene, at least one CBDAS gene and at least one CBCAS gene.

In an embodiment, the transgenic cannabis plant comprising an RNAi vector comprising a nucleotide sequence encoding a siRNA, wherein the siRNA comprises a nucleotide sequence selected from SEQ ID NOs: 27-322, or a nucleotide sequence which is at least 90% identical to the nucleotide sequence of SEQ ID NOs: 27-322.

### Polynucleotides

In another aspect of the disclosure, there is provided a synthetic siRNA molecule comprising a nucleotide sequence selected from SEQ ID NOs: 27 to 323, or a nucleotide sequence which is at least 90% identical to the nucleotide sequence of SEQ ID NOs: 27 to 323.

In an embodiment, the synthetic siRNA targets THCAS and is selected from SEQ ID NOs: 27-119, or a nucleotide sequence which is at least 90% identical to the nucleotide sequence of SEQ ID NOs: 27-119.

In an embodiment, the synthetic siRNA targets a CBDAS gene and is selected from SEQ ID NOs: 120-189 and 285-322, or a nucleotide sequence which is at least 90% identical to the nucleotide sequence of SEQ ID NOs: 120-189 and 285-322.

In an embodiment, the synthetic siRNA targets a CBCAS gene and is selected from SEQ ID NOs: 190-284, or a nucleotide sequence which is at least 90% identical to the nucleotide sequence of SEQ ID NOs: 190-284.

The terms “polynucleotide”, “nucleic acid” and “nucleic acid molecule” refer to a single- or double-stranded polymer of deoxyribonucleotide, ribonucleotide bases or known analogs of natural nucleotides, or mixtures thereof, and can include molecules comprising coding and non-coding sequences of a gene, sense and anti-sense sequences and complements, exons, introns, genomic DNA, cDNA, pre-mRNA, mRNA, rRNA, siRNA, miRNA, tRNA, ribozymes, recombinant polynucleotides, isolated and purified naturally-occurring sequences, synthetic RNA and DNA sequences, nucleic acid probes, primers and fragments.

The phrase “synthetic siRNA” as used herein, refers to non-naturally occurring siRNA molecules (i.e., polynucleotides). Suitable methods for producing synthetic siRNA would be known to persons skilled in the art, illustrative examples of which include by chemical synthesis, in vitro transcription, digestion of long dsRNA by an RNase III family enzyme (e.g., Dicer, RNase III), and recombinant expression from an RNAi vector.

In an embodiment, the synthetic siRNA molecules described herein are substantially purified polynucleotides.

As used herein, “substantially purified polynucleotide” refers to a polynucleotide (i.e., a synthetic siRNA) that has been separated from the lipids, nucleic acids, other peptides and other molecules with which it is naturally associated in its native state. Preferably, the substantially purified polynucleotide is at least 60% free, more preferably at least 75% free, and more preferably at least 90% free from other components with which it is naturally associated. By “recombinant polynucleotide” it is meant a polypeptide made using recombinant techniques, e.g., through the expression of a recombinant polynucleotide in a cell, preferably a cannabis cell.

In accordance with certain embodiments of the present disclosure, the synthetic siRNA molecule is in a cell, preferably in a cannabis cell, or in plant material or extracts derived therefrom.

Transformation of a nucleic acid molecule into a cell can be accomplished by any method by which a nucleic acid molecule can be inserted into the cell. Transformation techniques include, but are not limited to, transfection, electroporation, microinjections, lipofection, adsorption, and protoplast fusion.

The synthetic siRNA molecule of the present disclosure may be operably linked to a promoter capable of driving expression of the nucleic acid molecule in a plant cell.

“Operably linking” a promoter or enhancer element to a synthetic siRNA molecule means placing the synthetic siRNA molecule (e.g., a nucleic acid molecule encoding a synthetic siRNA molecule) under the regulatory control of a promoter, which then controls the transcription of that polynucleotide. In the construction of heterologous promoter/structural gene combinations, it is generally preferred to position a promoter or variant thereof at a distance from the transcription start site of the nucleic acid molecule, which is approximately the same as the distance between that promoter and the gene it controls in its natural setting, i.e., the gene from which the promoter is derived. As is known in the art, some variation in this distance can be accommodated without loss of function.

In certain embodiments, a nucleic acid molecule encoding the synthetic siRNA is comprised in a vector (i.e., an RNAi vector).

Such a vector contains heterologous nucleic acid sequences, that is, nucleic acid sequences that are not naturally found adjacent to nucleic acid molecules of the present invention, which are preferably derived from a species other than the species from which the nucleic acid molecule(s) are derived. The vector can be either RNA or DNA, either prokaryotic or eukaryotic, and is typically is a virus or a plasmid.

In an embodiment, the nucleic acid molecule encoding the synthetic siRNA will be comprised in a nucleic acid construct, e.g., incorporated into the vector described herein. Nucleic acid constructs typically comprise one or more regulatory elements, such as promoters, enhancers, as well as transcription termination or polyadenylation sequences. Such elements are well known in the art. The transcription initiation region comprising the regulatory element(s) may provide for regulated or constitutive expression in the plant.

Typically, the nucleic acid construct comprises a selectable marker. Selectable markers aid in the identification and screening of plants or cells that have been transformed with an exogenous nucleic acid molecule. The selectable marker may provide antibiotic or herbicide resistance to the cannabis cells, or allow the utilization of substrates, e.g., mannose.

In an embodiment, the nucleic acid construct is for transient expression and does not comprise a selectable marker. The use of nucleic acid constructs without a selectable marker beneficially allows vectors to be smaller and easier to handle.

In an embodiment, the nucleic acid construct is stably incorporated into the genome of a plant. Accordingly, the nucleic acid construct comprises additional elements, which allow the molecule to be incorporated into the genome, or the construct is placed in an appropriate vector, which can be incorporated into a chromosome of a plant cell.

Methods for Selecting a Short Interfering RNA (siRNA)

In another aspect of the present disclosure, there is provided a method for selecting a short interfering RNA (siRNA) for altering the expression of one or more cannabinoid biosynthesis genes, the method comprising:
a. providing a set of potential siRNA which target one or more or all of a THCAS gene, at least one CBDAS gene and at least one CBCAS gene;
b. determining the binding complementarity of the siRNA to:
   i. a region of at least one CBDAS gene;
   ii. a region of at least one CBCAS gene; and
   iii. a region of a THCAS gene; and
c. selecting an siRNA that has on-target binding complementarity to regions (i)-(iii) and negligible off-target binding complementarity to a region outside of regions (i)-(iii).

As used herein, the terms “complementarity” and “complementary” refer to a nucleic acid that can form one or more hydrogen bonds with another nucleic acid sequence by either traditional Watson-Crick or other non-traditional types of interactions. In reference to the siRNA molecules disclosed herein, the binding free energy of a siRNA molecule with its target (i.e., complementary) sequence is sufficient to allow the relevant function of the siRNA to proceed, in some embodiments, to form a duplex structure under physiological conditions in a plant cell, to mediate ribonuclease activity, etc.

In an embodiment, the level of complementarity is described by reference to the percentage of residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). The terms “100% complementary”, “fully complementary”, and “perfectly complementary” indicate that all of the contiguous residues of a nucleic acid sequence can hydrogen bond with the same number of contiguous residues in a second nucleic acid sequence.

The terms “target RNA” or “target sequence” as used herein refer to an RNA to be modulated (e.g., inhibited by an RNA silencing mechanism). In certain embodiments, the target sequence is incorporated into the RNA-induced silencing complex (RISC) and cleaved in a sequence-specific manner as directed by a siRNA that binds to a portion of the target. The cleavage products are then further degraded.

The term “target gene” as used herein refers to a gene that encodes a target RNA (e.g., a THCAS gene, a CBDAS gene, a CBCAS gene). As used herein, a “target gene” need not be a full-length gene or encode a full-length RNA, but may instead be or encode a portion thereof, e.g., a portion of an open reading frame, 5′ or 3′ untranslated region, exon(s), intron(s), flanking region, etc. The target gene, target sequence, target RNA, and the like, are said to be “targeted” by a siRNA that mediates inhibition thereof, i.e., mediates cleavage of the transcript.

Accordingly, the term “on-target” as used herein refers to exact sequence homology matches (i.e., fully complementary) within a cannabinoid biosynthesis gene selected from a THCAS gene, at least one CBDAS gene and at least one CBCAS gene.

By contrast, the term “off-target” as used herein refers to exact sequence homology matches in a different cannabinoid biosynthesis gene. For example, in the instance of pRNAi-GG-CBDAS-UNIVERSAL, an off-target is defined as an exact match that does not reside within the CBDAS-truncated-4 gene, e.g., THCAS, CBDAS-1, CBDAS-2, CBDAS-like-1, CBDAS-like-2, CBDAS-like-3, CBDAS-truncated-1, CBDAS-truncated-2, CBDAS-truncated-3, CBCAS-1, CBCAS-2, or CBCAS-truncated.

By limiting the off-target binding complementarity to cannabinoid biosynthesis genes, the present inventors' have demonstrated that it is possible to downregulate the expression of multiple cannabinoid biosynthesis genes simultaneously. Accordingly, in some embodiments, the siRNA selected in accordance with the methods disclosed herein may be variously described as “universal siRNA” or “universal cannabinoid biosynthesis siRNA”.

In an embodiment, the selected siRNA has on-target binding complementarity in a region selected from:
a. a region of at least one CBDAS gene comprising a nucleotide sequence corresponding to any one of SEQ ID NOs: 18-26, or a or a nucleotide sequence which is at least 90% identical to the nucleotide sequence of SEQ ID NOs: 18-26;
b. a region of at least one CBCAS gene comprising a nucleotide sequence corresponding to any one of SEQ ID NOs: 15-17, or a or a nucleotide sequence which is at least 90% identical to the nucleotide sequence of SEQ ID NOs: 15-17; and
c. a region of a THCAS gene comprising a nucleotide sequence corresponding to SEQ ID NO: 14, or a or a nucleotide sequence which is at least 90% identical to the nucleotide sequence of SEQ ID NO: 14.

In an embodiment, the selected siRNA has on-target binding complementarity in a region selected from:
a. a region of at least one CBDAS gene comprising a nucleotide sequence corresponding to nucleotides 17 to 458 of SEQ ID NOs: 18 or 19, nucleotides 53 to 494 of SEQ ID NO: 20, nucleotides 191 to 632 of SEQ ID NOs: 21 or 22, nucleotides 155 to 595 of SEQ ID NO: 24, nucleotides 1 to 149 of SEQ ID NO: 25, or nucleotides 77 to 518 of SEQ ID NO: 26;
b. a region of at least one CBCAS gene comprising a nucleotide sequence corresponding to nucleotides 102 to 707 of SEQ ID NOs: 15, 16 or 17; and
c. a region of a THCAS gene comprising a nucleotide sequence corresponding to nucleotides 105 to 707 of SEQ ID NO: 14.

In an embodiment, the selected siRNA has on-target binding complementarity in a region selected from:
a. a region of at least one CBDAS gene comprising a nucleotide sequence corresponding to nucleotides 98 to 344 of SEQ ID NOs: 18 or 19, nucleotides 134 to 380 of SEQ ID NO: 20, nucleotides 272 to 518 of SEQ ID NOs: 21 or 22, nucleotides 236 to 482 of SEQ ID NO: 24, nucleotides 1 to 35 of SEQ ID NO: 25, or nucleotides 158 to 404 of SEQ ID NO: 26;
b. a region of at least one CBCAS gene comprising a nucleotide sequence corresponding to nucleotides 272 to 518 of SEQ ID NO: 15, 16 or 17; and
c. a region of a THCAS gene comprising a nucleotide sequence corresponding to nucleotides 272 to 518 of SEQ ID NO: 14.

In an embodiment, the selected siRNA has on-target binding complementarity in a region of a THCAS gene, and wherein the selected siRNA has off-target binding complementarity to a non-target sequence in a region of one or both of at least one CBDAS gene and at least one CBCAS gene.

In an embodiment, the selected siRNA has on-target binding complementarity in a region of at least one CBDAS gene, and wherein the selected siRNA has off-target binding complementarity to a non-target sequence in a region of one or both of a THCAS gene and at least one CBCAS gene.

In an embodiment, the selected siRNA has on-target binding complementarity in a region of at least one CBCAS gene, and wherein the selected siRNA has off-target binding complementarity to a non-target sequence in a region of one or both of a THCAS gene and at least one CBDAS gene.

In another aspect disclosed herein, there is provided a method of altering the expression of cannabinoid biosynthesis genes in a cannabis cell, the method comprising transiently transfecting a cannabis cell with an RNAi vector comprising a nucleotide sequence encoding a siRNA via agroinfiltration, wherein the siRNA targets one or more or all of a THCAS gene, at least one CBDAS gene and at least one CBCAS gene.

In another aspect disclosed herein, there is provided a method of altering the expression of cannabinoid biosynthesis genes in a cannabis cell, the method comprising stably transducing a cannabis cell with an RNAi vector comprising a nucleotide sequence encoding a siRNA, wherein the siRNA targets one or more or all of a THCAS gene, at least one CBDAS gene and at least one CBCAS gene.

In an embodiment, the siRNA has on-target binding complementarity to:
a. a region of at least one CBDAS gene;
b. a region of at least one CBCAS gene; and
c. a region of a THCAS gene, wherein the siRNA has negligible off-target binding complementarity to a region outside of regions (a)-(c).

The term “agroinfiltration” as used herein refers to a transient transformation method, which enables safe, high-level and rapid transgene expression as compared to the establishment of transgenic plants. As described in Example 1, agroinfiltration may be performed using Agrobacterium tumefaciens to deliver a vector comprising the siRNA of interest into the extracellular leaf spaces by physical or vacuum infiltration.

The term “stably transducing” as used herein refers to the transduction of cannabis cells with an RNAi vector, which enables transgene expression and the establishment of transgenic plants. As described in Example 1, stable transduction may be performed using Agrobacterium tumefaciens to deliver a vector comprising the siRNA of interest into regenerable plant cells such as hypocotyl and cotyledon cells, which can be used to regenerate a cannabis plant.

All publications mentioned in this specification are herein incorporated by reference. The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

It will be appreciated by persons skilled in the art that numerous variations and/or modifications may be made to the present disclosure without departing from the spirit or scope of the disclosure as broadly described. The present embodiments are, therefore, to be considered in all respects as illustrative and not restrictive.

The present disclosure will now be further described in greater detail by reference to the following specific examples, which should not be construed as in any way limiting the scope of the disclosure.

### EXAMPLES

### Example 1—General Materials and Methods

### Alteration of Cannabinoid Biosynthesis Via Transient RNAi Expression

### Plant Material and Growth Conditions

Leaf material from a C. sativa cultivar with a known ratio of THC to CBD (1:1.8 THC:CBD) was used for transient expression experiments. Cannabis plants were propagated in 9 L plastic pots using coco-coir and grown using hydroponics nutrients (THC, Australia) as per manufacturer's recommended nutrient strength, in a controlled greenhouse environment at 25° C. Leaf explants were chosen from newly developing shoot meristems on approximately 2-month-old donor plants grown under high pressure sodium grow lights, 500 μmol m⁻²s⁻¹, with a photoperiod of 18 h light and 8 h dark regime.

Identification of Candidate Genes, siRNA Design and Gene Amplification

Sequence data of the endogenous THCAS, CBDAS and CBCAS genes were accessed from the cannabis plant genome assembly (Braich et al., 2020, Biochemical Pharmacology, 85(9): 1306-1316). THCAS, CBDAS and CBCAS gene sequences were determined by BLAST querying the genome assembly with an e-value threshold set at <10⁻¹⁰, as shown in SEQ ID NOs: 1-13. Exons from the gene sequences were predicted using FGENESH (Solovyev et al., 2006, Genome Biology, 7(1): S10) and ExPASy (Gasteiger et al., 2003, Nucleic Acids Research, 31(13): 3784-3788), as shown in SEQ ID NOs: 14-26. Predicted gene sequences were viewed and aligned using Geneious Prime 2020.2. siRNAs from amplified gene sequences were predicted using pssRNAit (Ahmed, et al., 2020, Plant Physiology, 184(1): 65-81), using the recommended parameters, to generate a library of siRNA fragments within the chosen gene sequences. The number of off-target sites within the cannabinoid biosynthesis genes was predicted by BLASTn analysis of each siRNA sequence, recording the total number of exact sequence homology matches, with off-targeting determined as an exact sequence residing within a different biosynthesis gene set. For example, in the instance of pRNAi-GG-CBDAS-UNIVERSAL an off-target is defined as an exact match that does not reside within the CBDAS-truncated-4 gene. The antisense sequence of each siRNA correspond to SEQ ID NOs: 27-322.

Primers were designed, using Primer3 (Untergasser et al., 2012, Nucleic Acids Research, 40(15): e111), in gene regions of sequence variance and homology, with products between ˜250 to ˜600 base pairs for siRNA generation in vivo (Table 2). Each forward and reverse primer had the 5′ adapter sequences “acca ggtctc aggag” (see, e.g., nucleotides 1-15 of SEQ ID NO: 323) and “acca ggtctc atcgt” (see, e.g., nucleotides 1-15 of SEQ ID NO: 324), respectively. DNA fragments were PCR amplified from genomic DNA, using Phusion polymerase (New England Biolabs, Ipswich, MA) with PCR cycling as follows: 98° C. 30 see, 35 cycles of 98° C. 10 see, 60° C. 30 see, 72° C. 30 see, final extension 72° C. 10 min.

### Plasmid Construction, Agrobacterium Culture Conditions and Vacuum Infiltration

For expression of siRNAs, pRNAi-GG vector was used within this study. pRNAi-GG was provided by The Arabidopsis Biological Resource Center (TAIR). The construction of the vectors containing gene sequences of interest was followed according to a previously published protocol (Yan et al., 2012, PLOS One, 7(5): e38186). Briefly, 50 ng of purified PCR products were mixed with 200 ng of pRNAi-GG with 5 units of Bsal (New England Biolabs, Ipswich, MA) and 10 units of T4 Ligase (New England Biolabs, Ipswich, MA) in a total volume of 20 μl in T4 ligation buffer. Restriction-ligation was carried out at 37° C. for 2 h followed by a final digestion at 50° C. for 5 min and heat inactivation at 80° C. for 5 min. E. coli DH5a competent cells were transformed with 5 μl of the mixture and plated on LB media containing 25 mg/L kanamycin and 5 mg/L chloramphenicol.

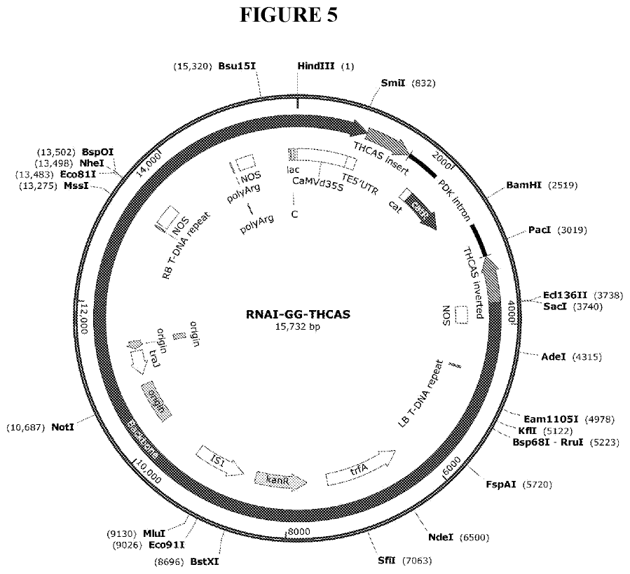

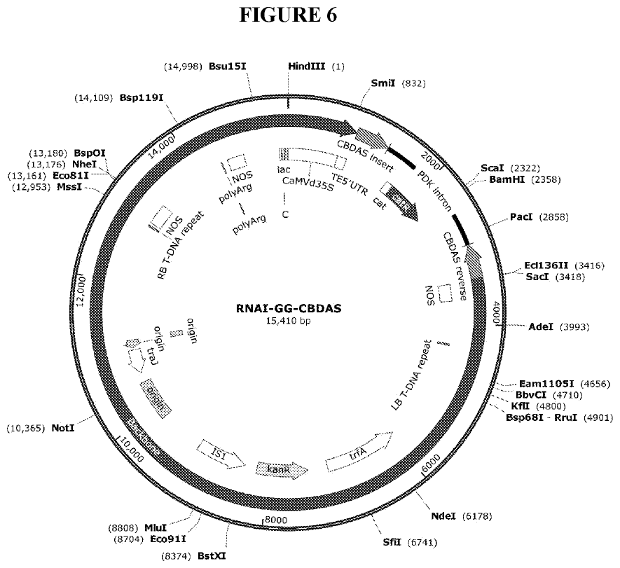

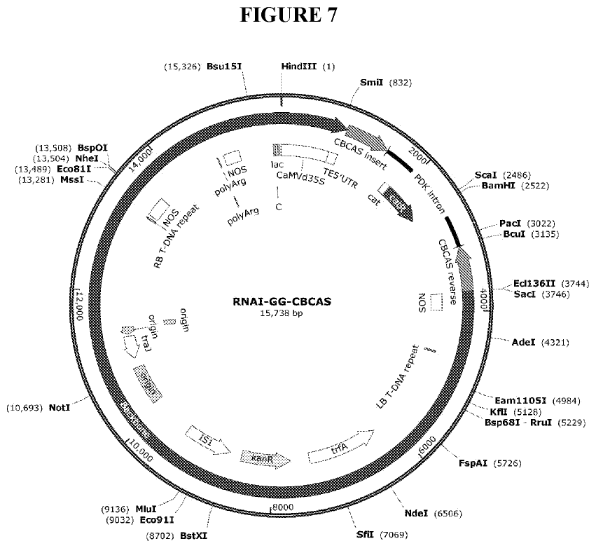

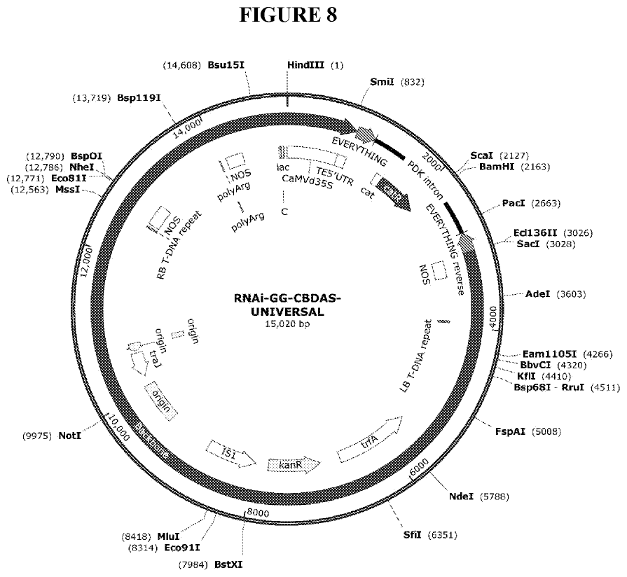

Recombinant bacterial colonies were PCR verified with primers flanking the PCR product insert (Table 3; SEQ ID NOs: 331-334) and bands were visualized using a TapeStation 2200 (Agilent, Santa Clara, CA) with colonies of expected band sizes sequence verified. Final constructs were labelled pRNAi-GG-THCAS, pRNAi-GG-CBDAS, pRNAi-GG-CBCAS and pRNAi-GG-CBDAS-UNIVERSAL (comprising the cDNA inserts corresponding to SEQ ID NOs: 335-338; FIGS. 5-8).

Recombinant Agrobacterium tumefaciens strains were generated via electroporation in accordance with the method of Lin (1995, Electroporation Protocols for Microbiology, 171-178). Agrobacterium culture conditions and vacuum infiltration protocols were as described by Deguchi et al. (2020, Scientific Reports, 10(1): 1-11) with slight modifications. Briefly, for the expression of pRNAi-GG constructs, A. tumefaciens strain GV3101 was used for transient expression experiments. Recombinant A. tumefaciens were inoculated and grown in YM media (0.5 g/L K₂HPO₄, 0.2 g/L MgSO₄₋₇H₂O, 0.1 g/L NaCl, 10 g/L mannitol, 0.4 g/L yeast extract, pH 7) overnight at 220 rpm at 30° C. The culture was centrifuged at 4,000 g for 10 min and resuspended to an OD₆₀₀=0.5 in infiltration media (10 mM MES, 1×MS and vitamins, 2% glucose, 200 μM acetosyringone, pH 5.6) and placed on a rotary shaker (Ratek, Australia) for 2 h prior to vacuum infiltration. Immediately before infiltration, 5 mM ascorbic acid, 0.05% (v/v) Pluronic F-68 and 0.015% (v/v) Silwett L-77 (Sigma Aldrich, St. Louis, MO) was added to the A. tumefaciens culture.

Leaf discs (approx. 2 cm×2 cm) were taken from young fully expanded leaves of ca. 2-month-old, donor cannabis plants and placed in a Petri dish (100 mm×15 mm) containing A. tumefaciens suspension. The Petri dish was then placed in a desiccator for 2 min at 400 mbar with vacuum pressure gently released. Vacuum was reapplied once more allowing thorough infiltration. Leaf material was washed with sterile water and transferred onto moist (ddH₂O) filter paper in a Petri dish and placed in a controlled environment room at 24° C. with an 18 h photoperiod for 4 days.

### Quantitative Real-Time PCR (RT-qPCR) Analysis of Agroinfiltrated Leaf Discs

72 h post-vacuum agroinfiltration, leaf discs were snap frozen in liquid nitrogen and total RNA was extracted following manufacturer's instructions (RNeasy Plant Mini Kit, Qiagen, Hilden, Germany). cDNA synthesis and qPCR were carried out in one step with Luna Universal One-Step RT-qPCR Kit (New England Biolabs, Ipswich, MA) following manufacturer's instructions. Quantitative PCR parameters used were as follows: 95° C. for 60 sec, 40 cycles at 95° C. for 15 sec and 59° C. for 15 sec carried out with a CFX-96 Touch Real-Time PCR Detection System (Bio-Rad, Hercules, CA). Melting curves were measured and gene expression levels were calculated from the cycle threshold according to the 2^{-ΔΔCt} method (Livak and Schmittgen, 2001, Methods, 25(4): 402-408). Paired t-test was performed (p=0.05) to determine significance using RStudio (Version 1.1.453, RStudio Inc., Boston, MA). The UBQ5 gene was used as an internal reference (Deguchi et al., 2020, supra), with three biological replicates used for all qPCR experiments with two technical replicates. All qPCR primer sequences are listed in Table 4.

Alteration of Cannabinoid Biosynthesis Via Transformation and Regeneration with an Integrated RNAi Vector

### Plant Material and Agrobacterium Culture Conditions

Cotyledon explants from a segregating F2 inter-cross between a high THC male and high CBD female cultivar were used for transformation experiments. All seeds were initially surface sterilized by soaking in 80% (v/v) ethanol for 1 minute with gentle shaking. Seeds were then rinsed with sterile ddH₂O three times and soaked in 15% (v/v) bleach (4.25% active sodium hypochlorite) for 10 minutes with gentle agitation. Seeds were rinsed three times again in ddH₂O and approximately 50 seeds were placed in each 50 mL centrifuge tube half filled with sterile ddH₂O for 5-7 days in a controlled environment room at 24° C. in the dark for germination to enable cotyledon excision. Sterile seeds used for hypocotyl excision were placed in germination medium [1/2 MS and vitamins, 1.5% sucrose, 3.5 g/L Gelrite, pH 5.8] in Stericon-8 culture vessels for 7-14 days in a controlled environment room at 24 degrees with a 16/8 photoperiod, providing 74 μmol m⁻² s⁻¹ light intensity supplied by fluorescent lamps.

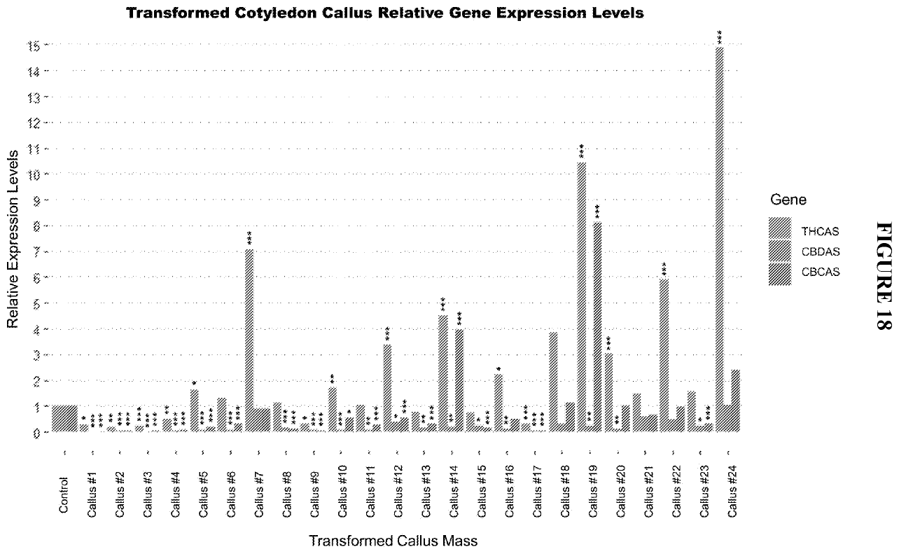

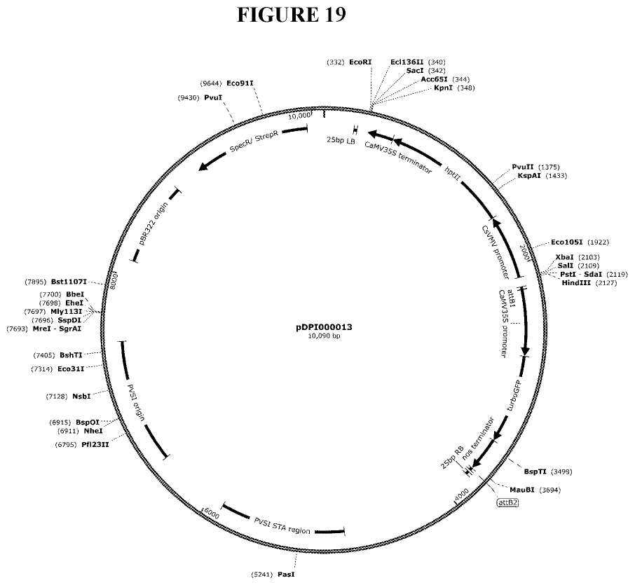

For the expression of siRNA, the pRNAi-GG-CBDAS-UNIVERSAL vector was used. Cotyledons and hypocotyls were also co-infiltrated with the binary vector pDPI-13, constructed from a pPZP200-based binary vector with CaMV35S-p turboGFP_nos-t and CsVMV-p_hph_CaMV35S-t cassettes and spectinomycin selection, to express GFP (FIG. 19). Recombinant Agrobacterium strain GV3101 was used for all transformation experiments. Agrobacterium was grown overnight in YM media [0.5 g/L K₂HPO₄, 0.2 g/L MgSO₄-7H₂O, 0.1 g/L NaCl, 10 g/L Mannitol, 0.4 g/L yeast extract, PH 7] with appropriate selection at 200 rpm at 30° C. The cultures were centrifuged at 4000 g for 10 min and resuspended to OD₆₀₀=0.5 in infiltration media [10 mM MES, 1×MS, 2% glucose, pH 5.6] and mixed prior to transformation.

### Cotyledon and Hypocotyl Excision, Agrobacterium-Mediated Transformation and Co-Cultivation

Once seeds were germinated, for cotyledon transformation, the seed coat was pried open using sterile forceps and scalpel with the cotyledons removed by cutting the two cotyledons apart and removing the radicle. The cotyledons were placed into the mixed Agrobacterium cultures for 15 minutes in the dark at room temperature (22° C.). Post infiltration, cotyledons were placed on sterile filter paper for 5 minutes to remove excess Agrobacterium culture. Cotyledons were moved onto solid co-cultivation media [1×MS and vitamins, 3% sucrose, 0.5 g/L MES hydrate, 0.7% agar, 2 mg/L 2,4-D, 200 μM acetosyringone, pH 5.6] for 3 days in the dark at 24° C. Control cotyledons were treated identically, however, a disarmed Agrobacterium was used.

Hypocotyls were excised from germinated seeds and placed in Agrobacterium culture for 40 minutes in the dark at room temperature for static infection. Post-infection, hypocotyls were placed on sterile filter paper for 5 minutes to remove excess Agrobacterium culture. Hypocotyls were then transferred to co-cultivation media [1/2 MS and vitamins, 1.5% sucrose, 200 μM acetosyringone, 3.5 g/L Gelrite, pH 5.6] for 3 days in a controlled environment room as described above. Similar to cotyledons, control hypocotyls were generated using a disarmed Agrobacterium.

### Callus Induction and Regeneration

Cotyledon callus induction was carried out on callus induction media (CIM) [1×MS and vitamins, 3% sucrose, 0.7% agar, 1 mg/L Kinetin, 0.2 mg/L NAA, pH 5.8]. Three days post co-cultivation, cotyledons were rinsed 3 times with sterile ddH₂O and washed with 200 mg/L Timentin for 2 minutes. Post-wash, cotyledons were moved to filter paper for 5 minutes to remove excess moisture before being moved onto CIM, 12 per plate, and placed in an incubator at 24° C. in the dark for 5 weeks. Phenotypic development of callus was photographed weekly and images analyzed using ImageJ (ImageJ 1.53e, National Institute of Mental Health, MD, USA) using the free-hand tool. Callus mass on selected sub-set was performed on an electronic balance after 14 days initiation and weighed every consecutive 7 days.

Cotyledon regeneration media [1×MS and vitamins, 2% maltose, 0.1 g/L myo-inositol, 10 mM MES, 0.7% agar, 120 mg/L Timentin, pH 5.8] was prepared with three increasing concentrations of Thidiazuron (TDZ; 2.5, 5 and 10 μM). Post-callus induction, calli was transferred onto regeneration media in SteriCon-13 culture vessels for 6-8 weeks.

Post co-cultivation, hypocotyls were rinsed with sterile ddH₂O three times and placed in 200 mg/L Timentin wash for 5 minutes with occasional gentle agitation. Post-Timentin wash, hypocotyls were placed on filter paper for 5 minutes and then transferred to regeneration media [1/2 MS and vitamins, 1.5% sucrose, 3.5 g/L Gelrite, 120 mg/L Timentin, pH 5.8] for 4-8 weeks in Stericon-8 culture vessels in a controlled environment room as described above. Regenerated shoots were excised and moved to rooting media [1/2 MS and vitamins, 1% sucrose, 5 μM IBA, 1% agar, 120 mg/L Timentin, pH 5.8].

### RT-qPCR Analysis of Transformed Calli and Hypocotyl

Approximately 100 mg callus mass was snap frozen in liquid nitrogen with total RNA extracted following manufacturers instruction (RNeasy Plant Mini Kit, Qiagen, Hilden, Germany) and quantified using a Nanodrop 1000 (ThermoFisher Scientific, Walthan, MA). Approximately 5 μg of RNA was treated with DNase I (New England Biolabs, Ipswich, MA) as per manufacturer's instructions and used for subsequent RT-qPCR. Due to the numerous pseudogenes within the CBDAS locus, two primer pairs were designed, one set targeting the known functional copy of CBDAS, and one set designed to target CBDAS-like homologs and mixed in equal molar concentrations to target all possible CBDAS-homologs during RT-qPCR. All qPCR primer sequences are listed in Table 4.

### GFP Expression Detection and Analysis

Cotyledons, hypocotyls, callus and leaf material were imaged under fluorescence with GFP filter set (Excitation 395-455 nm, emission 480 nm) using a Leica camera (CH-9435) with Leica Application Suite software (4.12.0). GFP fluorescence data for each callus was collected at week 5, prior to moving to regeneration media to determine gene stability. Hypocotyls were measured after 5 weeks on regeneration media. Data was collected as presence/absence in explant material.

### Example 2—Identification of Cannabinoid Genes and siRNA Prediction

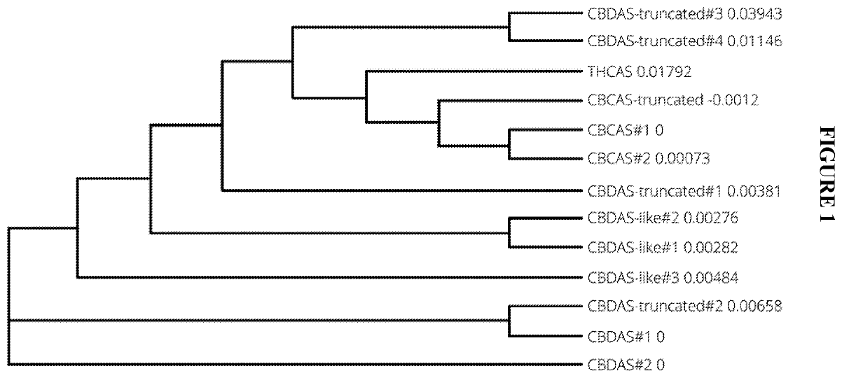

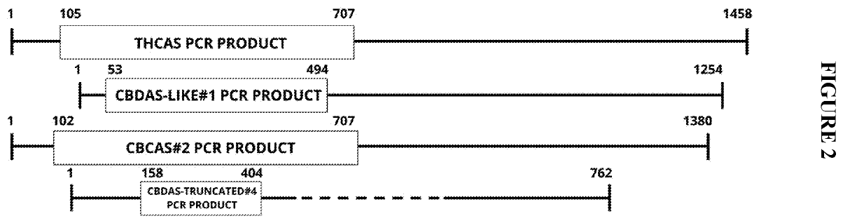

THCAS, CBDAS and CBCAS gene sequences were determined by BLAST querying the cannabis genome sequence assembly with publicly available sequences (SEQ ID NOs: 1-13). Each cannabinoid biosynthesis gene, and accompanying homologs, were analyzed for functionality using FGENESH and ExPASy, before BLASTn analysis for homology to publicly available sequences and pairwise aligned using MUSCLE to create a phylogenetic tree (FIG. 1) and a matrix with identity percentages of coding sequences (Table 6).

Within the genome of the cannabis plant used for this study, a single functional copy of THCAS exists, however CBDAS and CBCAS contain nine and three homologs/pseudogenes, respectively. Using FGENESH and ExPASy, two identical CBDAS genes were discovered (CBDAS-1 and CBDAS-2), and three homologs were identified containing several single nucleotide polymorphisms (SNPs) leading to differences in predicted protein translations (CBDAS-like-1, CBDAS-like-2, CBDAS-like-3) and four copies of CBDAS were found to be truncated when proteins were predicted (CBDAS-truncated-1, CBDAS-truncated-2, CBDAS-truncated-3, CBDAS-truncated-4). The coding sequences (CDS) of each CBDAS homologs were aligned and non-truncated homologs are shown to be >86% homologous. The high levels of sequence similarity of the CBDAS homologs (Table 6) at the DNA level, and regardless of the size of the PCR insert for siRNA generation, sequence homology is too significant to identify one best-fit homolog for vector design and thus CBDAS-like-1 was selected for pRNAi-GG-CBDAS vector construction.

Two functional copies of CBCAS were found (CBCAS-1 and CBCAS-2) having identical sequence homology, except for base pair 482, where a synonymous SNP occurs (T to C), however, this variant is not predicted to affect the translated proteins. A truncated CBCAS homolog was also identified of 969 bp was designated CBCAS-truncated. CBCAS-2 was chosen for pRNAi-GG-CBCAS vector construction.

A significantly smaller sequence (247 bp), homologous to the CBDAS-truncated-4 homolog, was chosen in a region of high homology from the sequence alignment of all cannabinoid synthesis genes CDS, however lower in homology (<90%) within the subset of CBDAS sequences, designated ‘UNIVERSAL’ to determine if a smaller gene sequence for RNAi containing lower homology could be more effective in gene silencing through off-targeting. A graphic representation for the alignment of cannabinoid biosynthesis genes, with the PCR products sizes, are shown in FIG. 2.

The gene sequences selected for RNAi were analyzed using pssRNAit to assess the degree of off targeting to the identified cannabinoid gene sequences for each specific vector. Efficient gene silencing requires the formed siRNA to contain minimal off-targeting silencing effects. The siRNA sequences generated by pssRNAit are presented in SEQ ID NOs: 27-323. From the amplified THCAS sequence, 93 siRNA were predicted with 1609 potential off-targets, CBDAS with 70 predicted siRNA and 1609 potential off-targets, CBCAS with 95 predicted siRNA and 1647 potential off-targets and UNIVERSAL with 38 predicted siRNA with 630 potential off-targets (Table 7).

To filter out irrelevant off-target sites not residing within the cannabinoid genes, each siRNA was aligned to the cannabinoid biosynthesis genes for sequence similarity to greater understand off-targeting potential within these highly homologous sequences. A total number of 369 exact targets for pRNAi-GG-THCAS exist within the cannabinoid biosynthesis genes with 93 exact matches to THCAS and 276 off-targets existing within the other gene sets (Table 7). pRNAi-GG-CBDAS contained 447 total exact targets within all biosynthesis genes, with 381 targeting a minimum of 1 CBDAS homologs and containing considerably more off-targets tallying 64 sites not residing within CBDAS homologs (Table 7). pRNAi-GG-CBCAS contained a similar number of total targets, 428, with 276 targets within CBCAS homologs and contained substantially more off-targets, with 152 exact matches across other gene sets (Table 7). Within the pRNAi-GG-CBDAS-UNIVERSAL predicted siRNA, only 69 exact targets exist within all cannabinoid biosynthesis genes. A total of 38 siRNA sites exist within the predicted CBDAS-truncated-4 gene sequence, with the remaining 31 target sites residing within CBDAS homologs (Table 7).

### Example 3—Vector Construction, Generation of Recombinant Agrobacterium and Vacuum Infiltration

To test the efficiency of silencing cannabinoid biosynthesis genes, recombinant expression vectors were made for the four target sequences (FIGS. 5-8). The vectors contained sense-antisense orientation separated by an intron and were cloned into an E. coli strain.

Eight recombinant colonies were chosen, for each treatment, for colony PCR using sequence specific primers residing within the specific sequence and residing on the vector backbone. All clones showed the expected bands confirming the correct inserts, which were subsequently sequenced to confirm the correct sequences as expected.

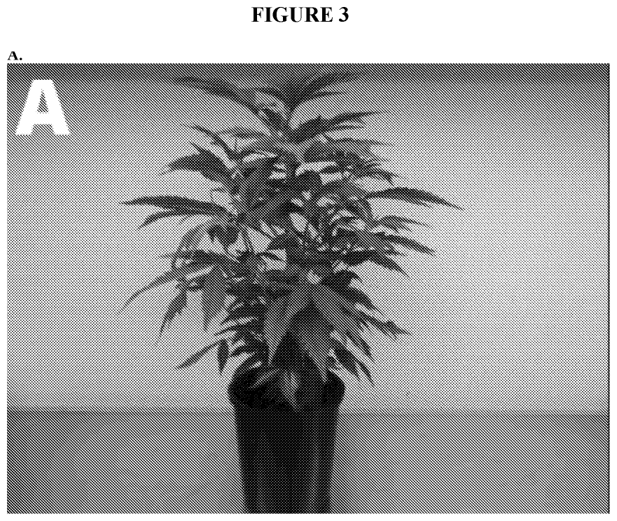

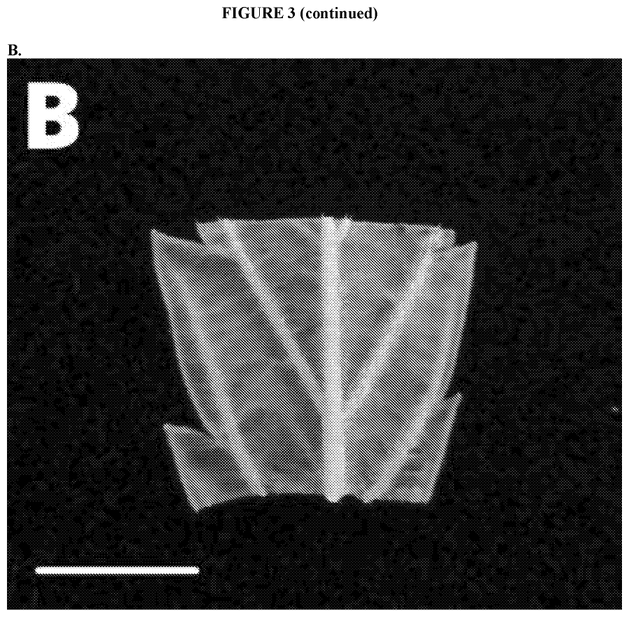

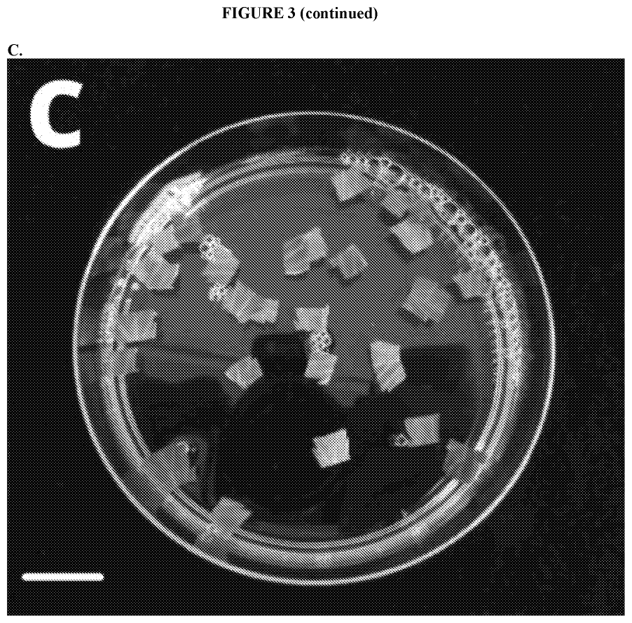

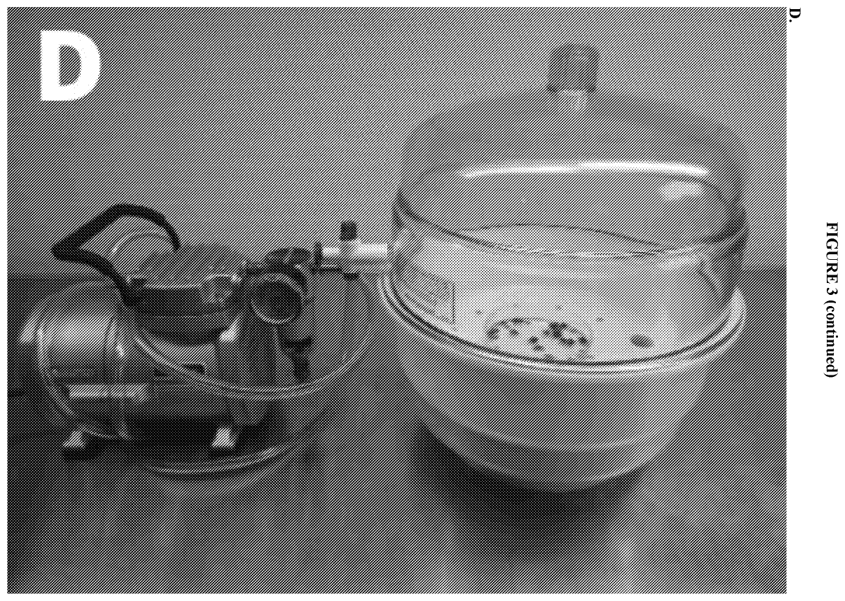

Agrobacterium strain, GV3101, was chosen for Agrobacterium-mediated transient expression in leaf discs of the cannabis plant. Recombinant pRNAi-GG vectors were transformed into GV3101 with appropriate selection. Agroinfiltration was achieved using vacuum infiltration on the excised cannabis leaf discs (FIG. 3) optimized for use with cannabis leaf material.

### Example 4—Silencing of Cannabinoid Biosynthesis Genes

Cannabis leaf discs were infiltrated with recombinant A. tumefaciens and incubated in a climate-controlled environment. To investigate the extent of downregulation of the cannabinoid biosynthesis genes, quantification of the transcript levels of THCAS, CBDAS and CBCAS was performed using qPCR. Each genes expression level was analyzed in three biological replicates and two technical replicates with gene primer pairs located upstream of the respective RNAi construct design.

Using the reference gene UBQ5 for normalization in all qPCR experiments, infiltrated leaf discs saw varying levels of down regulation in all cannabinoid biosynthesis genes compared to leaf discs infiltrated with disarmed Agrobacterium as a negative control. THCAS

Agroinfiltration with pRNAi-GG-THCAS downregulated THCAS, CBDAS and CBCAS transcript levels.

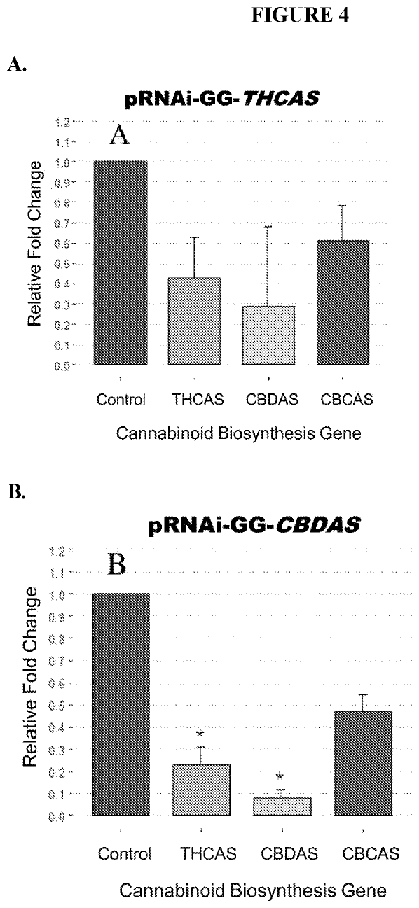

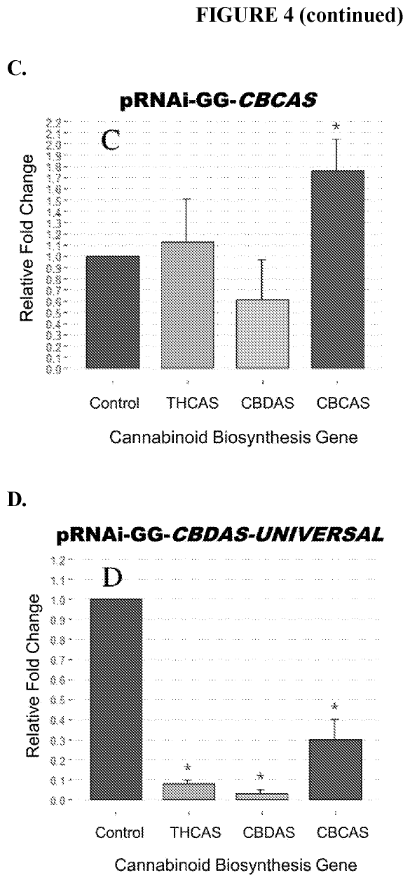

THCAS transcript levels were reduced by 57% relative to controls (FIG. 4A). Off-targeting of this vector construct resulted in a 71% reduction in CBDAS transcript levels and a 39% reduction in CBCAS transcript levels relative to controls.

### CBDAS

Agroinfiltration with pRNAi-GG-CBDAS downregulated THCAS, CBDAS and CBCAS transcript levels.

CBDAS transcript levels were reduced by 92% relative to controls (p<0.05; FIG. 4B). Off-targeting of this vector construct resulted in a 77% reduction in THCAS transcript levels (p<0.05) and a 53% reduction in CBCAS transcript levels relative to controls.

### CBCAS

Agroinfiltration with pRNAi-GG-CBCAS upregulated THCAS and CBCAS transcript levels, and downregulated CBDAS transcript levels.

CBCAS transcript levels were upregulated by 76% relative to controls (p<0.05, FIG. 4C). Off-targeting of this vector construct resulted in a 13% increase in THCA transcript levels and a 39% reduction in CBDAS transcript levels relative to controls.

### Universal

Agroinfiltration with pRNAi-GG-CBDAS-UNIVERSAL downregulated THCAS, CBDAS and CBCAS transcript levels.

CBCAS transcript levels were downregulated by 70% relative to controls (p<0.05, FIG. 4D). Off-targeting of this vector construct resulted in a 92% reduction in THCA transcript levels (p<0.05) and a 97% reduction in CBDAS transcript levels (p<0.05) relative to controls.

### Example 5—Cotyledon Callus Induction Frequency, Size and Weight Characteristics

321 transformed cotyledons were evaluated for callus induction and two-dimensional growth, with a subset of 84 cotyledons weighed across 4 weeks to measure growth parameters. A total of 60 control cotyledons, which were inoculated with a disarmed Agrobacterium, were also run in parallel. Seeds were germinated and cotyledons placed on CIM over 4 weekly intervals (Groups 1-4; Table 8). Cotyledons were placed on CIM at 3-days post-co-cultivation with the Agrobacterium culture, for 5 weeks, with size and weight and induction frequency being measured at week 2 (Table 8).

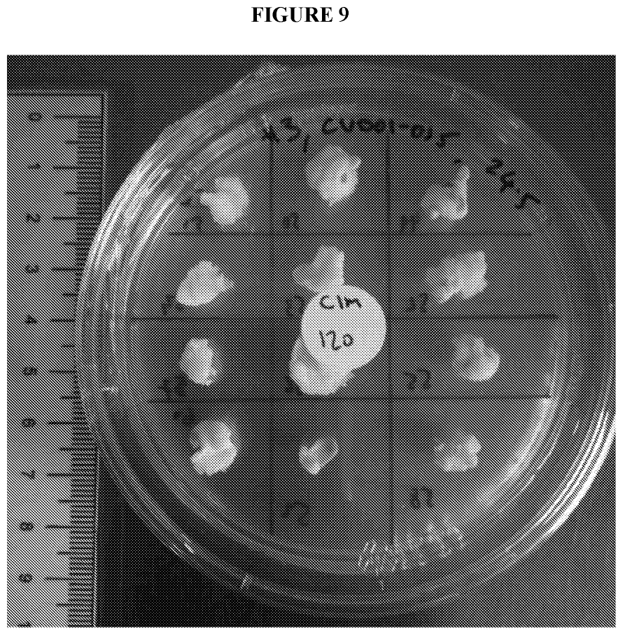

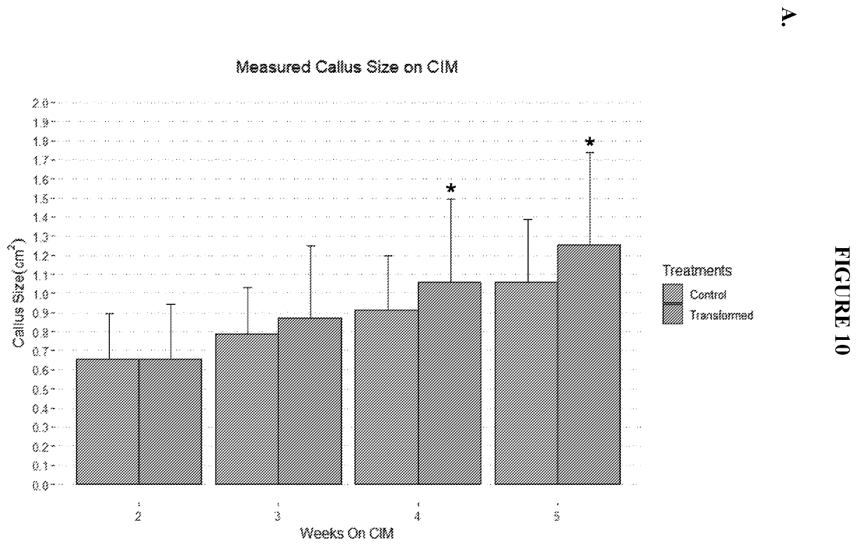

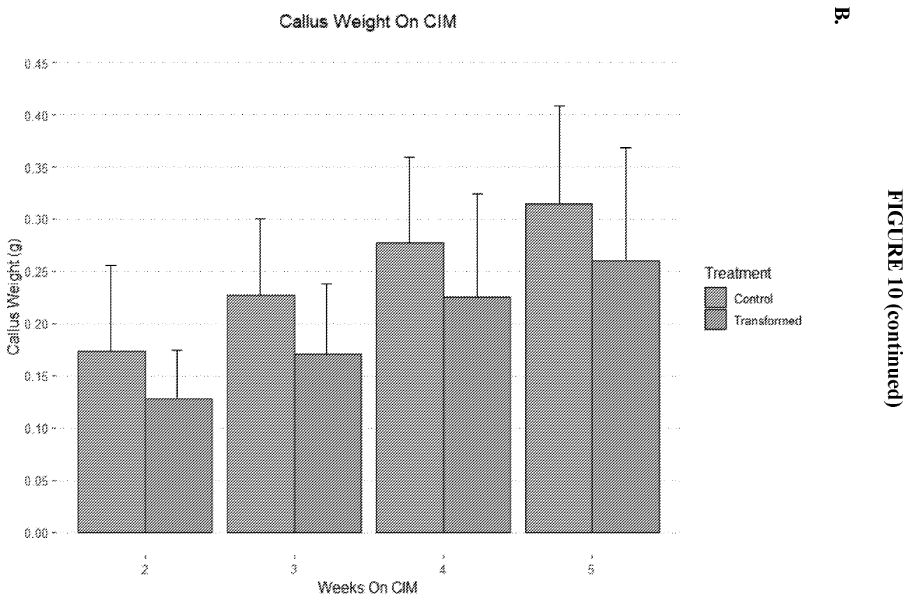

High callus induction rates were recorded across all groups, with 100% success measured in Groups 3 and 4. Slightly lower callus induction frequencies, 88.1% and 94.9% were recorded for Groups 1 and 2, respectively, with the average callus induction rate for transformed callus recorded at 95.76%. The control group had 93.3% success rate. Throughout the 5 weeks on CIM, 4 measurements for two-dimensional calli growth were recorded using ImageJ (FIG. 9).

Callus growth gradually increased across 4 weeks on CIM for treated and control groups (FIG. 10A). Group 2 calli size increased, on average, by 0.125 cm²/week. Group 3 had the largest average weekly increase, with 0.193 cm²/week. Groups 1 and 4 performed similarly with 0.143 and 0.138 cm²/week, respectively, with the average callus growth for the transformed cotyledons, being 0.146 cm²/week (Table 8). Measurements taken at weeks 4 and 5 on CIM, saw the transformed cotyledons significantly out-grow the control cotyledons (p<0.05; FIG. 10A). At weeks 4 and 5, the control group measured 0.915 cm² and 1.056 cm², respectively, with the transformed group measuring 1.082 cm² and 1.274 cm², respectively. Phenotypic response to callus induction (and transformation) saw the transformed cotyledons produce white, less dense callus compared to the control group, which produced more compact friable callus.

Group 1 (84 cotyledons) was used to measure callus weight from weeks 2-5 on CIM. The Group 1 cotyledons were weighed and recorded with an average of 0.033 g/week increase (Table 8), with sixty control cotyledons also weighed. The control callus had greater mass than the transformed cotyledons throughout the entire experiment (FIG. 10B), gaining on average 0.035 g/week (Table 8).

### Example 6—Callus Response to Different Regeneration Treatments and Transformation Frequency

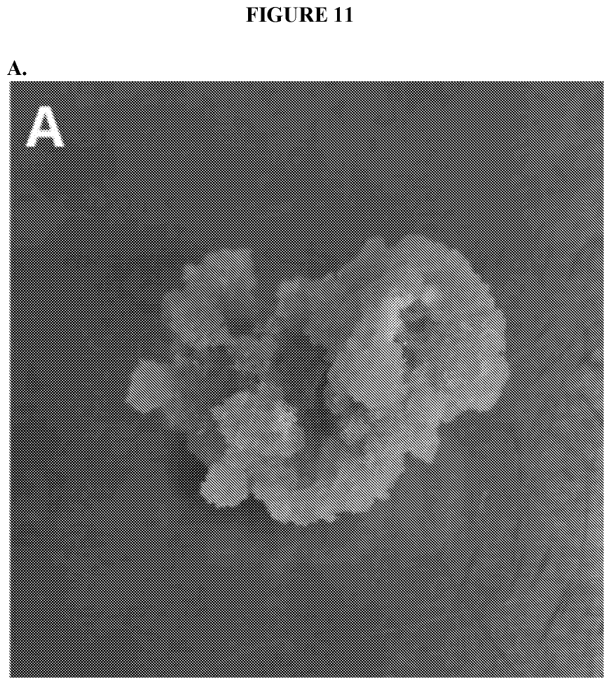

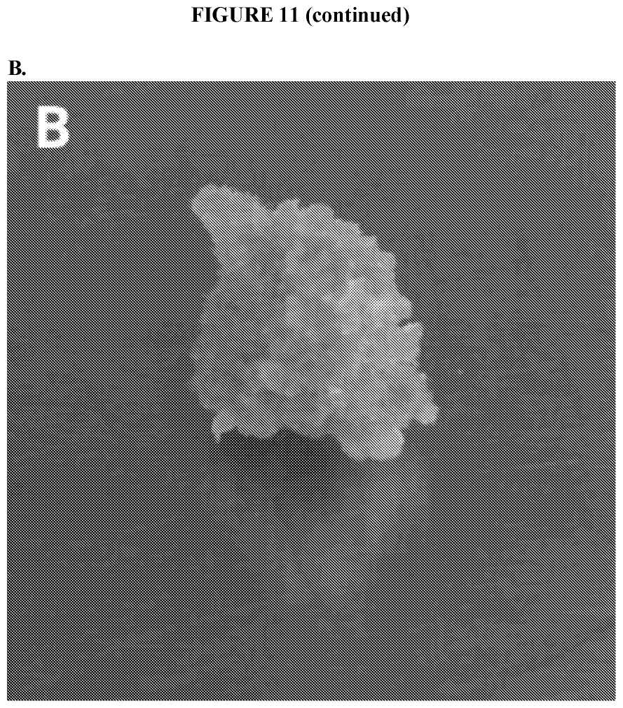

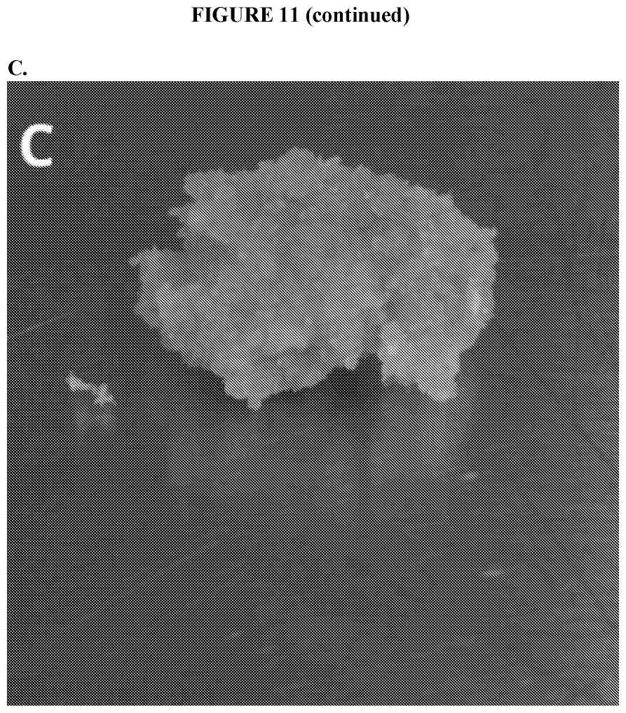

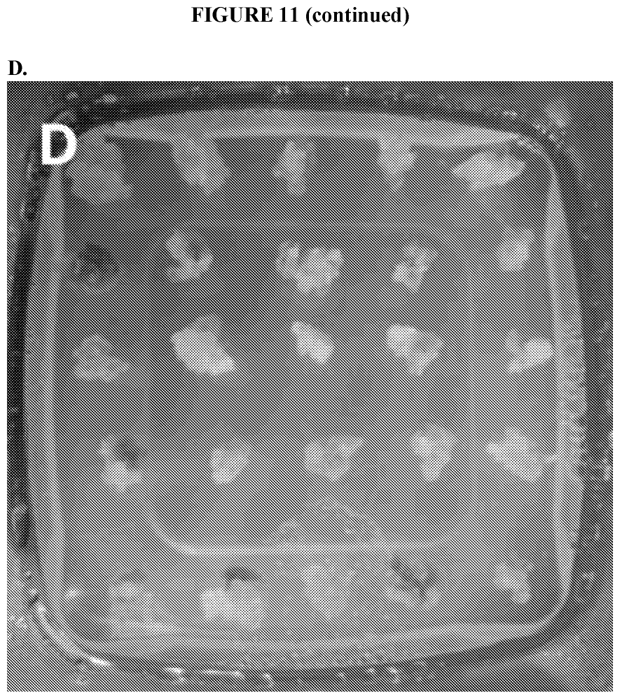

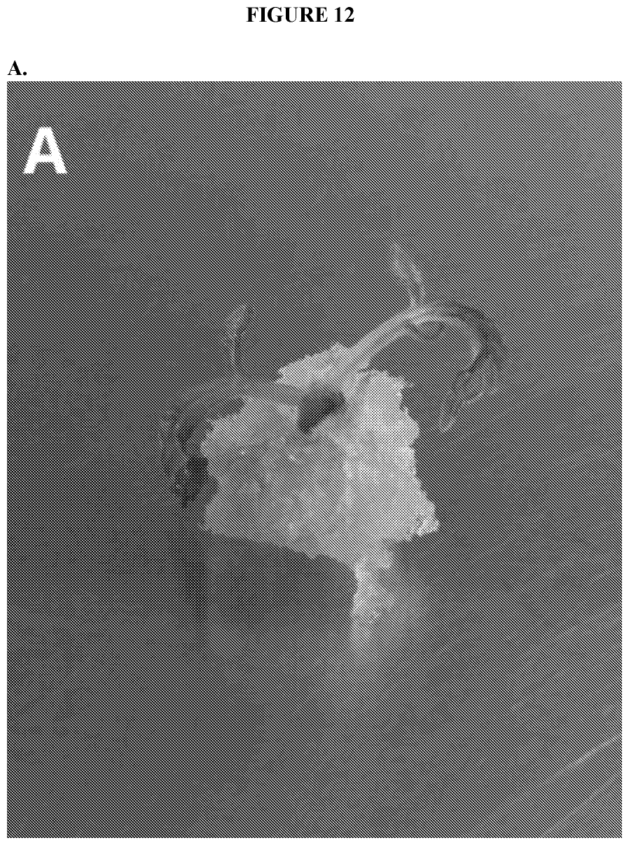

After 5 weeks on CIM, calli masses were transferred onto 2.5, 5 or 10 μM TDZ regeneration media to encourage shoot organogenesis. Transformed and control calli were randomly assigned different TDZ treatments and placed within the controlled environment room for 8 weeks. Phenotypic response to different TDZ concentrations on regeneration media of the transformed cotyledons showed no significant variation, with size, color and formation relatively uniform, suggesting that regardless of TDZ concentration, unorganized friable callus was produced with eventual plastid development present in green photosynthetic cells (FIG. 11A-D). Similarly, non-responding calli within the control group showed eventual plastid development in unorganized cellular masses with sizes and color comparable to transformed calli.

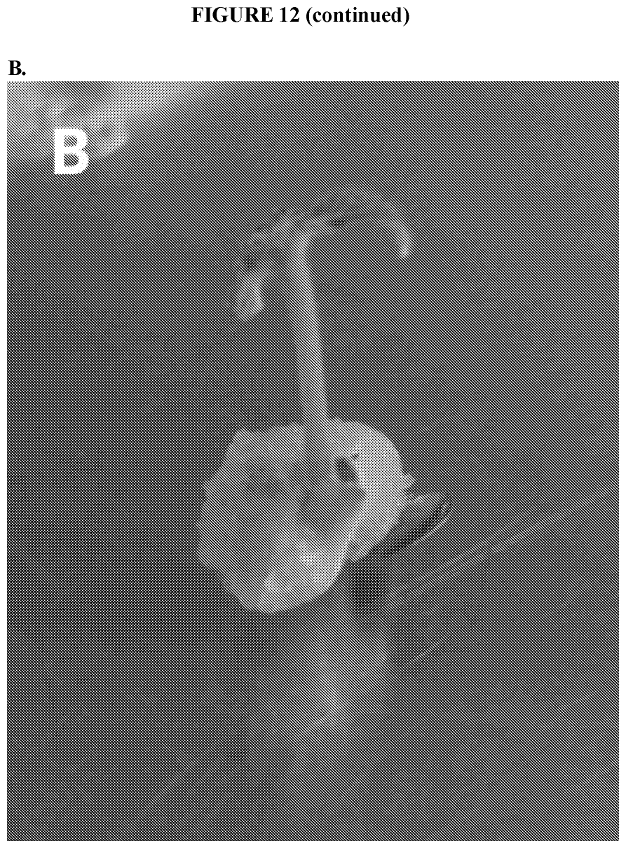

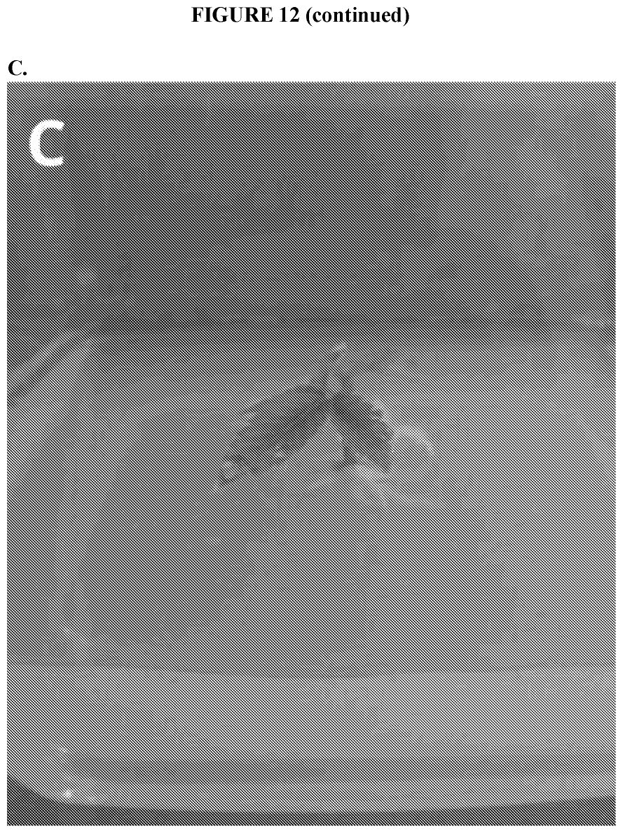

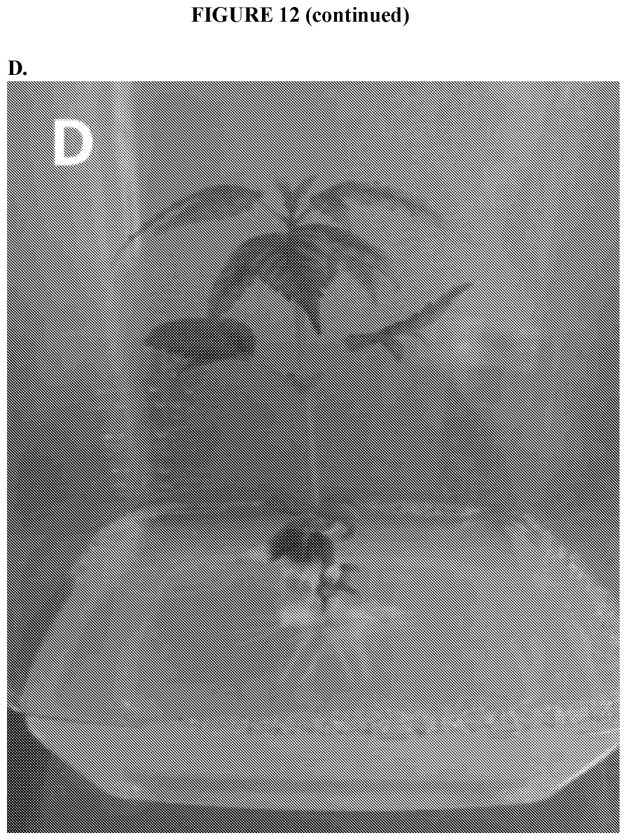

No regeneration was observed from transformed cotyledons treated with varying TDZ concentrations. However, leaf primordia were present in two transformed calli masses with regenerated leaves present, though organogenesis progress was not observed from these two calli masses. The control calli produced three regenerated shoots, with a single shoot produced from each of the three regeneration media compositions (FIG. 12A-D), resulting in a regeneration frequency within the control group of 5%.

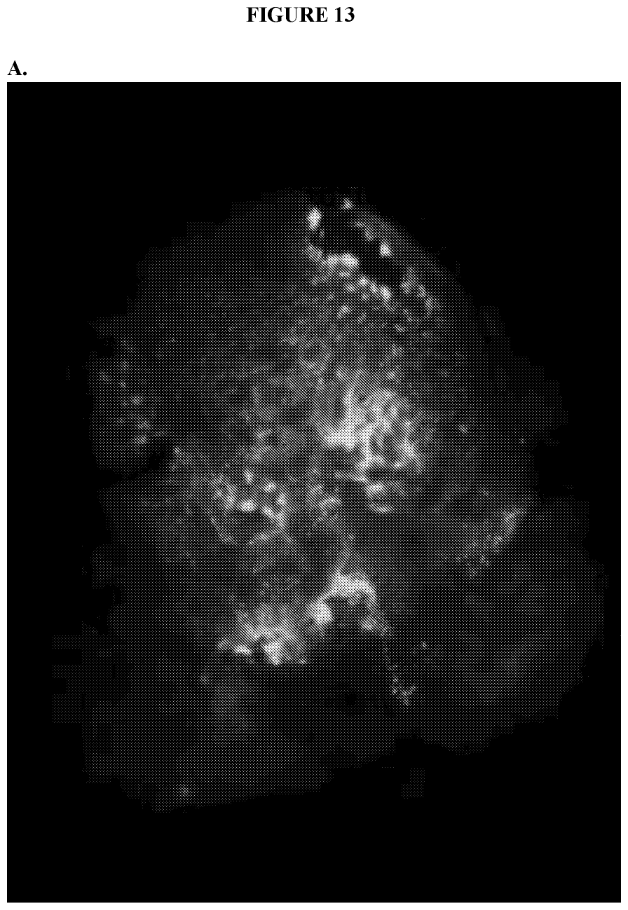

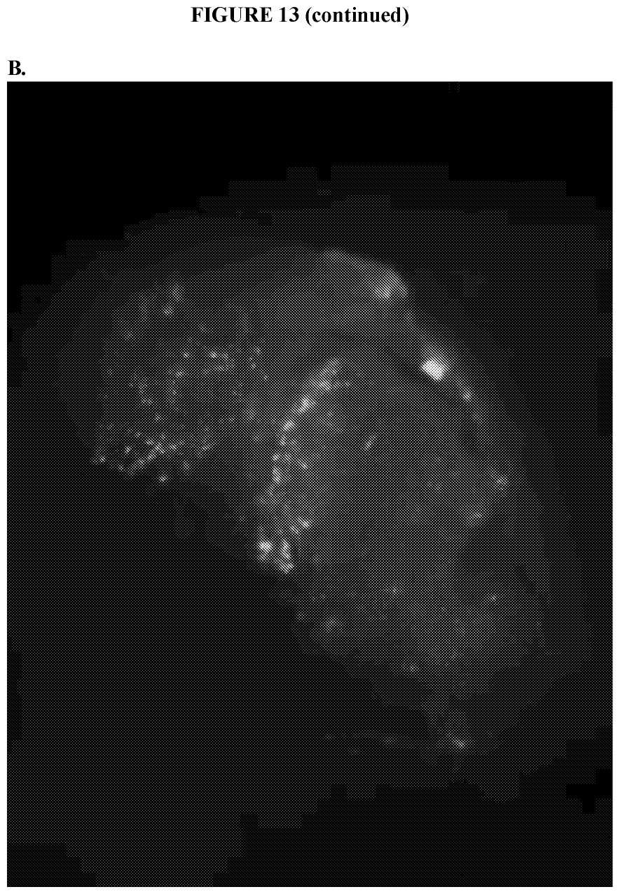

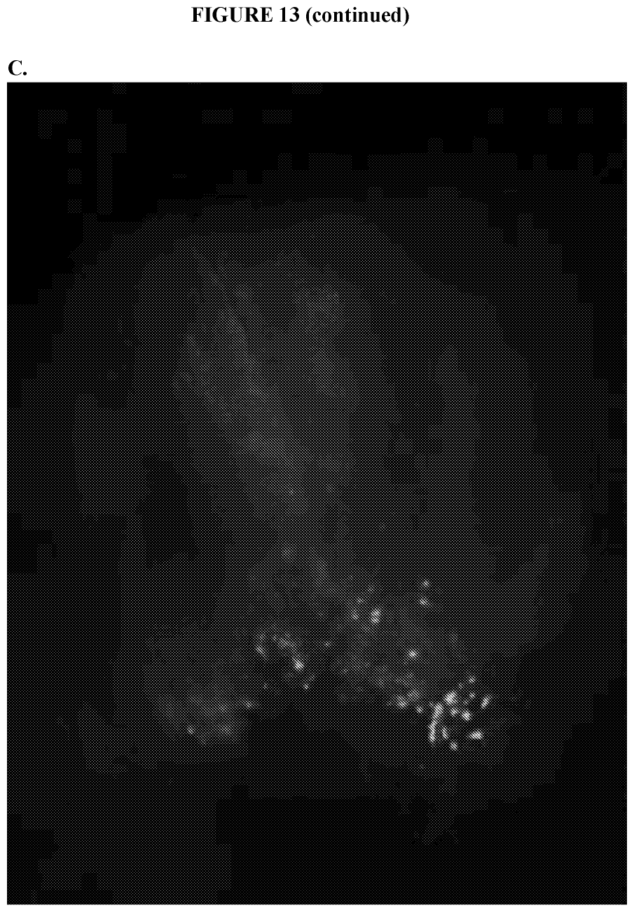

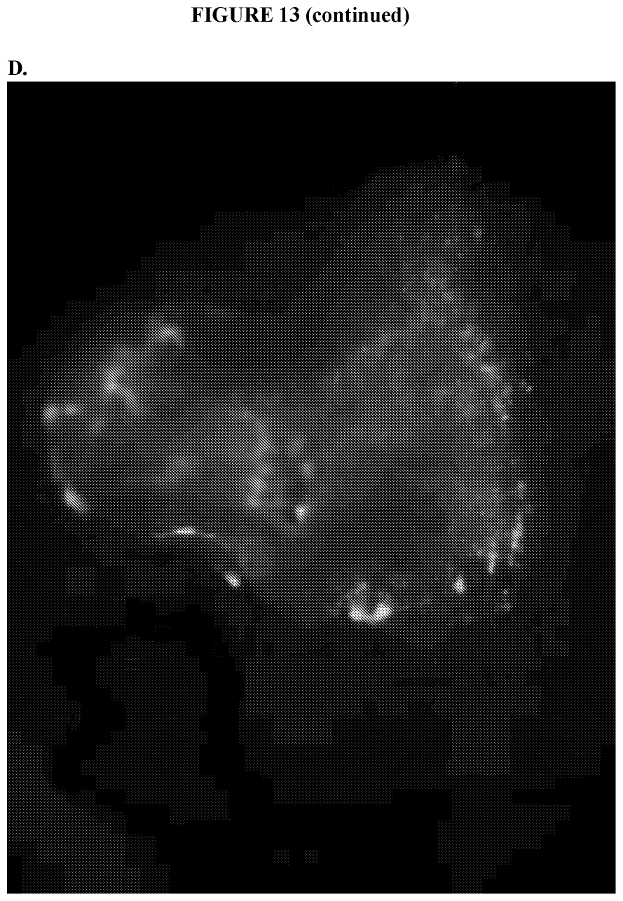

Transformation efficiency, defined as a single callus mass expressing GFP, was recorded at week 5 on regeneration media to evaluate GFP stability in transformed calli, with transformation scored as present/absent from GFP excitation under observation with a GFP filter. Similar GFP expression rates were observed across all 4 groups, with Group 2 having the lowest transformation efficiency of 57.6% and Group 3 achieving the highest efficiency with 69.2% (Table 9; FIG. 13). The average transformation efficiency observed across all groups was 62.3%.

### Example 7—Hypocotyl Rooting, Regeneration and Transformation Efficiency

304 transformed and 40 control (disarmed Agrobacterium) hypocotyls were evaluated for rooting, regeneration and transformation efficiency on hormone-free regeneration media post Agrobacterium co-transformation. Hypocotyls were placed on regeneration media for 4-8 weeks at weekly intervals (Groups 1-6) with efficiencies tabulated at week 4, respectively (Table 10).

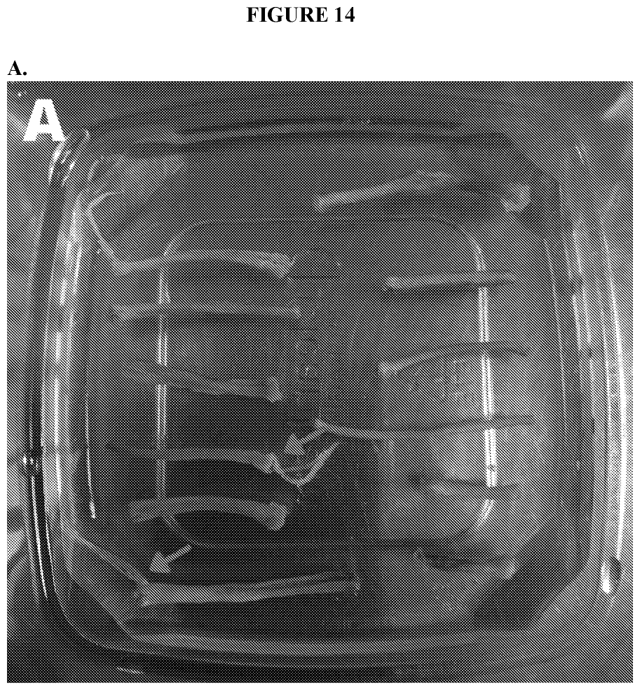

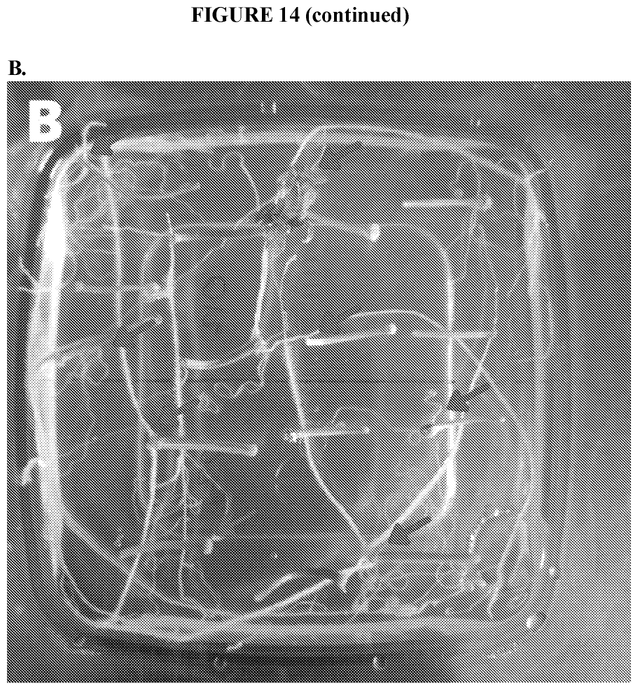

Spontaneous rooting efficiencies across transformed groups were lower compared to the control group. The lowest rooting efficiency, Group 1, achieved just 14.3% with Groups 2-6 only performing slightly better, with 19.1%, 22.2% and 21.2%, 23.5% and 23.3%, respectively. The average rooting efficiency for transformed hypocotyls was 20.6%. The control group achieved 47.5% efficiency (FIG. 14).

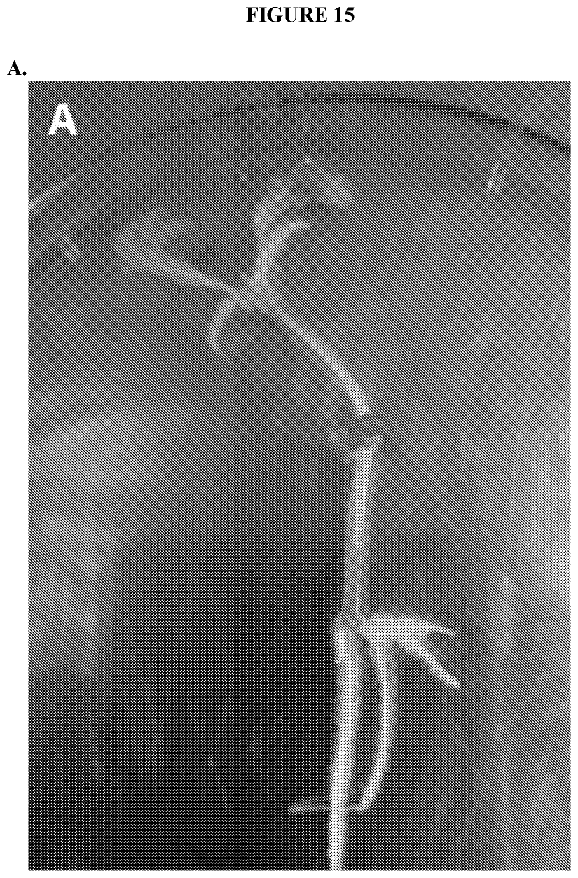

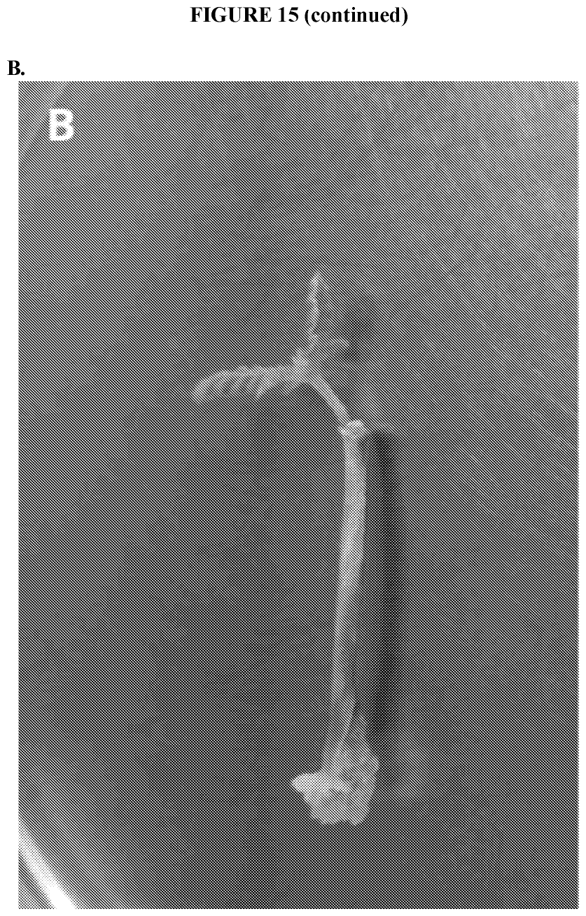

Hypocotyls with regenerated shoots was not achieved in Groups 1, 2, 4 and 6. Two hypocotyls responded with regenerated shoots in Group 3 (FIG. 15) and 1 regeneration event was observed in Group 5. Accordingly, an average regeneration response of 1.1% across all transformed hypocotyls. Comparatively, 8 regeneration events were observed in the controls, with an average regeneration response of 20% (Table 10; FIG. 15).

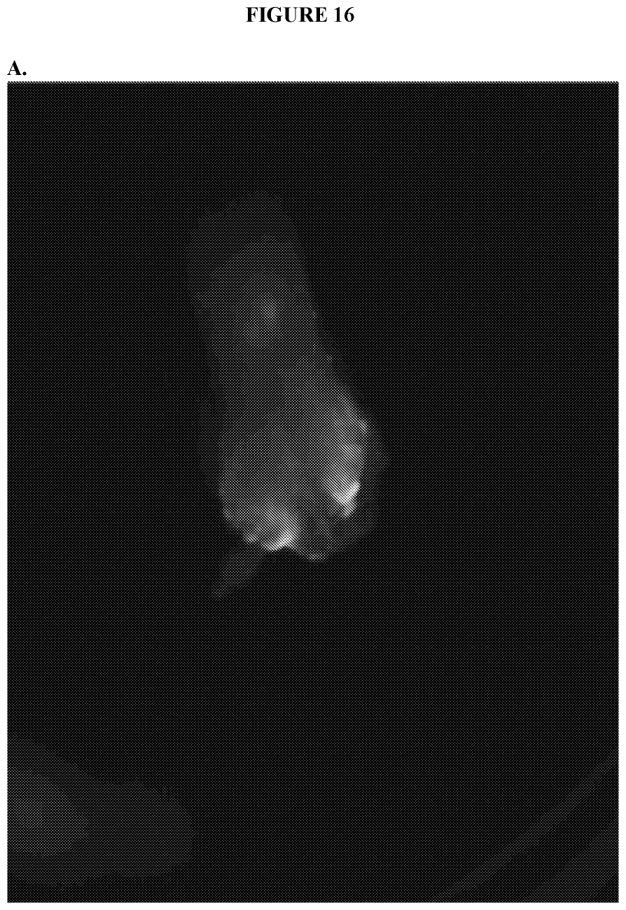

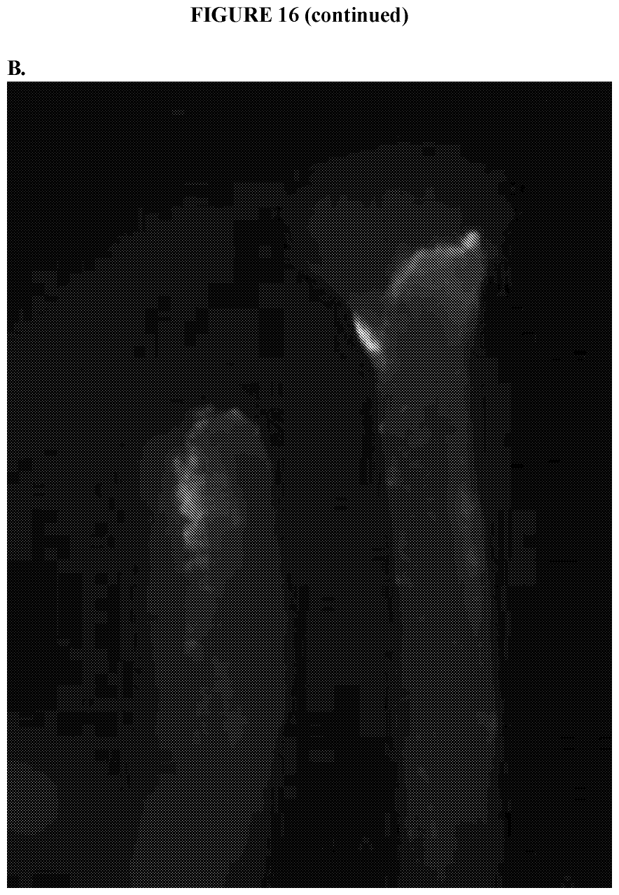

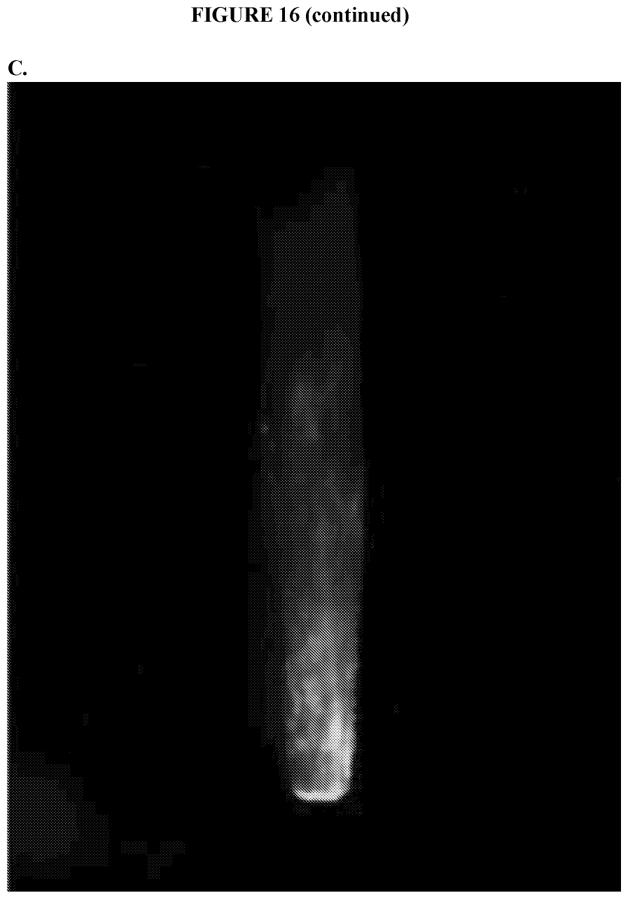

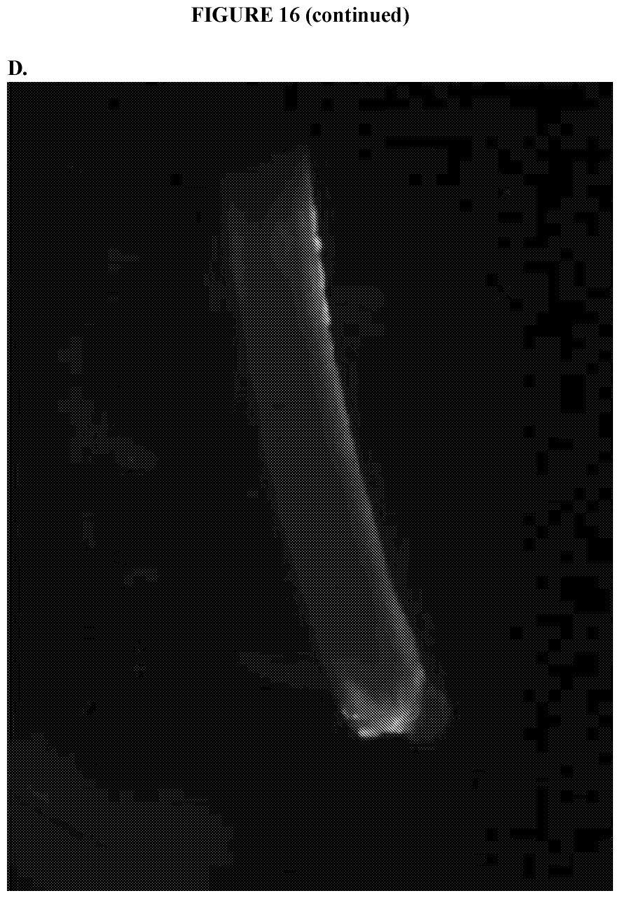

At 4 weeks post co-infiltration with Agrobacterium, the presence of GFP was recorded in transformed hypocotyls (FIG. 16), with GFP scored as present/absent from GFP excitation under observation with a GFP filter. Group 1 achieved the highest average rate of transformation, with 55.4% efficiency, followed closely by Group 2 with 55.3%. Group 3 achieved the lowest efficiency, with 40.0%. Groups 4, 5 and 6 achieved very similar results, with 46.2%, 45.1% and 46.5%, respectively, with the average transformation efficiency across all groups of 48.1% (Table 10).

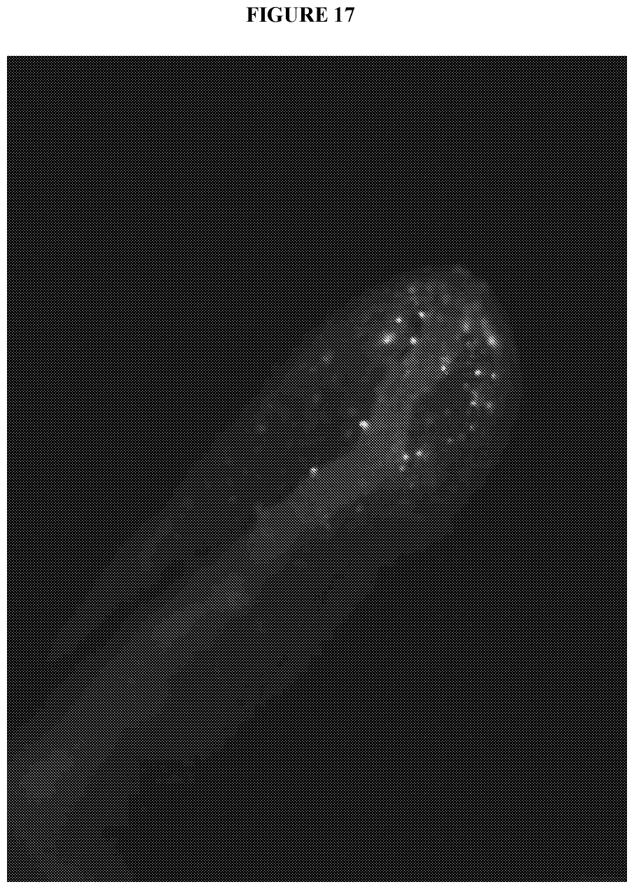

A total of 3 regenerated shoots from transformed hypocotyls were present, though only 1 acclimatized to rooting media. The acclimatized regenerated hypocotyl was imaged for stable GFP fluorescence. Four selected leaves contained GFP excitation within the leaf tip apex and leaf mid-veins (FIG. 17).

### Example 8—RT-qPCR Data of Cotyledon Calli

Cotyledon callus transcript levels of targeted genes from mRNA in control and transformed samples were analyzed for alterations in transcript level. In total, RNA was extracted from 12 control and 24 transformed cotyledon callus and analyzed using RT-qPCR with specific primers (Table 4) targeting THCAS, CBDAS, CBCAS using UBQ5 as an internal housekeeping reference gene for normalization. Due to the outcrossing nature of cannabis, cotyledon calli are not genetically uniform with variance in gene expression from targeted genes. Therefore, to best normalize transcript levels, equal concentrations of RNA from each control were mixed to produce a pooled sample which were used for normalization of gene expression, which was run along with RNA from individual control callus. The pooled RNA expression levels were used for subsequent calculations of relative gene expression of THCAS, CBDAS and CBCAS using the 2^{-ΔΔCt} method. Extracted RNA from cotyledon masses, after 8 weeks on regeneration media, has shown significant down and upregulation of transcript levels, within each transformed callus, with the exception of callus #21. Significant downregulation of all three genes was recorded for callus masses 1-4, 9 and 17. Significant upregulation in at least one of the three genes was recorded for callus masses 5, 7, 10, 12, 14, 16, 19, 20, 22 and 24. Significant upregulation of THCAS was substantially more prevalent compared to CBDAS and CBCAS. CBDAS was the most substantially altered with significant downregulation recorded in 20 callus masses (FIG. 18). Co-transformation of cotyledons with a GFP vector (FIG. 19), measured by a significant (p<0.05) modified expression profile and GFP excitation under fluorescence, saw a co-transformation rate of 75% (18/24).

### DISCUSSION

Vacuum infiltration was achieved in leaf discs of a cannabis cultivar to significantly reduce the relative expression of cannabinoid biosynthesis genes: THCAS, CBDAS and CBCAS.

The design and optimization of RNAi constructs as described herein leverages the off-target effects of RNAi constructs to specifically target cannabinoid biosynthesis genes simultaneously. As shown herein, the use of RNAi constructs designed to target any one or more or all of the cannabinoid biosynthesis genes THCAS, CBDAS and CBCAS can reduce the transcript levels for each of THCAS, CBDAS and CBCAS.

Highly homologous (>90%) gene sequences, when amplified and used in RNAi, will produce siRNA are likely to have significant off-targeting. This has been demonstrated by the inventors', in that siRNA predicted from the amplified THCAS sequence were more effective in downregulating the CBDAS transcripts, comparatively, to THCAS and CBCAS, which are more highly sequence homologous (>96%) than CBDAS is to THCAS (92%). The increased downregulation of CBDAS may be the related to the number of copies of the gene in the cannabis genome (i.e., potentially 5 functional copies). This increase in copy number will greatly affect RNAi specificity and will result in a higher number of off-targeting sites.

siRNA efficacy may also be optimized by using shorter PCR products as the input sequence for siRNA design. For example, pRNAi-GG-CBDAS-UNIVERSAL, comprises a 247 bp fragment, which produced significant (p<0.05) reduction in THCAS, CBDAS and CBCAS (FIG. 4). Increased efficacy of shorter dsRNA fragments has previously been confirmed in potato (He et al., 2020, Journal of Experimental Botany, 71(9): 2670-2677), with evidence supporting shorter dsRNA length resulting in increased levels of insecticidal protection compared to the larger RNAi constructs investigated. Other design considerations that may be relevant to improved efficacy of the siRNA include the amount of mismatches and the identity of the matched nucleotides (Du et al., 2005, Nucleic Acids Research, 33(5): 1671-1677). For example, adenine and cytosine, along with G:U wobble base pair mismatches are silenced with equal efficiency.

Using RNAi to significantly downregulate the medicinally important cannabinoid biosynthesis genes can be achieved using Agrobacterium. The drawback from using RNAi to target these genes is the unintended off-targeting, resulting in silencing of the other highly homologous genes. The use of this agroinfiltration RNAi approach, generating a transformational event resulting in a designer cannabis strain with significantly reduced THC, CBD and CBC concentrations is possible. The decreased gene expression will potentially lead to a dramatic increase in the precursor CBG, which is currently found in minute concentrations, comparatively (Stack et al., 2021, GCB Bioenergy, 13(4): 546-561).

It has therefore been surprisingly demonstrated that cannabinoid biosynthesis genes can be altered using RNAi vectors comprising a nucleotide sequence encoding siRNA targeting one or more or all of a THCAS gene, at least one CBDAS gene and at least one CBCAS gene.

The selection and use such siRNA has been further reduced to practice in the production of stably transformed C. sativa tissues, which is important in the development of medicinal cannabis. In particular, transformed calli showed stable integration of the RNAi vector, with significant down and upregulation of the cannabinoid biosynthesis genes observed across the transformed calli.

The effect of siRNA off-targeting on transcript levels in the stably transformed calli as compared to the transient expression of the RNAi vectors in the leaf explant may be explained, in part, by the different explant tissue type. For example, the calli contained lower transcript levels as compared to the leaf explant. This reduction in baseline transcript level may be attributed to the lack of developed organelles and biological structures in callus. Further, given the high level of homology within the targeted gene sets, upregulation of one or more of the cannabinoid biosynthesis genes may be due to non-specific upregulation (Zirpel et al., 2018, Journal of Biotechnology, 284: 17-26; Fulvio et al., 2021, Plants, 10(9): 1857) or through transcription-translation interference causing upregulation, which has been demonstrated to occur using microRNAs and in mammalian siRNA systems (Vasudevan, 2012, Wiley Interdisciplinary Reviews RNA, 3(3): 311-30; Portnoy et al., 2011, Wiley Interdisciplinary Reviews RNA, 2(5): 748-60). Although downregulation of all of the cannabinoid biosynthesis genes were not recorded across all transformed calli, cleaving of mRNA by siRNA may still be occurring, resulting in the upregulation of these genes due to their innate ability to synthesize non-specific cannabinoids as has been previously shown (Matchett-Oates et al., 2021, Frontiers in Plant Science, 12: 773474; Zirpel et al., 2018, supra; and Fulvio et al., 2021, supra).

It has therefore been surprisingly demonstrated that cannabinoid biosynthesis genes can be altered using stably expressed RNAi constructs in tissue types that are capable of regenerating cannabis plants with altered expression of cannabinoid biosynthesis genes.

TABLE 1
Cannabinoids and their properties.

| | | Chemical properties/ |
|---|---|---|
| Name | Structure | [M + H]⁺ ESI MS |
| Δ9- tetrahydrocannabinol (THC) | 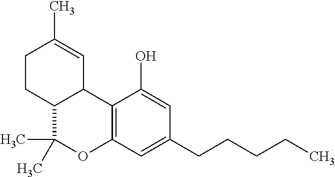 | Psychoactive, decarboxylation product of THCA m/z 315.2319 |
| Δ9- tetrahydrocannabinolic acid (THCA/THCA-A) | 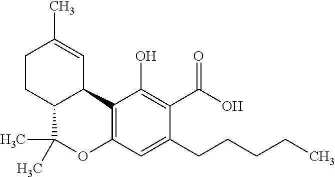 [SMILES: CCCCCc1cc2c(c(O)c1C(=O)O)[C@@H]1C=C(C)CC[C@H]1C(C)(C)O2] | m/z 359.2217 |
| cannabidiol (CBD) | 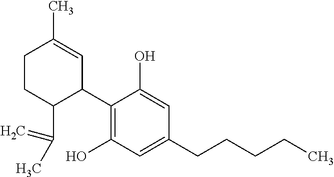 [SMILES: C=C(C)C1CCC(C)=CC1c1c(O)cc(CCCCC)cc1O] | decarboxylation product of CBDA m/z 315.2319 |
| cannabidiolic acid (CBDA) | 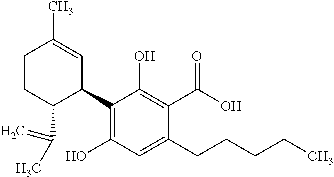 [SMILES: C=C(C)[C@@H]1CCC(C)=C[C@H]1c1c(O)cc(CCCCC)c(C(=O)O)c1O] | m/z 359.2217 |
| cannabigerol (CBG) | 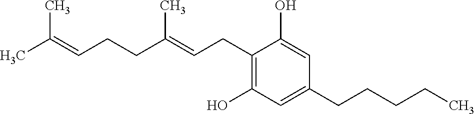 [SMILES: CCCCCc1cc(O)c(C/C=C(\C)CCC=C(C)C)c(O)c1] | Non-intoxication, decarboxylation product of CBGA m/z 317.2475 |
| cannabigerolic acid (CBGA) | 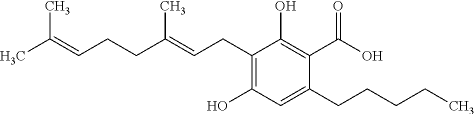 [SMILES: CCCCCc1cc(O)c(C/C=C(\C)CCC=C(C)C)c(O)c1C(=O)O] | m/z 361.2373 |
| cannabichromene (CBC) | 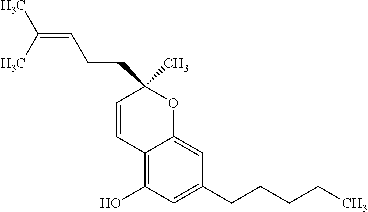 [SMILES: CCCCCc1cc(O)c2c(c1)O[C@](C)(CCC=C(C)C)C=C2] | Non-psychotropic, converts to cannabicyclol upon light exposure m/z 315.2319 |
| cannabichromene acid (CBCA) | 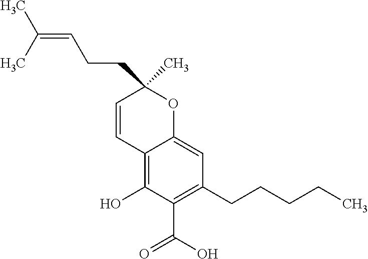 [SMILES: CCCCCc1cc2c(c(O)c1C(=O)O)C=C[C@@](C)(CCC=C(C)C)O2] | m/z 359.2217 |
| cannabicyclol (CBL) | 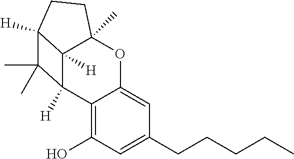 | Non-psychoactive, 16 isomers known. Derived from non-enzymatic conversion of CBC m/z 315.2319 |
| cannabinol (CBN) | 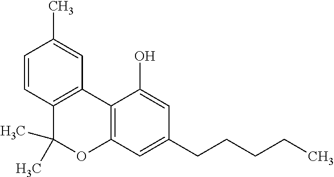 [SMILES: CCCCCc1cc(O)c2c(c1)OC(C)(C)c1ccc(C)cc1-2] | Likely degradation product of THC m/z 311.2006 |
| cannabinolic acid (CBNA) | 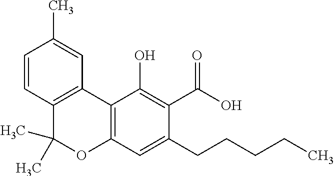 [SMILES: CCCCCc1cc2c(c(O)c1C(=O)O)-c1cc(C)ccc1C(C)(C)O2] | m/z 355.1904 |
| tetrahydrocannabivarin (THCV) | 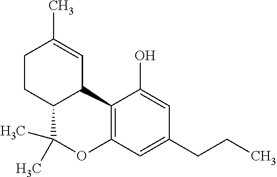 | decarboxylation product of THCVA m/z 287.2006 |
| tetrahydrocannabivarinic acid (THCVA) | 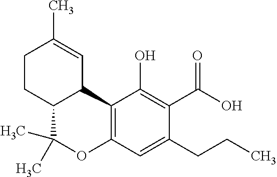 | m/z 331.1904 |
| cannabidivarin (CBDV) | 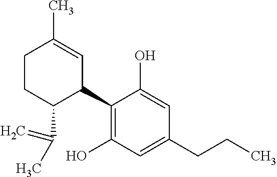 | m/z 287.2006 |
| cannabidivarinic acid (CBDVA) | 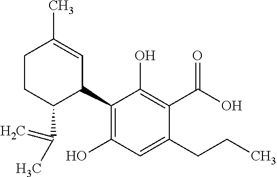 | m/z 331.1904 |
| Δ8- tetrahydrocannabinol (d8-THC) | 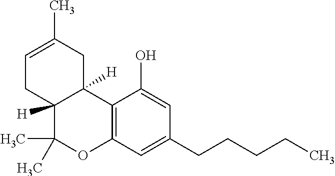 [SMILES: CCCCCc1cc(O)c2c(c1)OC(C)(C)[C@@H]1CC=C(C)C[C@@H]21] | m/z 315.2319 |

TABLE 2
Primers for cDNA amplification

| | | SEQ | |
|---|---|---|---|
| | | ID | |
| Target | Direction | NO: | Sequence |
| THCAS | Forward | 323 | accaggtctcaggagAACTATTTTATGCTCTAAGAAAGT |
| | Reverse | 324 | accaggtctcatcgt TCATGACTCACTTCATAACAAA |
| CBDAS | Forward | 325 | accaggtctcaggag AAGTCCCATTTGTTATAGTAGA |
| | Reverse | 326 | accaggtctcatcgt GAGATACATGAGCTTGTCAA |
| CBCAS | Forward | 327 | accaggtctcaggag GGCCAGTATTCTCTGCTC |
| | Reverse | 328 | accaggtctcatcgt CACGACTCACTTCAGAACTAG |
| Universal | Forward | 329 | accaggtctcaggag CCGGAGCTACCCTT |
| | Reverse | 330 | accaggtctcatcgtGGCTATACGTGGTGG |

TABLE 3
Flanking primers for recombinant E. coli

| | | SEQ | |
|---|---|---|---|
| | | ID | |
| Target | Direction | NO: | Sequence |
| Arm#1 | Forward | 331 | CATTTGGATTGATTACAGTTGG |
| | Reverse | 332 | ACCCAACTTAATCGCCTT |
| Arm#2 | Forward | 333 | CGAATCTCAAGCAATCAAGC |
| | Reverse | 334 | ATTTTGAGGCATTTCAGTCA |

TABLE 4
Primer sequences for qPCR

| | | SEQ | |
|---|---|---|---|
| | | ID | |
| Target | Direction | NO: | Sequence |
| THCAS | Forward | 339 | AATTTTTCATGGTGGAGTGGAT |
| | Reverse | 340 | AAAATTTACAACACCACTGTAGAA |
| CBDAS | Forward | 341 | CAGTTACTTCTCCTCCATTTTCC |
| #1 | Reverse | 342 | ATAGTATCAATCCAGCTCAACT |
| CBDAS | Forward | 345 | GGAAAACTGAAGAGAAGTAAGTG |
| #2 | Reverse | 346 | AATTGAGCTGGATTGATACTAT |
| CBCAS | Forward | 343 | ACCTGGGAGAAGCAAGAAG |
| | Reverse | 344 | GGATTAGTTTTTCCTAAATCAAGG |

TABLE 5
Analysis of cannabinoid biosynthesis genes with PCR amplification,
copy number and siRNA prediction information

| | | Primer | | | |
|---|---|---|---|---|---|
| | Copy | pairs | | | Number of |
| | number/ | used for | SEQ | Product | predicted |
| Target | homologs | amplification | ID NO: | size (bp) | siRNA |
| THCAS | 1 | Forward | 323 | 603 | 93 |
| | | Reverse | 324 | | |
| CBDAS | 9 | Forward | 325 | 442 | 70 |
| | | Reverse | 326 | | |
| CBCAS | 3 | Forward | 327 | 606 | 95 |
| | | Reverse | 328 | | |
| Universal | CBDAS- | Forward | 329 | 247 | 38 |
| | truncated-4 | Reverse | 330 | | |
| | homolog | | | | |

| | | CBCAS | CBCAS | CBCAS- | CBDAS | CBDAS | CBDAS- |
|---|---|---|---|---|---|---|---|
| | THCAS | #1 | #2 | t | #1 | #2 | l#1 |
| THCAS | | | | | | | |
| CBCAS | 96.31 | | | | | | |
| #1 | | | | | | | |
| CBCAS | 96.23 | 99.93 | | | | | |
| #2 | | | | | | | |
| CBCAS- | 95.88 | 99.90 | 99.79 | | | | |
| t | | | | | | | |
| CBDAS | 93.10 | 92.38 | 92.29 | 91.83 | | | |
| #1 | | | | | | | |
| CBDAS | 93.10 | 92.38 | 92.29 | 91.83 | 100.00 | | |
| #2 | | | | | | | |
| CBDAS- | 92.98 | 92.28 | 92.20 | 91.71 | 99.51 | 99.51 | |
| l#1 | | | | | | | |
| CBDAS- | 92.60 | 92.03 | 91.96 | 91.44 | 99.51 | 99.51 | 99.44 |
| l#2 | | | | | | | |
| CBDAS- | 92.37 | 91.64 | 91.56 | 91.03 | 99.49 | 99.49 | 99.26 |
| l#3 | | | | | | | |
| CBDAS- | 92.19 | 92.74 | 92.74 | 52.19 | 98.96 | 99.96 | 98.44 |
| t#1 | | | | | | | |
| CBDAS- | 72.86 | 71.35 | 71.28 | 61.84 | 74.38 | 74.38 | 74.80 |
| t#2 | | | | | | | |
| CBDAS- | 86.39 | 86.66 | 86.66 | 86.42 | 87.01 | 87.01 | 86.90 |
| t#3 | | | | | | | |
| CBDAS- | 67.78 | 66.70 | 66.60 | 62.03 | 66.14 | 66.14 | 67.17 |
| t#4 | | | | | | | |

| | CBDAS- | CBDAS- | CBDAS- | CBDAS- | CBDAS- | CBDAS- |
|---|---|---|---|---|---|---|
| | l#2 | l#3 | t#1 | t#2 | t#3 | t#3 |
| THCAS | | | | | | |
| CBCAS | | | | | | |
| #1 | | | | | | |
| CBCAS | | | | | | |
| #2 | | | | | | |
| CBCAS- | | | | | | |
| t | | | | | | |
| CBDAS | | | | | | |
| #1 | | | | | | |
| CBDAS | | | | | | |
| #2 | | | | | | |
| CBDAS- | | | | | | |
| l#1 | | | | | | |
| CBDAS- | | | | | | |
| l#2 | | | | | | |
| CBDAS- | 99.11 | | | | | |
| l#3 | | | | | | |
| CBDAS- | 98.96 | 98.54 | | | | |
| t#1 | | | | | | |
| CBDAS- | 76.62 | 75.72 | 98.44 | | | |
| t#2 | | | | | | |
| CBDAS- | 86.90 | 86.87 | 84.74 | 56.28 | | |
| t#3 | | | | | | |
| CBDAS- | 67.17 | 67.39 | 90.00 | 82.72 | 57.83 | |
| t#4 | | | | | | |

TABLE 7
Off-targeting frequency in each cannabinoid
gene from generated siRNA in each vector

| | RNAi Vector | | | |
|---|---|---|---|---|
| Cannabinoid | pRNAi- | pRNAi- | pRNAi- | pRNAi- |
| biosynthesis | GG- | GG- | GG- | GG-CBDAS- |
| genes | THCAS | CBDAS | CBCAS | UNIVERSAL |
| THCAS | 93 | 18 | 53 | 0 |
| CBCAS#1 | 53 | 16 | 95 | 0 |
| CBCAS#2 | 53 | 16 | 95 | 0 |
| CBCAS-trun | 51 | 14 | 86 | 0 |
| CBDAS#1 | 18 | 64 | 16 | 5 |
| CBDAS#2 | 18 | 64 | 16 | 5 |
| CBDAS-like#1 | 16 | 70 | 16 | 5 |
| CBDAS-like#2 | 23 | 54 | 18 | 5 |
| CBDAS-like#3 | 17 | 54 | 12 | 5 |
| CBDAS-trun#1 | 0 | 0 | 0 | 0 |
| CBDAS-trun#2 | 19 | 60 | 16 | 5 |
| CBDAS-trun#3 | 0 | 0 | 0 | 1 |
| CBDAS-trun#3 | 6 | 15 | 5 | 38 |

TABLE 8
Measured cotyledon characteristics across 5 weeks on CIM

| | Callus | Calli size | Calli weight |
|---|---|---|---|
| | induction | growth | increase |
| Cotyledon callus | (%) | (avg. cm²/week) | (avg. g/week) |
| Group 1 (84 samples) | 88.10 | 0.142 | 0.033 |
| Group 2 (59 samples) | 94.92 | 0.125 | |
| Group 3 (78 samples) | 100 | 0.193 | |
| Group 4 (120 samples) | 100 | 0.138 | |
| Average (transformed) | 95.76 | 0.146 | |
| Control (60 samples) | 93.33 | 0.100 | 0.035 |

TABLE 9
Transformation efficiency (defined as a single callus
mass expressing GFP) in cotyledons after 5 weeks on CIM

| | Transformation |
|---|---|
| Cotyledon callus | efficiency (%) |
| Group 1 | 63.1 |
| Group 2 | 57.6 |
| Group 3 | 69.2 |
| Group 4 | 59.2 |
| Average | 62.3 |

TABLE 10
Hypocotyl rooting, regeneration and transformation
efficiencies across Groups and Controls

| | Average | Average | |
|---|---|---|---|
| | Rooting | Shoot | Transformation |
| | Induction | Regeneration | Efficiency |
| Group 1 (56 samples) | 8/56 (14.3%) | 0 | 31/56 (55.4%) |
| Group 2 (47 samples) | 9/47 (19.1%) | 0 | 26/47 (55.3%) |
| Group 3 (45 samples) | 10/45 (22.2%) | 2/45 (4.4%) | 18/45 (40%) |
| Group 4 (52 samples) | 11/52 (21.2%) | 0 | 24/52 (46.2%) |
| Group 5 (51 samples) | 12/51 (23.5%) | 1/51 (2%) | 23/51 (45.1%) |
| Group 6 (43 samples) | 10/43 (23.3%) | 0 | 20/43 (46.5% |
| Average (transformed) | 20.6% | 1.1% | 48.1% |
| Control (40 samples) | 19/40 (47.5%) | 8/40 (20%) | — |

1-47. (canceled)

48. A cannabis cell comprising a targeted genetic modification that alters the expression of one or more cannabinoid biosynthesis genes, wherein the genetic modification is targeted to one or more, or all of:
a) a region of at least one CBDAS gene comprising a nucleotide sequence corresponding to any one of SEQ ID NOs: 18-26, or a or a nucleotide sequence which is at least 90% identical to the nucleotide sequence of SEQ ID NOs: 18-26;
b) a region of at least one CBCAS gene comprising a nucleotide sequence corresponding to any one of SEQ ID NOs: 15-17, or a or a nucleotide sequence which is at least 90% identical to the nucleotide sequence of SEQ ID NOs: 15-17; and
c) a region of a THCAS gene comprising a nucleotide sequence corresponding to SEQ ID NO: 14, or a or a nucleotide sequence which is at least 90% identical to the nucleotide sequence of SEQ ID NO: 14,
and wherein the cell is characterized by altered expression of the one or more cannabinoid biosynthesis genes relative to a cannabis cell that does not comprise the genetic modification.

49. The cannabis cell of claim 48, wherein the genetic modification is targeted to:
a) a region of at least one CBDAS gene comprising a nucleotide sequence corresponding to nucleotides 17 to 458 of SEQ ID NOs: 18 or 19, nucleotides 53 to 494 of SEQ ID NO: 20, nucleotides 191 to 632 of SEQ ID NOs: 21 or 22, nucleotides 155 to 595 of SEQ ID NO: 24, nucleotides 1 to 149 of SEQ ID NO: 25, or nucleotides 77 to 518 of SEQ ID NO: 26;
b) a region of at least one CBCAS gene comprising a nucleotide sequence corresponding to nucleotides 102 to 707 of SEQ ID NOs: 15, 16 or 17; and
c) a region of a THCAS gene comprising a nucleotide sequence corresponding to nucleotides 105 to 707 of SEQ ID NO: 14.

50. The cannabis cell of claim 49, wherein the genetic modification is targeted to:
a) a region of at least one CBDAS gene comprising a nucleotide sequence corresponding to nucleotides 98 to 344 of SEQ ID NOs: 18 or 19, nucleotides 134 to 380 of SEQ ID NO: 20, nucleotides 272 to 518 of SEQ ID NOs: 21 or 22, nucleotides 236 to 482 of SEQ ID NO: 24, nucleotides 1 to 35 of SEQ ID NO: 25, or nucleotides 158 to 404 of SEQ ID NO: 26;
b) a region of at least one CBCAS gene comprising a nucleotide sequence corresponding to nucleotides 272 to 518 of SEQ ID NO: 15, 16 or 17; and
c) a region of a THCAS gene comprising a nucleotide sequence corresponding to nucleotides 272 to 518 of SEQ ID NO: 14.

51. The cannabis cell of claim 48, wherein one or more, or all of the following applies:
i) the genetic modification is targeted to a nucleotide sequence that is shared between the regions of (a)-(c);
ii) the genetic modification is targeted to a region of at least one CBDAS gene of (a);
iii) the genetic modification is targeted to a region of at least one CBCAS gene of (b);
iv) the genetic modification is targeted to a region of a THCAS gene of (c); and
v) the genetic modification produces the altered expression of the cannabinoid biosynthesis genes via gene silencing.

52. The cannabis cell of claim 51, wherein the gene silencing is mediated by a small interfering RNA (siRNA).

53. The cannabis cell of claim 52, wherein one of the following applies:
i) the siRNA targets THCAS and is selected from SEQ ID NOs: 27-119, or a nucleotide sequence which is at least 90% identical to the nucleotide sequence of SEQ ID NOs: 27-119;
ii) the siRNA targets a CBDAS gene and is selected from SEQ ID NOs: 120-189 and 285-322, or a nucleotide sequence which is at least 90% identical to the nucleotide sequence of SEQ ID NOs: 120-189 and 285-322; and
iii) the siRNA targets a CBCAS gene and is selected from SEQ ID NOs: 190-284, or a nucleotide sequence which is at least 90% identical to the nucleotide sequence of SEQ ID NOs: 190-284.

54. The cannabis cell of claim 48, which is derived from a Cannabis spp. selected from the group consisting of Cannabis sativa, Cannabis indica and Cannabis ruderalis.

55. The cannabis cell of claim 48, wherein one of the following applies:
i) the genetic modification produces a transient alteration of the expression of the cannabinoid biosynthesis genes; and
ii) the genetic modification produces a transient reduction in the expression of the cannabinoid biosynthesis genes.

56. The cannabis cell of claim 55, wherein one of the following applies:
i) THCAS expression is reduced by at least about 30% relative to a cannabis cell that does not comprise the genetic modification;
ii) THCAS expression is reduced by from about 30% to about 100% relative to a cannabis cell that does not comprise the genetic modification;
iii) CBDAS expression is reduced by at least about 50% relative to a cannabis cell that does not comprise the genetic modification;
iv) CBDAS expression is reduced by from about 50% to about 100% relative to a cannabis cell that does not comprise the genetic modification;
v) CBCAS expression is reduced by at least about 25% relative to a cannabis cell that does not comprise the genetic modification; and
vi) CBCAS expression is reduced by from about 25% to about 90% relative to a cannabis cell that does not comprise the genetic modification.

57. The cannabis cell of claim 48, which is a regenerable cell.

58. A cannabis plant comprising the cell of claim 48.

59. A plant extract obtained from the cannabis plant of claim 58.

60. A synthetic siRNA molecule comprising a nucleotide sequence selected from SEQ ID NOs: 27-322, or a nucleotide sequence which is at least 90% identical to the nucleotide sequence of SEQ ID NOs: 27-322.

61. A vector comprising a nucleic acid molecule encoding the siRNA molecule of claim 60.

62. A method for producing a cannabis plant with an altered level or activity, or both, of a cannabinoid biosynthesis pathway, the method comprising:
a) introducing a targeted genetic modification to a cannabis cell leading to altered expression of one or more cannabinoid biosynthesis genes, wherein the genetic modification is targeted to one or more or all of:
i) a region of at least one CBDAS gene comprising a nucleotide sequence corresponding to any one of SEQ ID NOs: 18-26, or a or a nucleotide sequence which is at least 90% identical to the nucleotide sequence of SEQ ID NOs: 18-26;
ii) a region of at least one CBCAS gene comprising a nucleotide sequence corresponding to any one of SEQ ID NOs: 15-17, or a or a nucleotide sequence which is at least 90% identical to the nucleotide sequence of SEQ ID NOs: 15-17; and
iii) a region of a THCAS gene comprising a nucleotide sequence corresponding to SEQ ID NO: 14, or a or a nucleotide sequence which is at least 90% identical to the nucleotide sequence of SEQ ID NO: 14; and
b) regenerating a cannabis plant from the cell of step (a), wherein the plant comprises the genetic modification and has altered level or activity, or both, of the cannabinoid biosynthesis pathway relative to a cannabis plant that does not comprise the genetic modification.

63. The method of claim 62, wherein the genetic modification produces the altered level or activity, or both, of the cannabinoid biosynthesis pathway via gene silencing.

64. The method of claim 62, further comprising one or both of the following steps:
i) crossing the plant with the genetic modification with a second plant to produce one or more progeny plants; and
ii) harvesting plant material from the plant.

65. The method of claim 64, wherein the plant material comprises cannabis inflorescences.

66. A transgenic cannabis plant comprising a RNA interference (RNAi) vector comprising a nucleotide sequence encoding a siRNA, wherein the siRNA targets one or more or all of a THCAS gene, at least one CBDAS gene and at least one CBCAS gene.

67. The transgenic cannabis plant of claim 66, wherein the siRNA comprises a nucleotide sequence selected from SEQ ID NOs: 27-322, or a nucleotide sequence which is at least 90% identical to the nucleotide sequence of SEQ ID NOs: 27-322.


SEQUENCE LISTING (346 sequences)

SEQ ID NO: 1 - DNA, 1868 bp, Cannabis sativa
  source 1..1868: mol_type = other DNA
aaaaaatcattaggactgaagaaaaatgaattgctcagcattttccttttggtttgtttgcaaaataatatttttctttctctcattccatatccaaatttcaatagctaatcctcgagaaaacttccttaaatgcttctcaaaacatattcccaacaatgtagcaaatccaaaactcgtatacactcaacacgaccaattgtatatgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcctgagttgggtattaaaaaaactgattgcaaagaatttagctggattgatacaaccatcttctacagtggtgttgtaaattttaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcattaattatctttaaatagatatatttcccttatcaattagttaatcattataccatacatacatttattgtatatagtttatctactcatattatgtatgctcccaagtatgaaaatctacattagaactgtgtagacaatcataagatatatttaataaaataaattgtctttcttatttcaatagcaaataaaataatattatttta

SEQ ID NO: 2 - DNA, 1635 bp, Cannabis sativa
  source 1..1635: mol_type = other DNA
atgaattgctcaacattctccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcgtcatcat

SEQ ID NO: 3 - DNA, 1635 bp, Cannabis sativa
  source 1..1635: mol_type = other DNA
atgaattgctcaacattctccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaattcatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcgtcatcat

SEQ ID NO: 4 - DNA, 1157 bp, Cannabis sativa
  source 1..1157: mol_type = other DNA
atgaattgctcaacattctccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatccatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttaga

SEQ ID NO: 5 - DNA, 1633 bp, Cannabis sativa
  source 1..1633: mol_type = other DNA
tgatgacgtaggggaagaggtgggatgctttgttcgtttctaaagaaattatcgggatcaaccttggtttttacttttactaacctattaaaatttttaccaaaatacttttcaccccaaatacgtgcttgtgtgtaattattaggactctcgaaattagtttttcctaaatcaaggtccctataattgagatacgccattcttggattttgggacacataaggagtcgtgaaattataaacattccgaatccagtttatatgcttttcattatcttcttgcttctcccatgaagctatgtaccaaatttcatacatgattccagctcgatgagggaatggaattgctgattctgaaatctcatccattataccaccataagggtaaaacacaaacatcccaactcctacatcttcttcatataatttttccaaaattgtgaccattgcggtttctggaatcggtttcttaacatagtctaacttaatcgagaaagccgccttccgcccacctgatctatcaagcaaaatttcttttttaaaattagttgtgttgtaatttacaacaccactgtagaagatgatagtatcaatccagctcaactgtttgcaatctgtttttttaatacccaattcaggaaagctcttgttcatcaagtcgactagactatccactccaccatggaaaatggaggagaagtaactgtgtattgttgtcttattcttcccttgattatctgtaatattcctggttataaagtgagtaaagagtaataattctttttcatacatgtaagcaatattttgccatttgttaactaacttgacaagctcatgtatctccatgttctttttaacactgaatatagtagacattgatgggacagcaacaagtctaattttccacgctgcaatgattccaaagttttctcctccaccaccacgtatagcccaaaacaaatcttcccccatggattttcgatctaaaacttttccatcaacattgactaagtgcgcatcaatgatattatcagccgcgaggccataatttcgcatcaatgctccatagcctcctccactaaagtgtccacccgcgccaacagtagggcagtacccagcaggaaaactaagattctcattgttctcattgatccaataataaacttctccaagggtagctccggcttcaacccatgcagtttggctatgaacatctattttgaccgaatgcatgtttctcaagtctactataacaaatgggacttcagatatgtaggacatgccctcagcatcatgaccaccgcttcgagttcgaatctgcaagccaactttcttggagcatagaatagtgccttggatatgggagacatttaaaggagtgatgataacaagtggttttggggttgtttcagaggtaaatctaagattttgtatggtcgaatttaggatagacatataaaattggtcgtgttgagtgtatacgagttttgcatttgttacattggtgggaatatattgtgagaagcatttaaggaagttttcttgaggattagctattgaaatttggatattgaatgagagaaagaaaaatattatcttgcaaacataccaaaaacagaatgttgagtacttca

SEQ ID NO: 6 - DNA, 1633 bp, Cannabis sativa; identical to SEQ ID NO: 5
  source 1..1633: mol_type = other DNA

SEQ ID NO: 7 - DNA, 1609 bp, Cannabis sativa
  source 1..1609: mol_type = other DNA
tgatgacgcaggggaagaggtgggatgctttgttcgtttctaaagaaattatcgggatcaaccttggtttttacttttactaacctattaaaatttttaccaaaatacttttcaccccaaatacgtgcttgtgtgtaattattaggtctctcaaaattagtttttcctaaatcaaggtccctataattgagatacgccattcttggattttgggacacataaggagtcgtgaaattataaacattccgaatccagtttatatgcttttcattatcttcttgcttctcccatgaagctatgtaccaaatttcatacatgattccagctcgatgagggaatggaattgctgattctgaaatctcatccattataccaccataagggtaaaacacaaacatcccaactcctacatcttcttcatataatttttccaaaattgtgaccattgcggtttctggaatcggtttcttaacatagtctaacttaatcgagaaagccgccttccgcccacctgatctatcaagcaaaatttcttttttaaaattagttgtgttgtaatttacaagaccactgtagaagatgatagtatcaatccagctcaactgtttgcaatctatttttttaatacccaattcaggaaagctcttgttcatcaagtcgactagactatccactccaccatggaaaatggaggagaagtaactgtgtattgttgtcttattcttcccttgattatctgtaatattcctggttataaagtgagtaaagagtaataattctttttcatacatgtaagcaatattttgccatttgttaactaacttgacaagctcatgtatctccatgttctttttaacactgaatatagtagacattgatgggacagcaacaagtctaattttccacgctacaatgattccaaagttttctcctccaccaccacgtatagcccaaaacaaatcttcccccatggattttcgatctaaaacttttccatcaacattgactaagtgcgcatcaatgatattatcagccgcgaggccataatttcgcatcaatgctccatagcctcctccactaaagtgtccacccgcgccaacagtagggcagtacccagcaggaaaactaagattctcattgttctcattgatccaataataaacttctccaagggtagctccggcttcaacccatgcagtttggctatgaacatctattttgaccgaatgcatgtttctcaagtctactataacaaatgggacttgagatatgtaggacatgccctcagcatcatgaccaccgcttcgagttcgaatctgcaagccaacttttttggagcatagaatagtgccttggatatgggagacatttaaaggagtgatgataacaagtggttttggggttgtgtcagaggtaaatctaagattttgtatggtcgaatttaggatagacatataaaattggtcgtgttgagtgtatacgagttttgcatttgttacattggtgggaatatattgtgagaagcatttaaggaagttttcttgaggattagctattgaaatttggatattgaatgagagaaaaaaaaatattatcttgcaaacatacc

SEQ ID NO: 8 - DNA, 1633 bp, Cannabis sativa
  source 1..1633: mol_type = other DNA
tgatgacgcaggggaagaggtgggatgctttgttcgtttctaaagaaattatcgggatcaaccttggtttttacttttactaacctattaaaatttttaccaaaatacttttcaccccaaatacgtgcttgtgtgtaattattaggactctcgaaattagtttttcctaaatcaaggtccctataattgagatacgccattcttggattttgggacacataaggagtcgtgaaattataaacattccgaatccagtttatatgcttttcattatcttcttgcttctcccatgaagctatgtaccaaatttcatacatgattccagctcgatgagggaatggaattgctgattctgaaatctcatccattataccaccataagggtaaaacacaaacatcccaactcctacatcttcttcatataatttttccaaaattgtgaccattgcggtttctggaatcggtttcttaacatagtctaacttaatcgagaaagccgccttccgcccacctgatctatcaagcaaaatttcttttttaaaattagttgtgttgtaatttacaagaccactgtagaagatgatagtatcaatccagctcaactgtttgcaatctctttttttaatacccaattcaggaaagctcttgttcatcaagtcgactagactatccactccaccatggaaaatggaggagaagtaactgtgtattgttgtcttattcttcccttgattatctgtaatattcctggttataaagtgagtaaagagtaataattctttttcatacatgtaagcaatattttgccatttgttaactaacttgacaagctcatgtatctccatgttctttttaacactgaatatagtagacattgatgggacagcaacaagtctaattttccacgctgcaatgattccaaagttttctcctccaccaccacgtatagcccaaaacaaatcttcccccatggattttcgatctaaaacttttccatcaacattgactaagtgtgcatcaatgatattatcagccgcgaggccataatttcgcatcaatgctccatagcctcctccactaaagtgtccaccagcgccaacagtagggcagtacccagcaggaaaactaagattctcattgttctcattgatccaataataaacttctccaagggtagctccggcttcaacccatgcattttggctatgaacatctattttgaccgaatgcatgtttctcaagtctactataacaaatgggacttgagatatgtaggacatgccctcagcatcatgaccaccgcttcgagttcgaatctgcaagccaactttcttggagcatagaatagtgccttggatatgggagacatttaaaggagtgatgataacaagtggttttggggttgtgtcagaggtaaatctaagattttgtatggtcgaatttaagatagacatataaaattggtcgtgttgagtgtatacgagttttgcatttgttacattggtgggaatatattgtgagaggcatttaaggaagttttcttgaggattagctattgaaatttggatattgaatgagagaaagaaaaatattatcttgcaaacataccaaaaacagaatgttgagtacttca

SEQ ID NO: 9 - DNA, 1633 bp, Cannabis sativa
  source 1..1633: mol_type = other DNA
tgatgacgcaggggaagaggtgggatgctttgttcgtttctaaagaaattatcgggatcaaccttggtttttacttttactaacctattaaaatttttaccaaattacttttcaccccaaatacgtgcttgtgtgtaattattaggactctcgaaattagtttttcctaaatcaaggtccctataattgagatacgccattcttggattttgggacacataaggagtcgtgaaattataaacattccgaatccagtttatatgcttttcattatcttcttgcttctcccatgaagctatgtaccaaatttcatacgtgattccagctcgatgagggaatggaattgctgattctgaaatctcatccattataccaccataagggtaaaacacaaacatcccaactcctacatcttcttcatataatttttccaaaattgtgaccattgcggtttctggaatcggtttcttaacatagtctaacttaatcgagaaagccgccttccgcccacctgatctatcaagcaaaatttcttttttaaaattagttgtgttgtaatttacaacaccactgtagaagatgatagtatcaatccagctcaactgtttgcaatctttttttttaatacccaattcaggaaagctcttgttcatcaagtcgactagactatccactccaccatggaaaatggaggagaagtaactgtgtattgttgtcttattcttcccttgattatctgtaatattcctggttataaagtgagtaaagagtaataattctttttcatacatgtaagcaatattttgccatttgttaactaacttgacaagctcatgtatctccatgttctttttaacactgaatatagtagacattgatgggacagcaacaagtctaattttccacgctgcaatgattccaaagttttctcctccaccaccacgtatagcccaaaacaaatcttcccccatggattttcgatctaaaacttttccatcaacattgactaagtgcgcatcaatgatattatcagccgcgaggccataatttcgcatcaatgctccatagcctcctccactaaagtgtccacccgtgccaacagtagggcagtacccagcaggaaaactaagattctcattgttctcattgatccaataataaacttctccaagggtagctccggattcaacccatgcagtttggctatgaacatctattttgaccgaatgcatgtttctcaagtctactataacaaatgggacttgagatatgtaggacatgccctcagcatcatgaccaccgcttcgagttcgaatctgcaagccaactttcttggaacatagaatagtgccttggatatgggagacatttaaaggagtggtgataacaagtggttttggggttgtgtcagaggtaaatctaagattttgtacggtcgaatttaagatagacatataaaattggtcgtgttgagtgtatacgagttttgcatttgttacattggtgggaatatattgtgagaggcatttaaggaagttttcttgaggattagctattgaaatttggatattgaatgagagaaagaaaaatattatcttgcaaacataccaaaaacagaatgttgagtacttca

SEQ ID NO: 10 - DNA, 977 bp, Cannabis sativa
  source 1..977: mol_type = other DNA
tgatgacgtaggggaagaggtgggatgctttgttcgtttctaaagaaattatcgggatcaaccttggtttttacttttactaacctattaaattttttaccaaaatacttttcaccccaaatacgtgcttgtgtgtaattattaggactctcgaaattagtttttcctaaatcaaggtccctataattgagatacgccattcttggattttgggacacataaggagtcgtgaaattgtaaacattctgaatccagtttatatgcttttcattatcttcttgcttctcccatgaagctatgtaccaaatttcatacatgattccagctcgatgagggaatggaattgctgattctgaaatctcatccattattccaccataagggtaaaacacaaacatcccaactcctacatcttcttcatataatttttccaaaattgtgaccattgcggtttctggaatcggtttcttaacttagtctaacttaatcgagaaagccgccttccgcccacctgatctatcaagcaaaagttcttttttaaaattagtagtgttgtaatttacaagaccactgtagaagatgatagtatcaatccagctcaactgtttgcaatctgtttttttaatacccaattcaggaaagctcttgttcatcaagtcgactagactatccactccaccatggaaaatggaggagaagtaactatgtattgttgtcttattctttccttgattatctgtaatattcctggttataaagtgagtaaagagtaataattctttttcatacatgtaagcaatattttgccatttgttaactaacttgacaagctcatgtatctccatgttctttttattactgaatatagtagacattgatgggacagcaacaagtctaattttccacgctgcaatgattccaaagttttctcctccaccaccacgtatagcccaaaacaaatcttcccccatggattt

SEQ ID NO: 11 - DNA, 1712 bp, Cannabis sativa
  source 1..1712: mol_type = other DNA
tatttatgttataaggactaataatttgataagagaaatagatctatttaagataattaatgatgacgcaggggaagaggtgggatgctttgttcgtttctaaagaaattatcgggatcaaccttggtttttacttttactaacctactaaaatttttaccaaaatacttttcaccccaaatacgtgcttgtgtgtaattattaggactctcgaaattagtttttcctaaatcaaggtccctataattgagatacgccattcttggattttgggacacataaggagtcgtgaaattataaacattccgaatccagtttatatgcttttcattatcttcttgcttctcccatgaagctatgtaccaaatttcatacatgattccagctcgatgagggaatggaattgctgattctgaaatctcatccattataccaccataagggtaaaacacgaacatcccaactcctacatcttcttcatataatttttccaaaattgtgaccattgcggtttctggaatcggtttcttaacatagtctaacttaattgagaaagccgccttccgcccacctgatctttaaagcaaaatttcttttttaaaattagtagtgttgtaatttacaagaccactgttgaagatgatagtatcaatccagctcaactgtttgcaatctgtttttttaatacccaattcaggaaagctcttgttcatcaagtcgactagactatccagtccaccatggaaaatggaggagaagtaacagtgtattgttgtcttattcttcccttgattatctgtaatattcctggttataaagtgagtaaagagtaataattctttttcatacatgtaagcaatattttgccattttttaactaacttgacaagctcatgtatctccatgttctttttaacactgaatatagtagacattgatgggacagcaacaagtctaattttccacgctgcaatgattccagagttttctcctccaccaccacgtatagcccaaaacaaatcttcccccatggattttcgatctaaaacttttccatcaacattgactaagtgcgcatcaatgatattatcagccgcgaggccataatttcgcatcaatgctccatagcctcctccactaaagtgtccacccgcgccaacagtaggacagtacccagcaggaaaactaagattctcattgttctcattgatccaataataaacttctccaagggtagctccggcttcaacccatgcagtttggctatgaacatctattttgaccgaatgcatgtttctcaagtctactataacaaatgggacttgagatatgtaggacatgccctcagcatcatgaccactgcttcgagctcgaatctgcaagccaactttcttggagcatagaatagtgccttggatatgggagacatttaaaggagcgatgataacaagtggttttggggttgtgtcagaggtaaatctaagattttgtatggtcgaatttaggatagacatataaaattggtcgtgttgagtgtatacgagttttgcatttgttacattggtgggaatatattgtgagaagcatttaaggaagttttcttgaggattagctattgaaatttggatattgaatgagagaaagaaaaatattatcttgcaaacataccaaaaacagaatgttgagtacttcatttttctttagtcgtaatg

SEQ ID NO: 12 - DNA, 1652 bp, Cannabis sativa
  source 1..1652: mol_type = other DNA
tgacgatgcggtggaagaggtgggatgctttgttcatttctaaaaagattattgaaatcaaccagggttttcacttttactagcctgtcaaaatttttaccaaagatttctcacccaaaatacatgcttctgtgtaattattttgactcttgggatcatttattcctatatcaagatgtctataattgagatatgtcaatcttggattttgggacacataaggagtcatgaagttatcaacatttcgaatccagtttagatacttttctttatcttcgtgcttctcccagctacatatgtaccataattcatacatgattccaactcgatgagggaatggaattgctgattctgaaatctcatccattagacaaccgtaagggtacaacgcatacatcccaactcctttatcttcttcatataatttttccaaaattttgacaaatgcagattctggaattggtttcttaacatagtctaacttaatcttgaaagcaccgttctgcccagctgatctaacaagcgaaatttggttattaaaattatcagtgccgtaatttacaacaccgctatagaagatgatagtatcaatccagctcaattgtttgcaatctatttttttaataccaaactcaggaaaactcttgttcattaagtcgactagactatcgactccaccaaggaaaactaaagagaagtaagtgtgtattgttgtcttattcttcccttgattatctgtaatattcctagttatgaagtgagtcatgagtaataaatctatgtcatacttgtaagaaatattttgccacttgttaactaacttcacaagctcatgtatctccttgatctttttaacactaaacatagtagactttgttgggacagcaaccagtctaattttccatgctacaatgattctgaagctttctcttccaccaccacgtatagcccaaaagagatcttgtcccatagattttcgatctagtacttttccatcaacgttgactaagtgtgcatcaacgatattatcatccgcgaggccataatttcacatcaatggtccatagcctcctccaccaaagtgtccagctgcgcaaatagtagggcaatacccagcacccaaactaagattctcatttttctcattaacccaataataaacttctccaatggtagctccggcttcaacccatgtagtttggctatgaacatctattttgattgaatgcatgtttctcaagtctactataacaaatgggacttgagatatgtgggacatgccttcagaatcatgaccaccgtttcgagtttgaatttgcaaaccaattttcttggacatagaatagtgccttggatatgggagacatgtgaaggagtgacgataacaagtggtttttgggttgtgttagaggtgaatctaagattgtgtattgtcgaattttggacaggcatatacaattggttgttttgagtgtaagttttagatttgttacattggtgggaatatattgcgagaagcatttaaggaagttttctcgaggattagcaattgaaggttggatattgaatgagagaaagaaaaatattatcttgcaaacaaaccaaaaggagaatgttgagtacttcatttttctttagttctaataattttt

SEQ ID NO: 13 - DNA, 1380 bp, Cannabis sativa
  source 1..1380: mol_type = other DNA
tgacgatgcggtggaagaggtgggatgctttgttcatttctaaaaaaattattgggatcaaccagggttttcacttttactaccctgtcaaaatttttaccaaaatacttctcaccccaaatacttgcttgtgtgtaattatttggactcttgggatcatttattccagtatcaaggtctctataattgagatatgccaatcttggattttaggacacataaggagtaatgaagctataaacatttcgaatccagtttagatgctttttcgttatcttcgtgcttctcccagctacatatgtaccataattcatacatgattccagctcaatgaggtaatggaattgctgattctgaaatctcatccattataccaccgtaagggtacaacgcatacatcccagctccttcatcttcttcatataatttttccaaaattttgacaaacgcagattctggaattggtttcttaacgtagtctaacttaatctttaaagaaccgttctgcccagctgatctatcaagcaaaatttccttattaaaattatcagtgccgtaatttacaacaccgctataaaagatgataatatcaatctagctcaattgtttgcaatctgtttttttaatacccaactcaggaaaactcttattcatcaagtcgactagactatccactccaccaaggaaaactgaagagaagtaagtgtgtattgttgtcttattcttcccatgattatttgtaatattcctagttatgaagtgagtcatgagtaataaatctttgtcatacttgtaagcaatattttgccatttgttaactcacttgacaagctcatgtatctccatgatctttttaacactaaacatagtagactttgttgggacagcaaccagtctaattttccatgctacaatgattccgaagctttctcctccaccaccacgtatagcccaaaagagatcttcccccatagattttcgatctagcacttttgcatcaacgttgactaagtgtgcatcaacgatattatcagccgcgaggccataattttgcatcaatggtccatatcctcctccaccaaagtgtccagctgcgctaacagtagggcaatacccagcagccaaactaagattctcatttttctcattaacccaataataaacttctccaagggtagctccggcttcaacccttgcgatttggctatgaacatctatgttgattgaatgcatgtttctcaagtctactataacaaatgggacttgagatatgtaggacatgtcttcagaatcatgaccaccgcttcgagttcgaatttgcaaaccaattttcttggacatagaatagtgccttggatatgagagacatgtgaaggagtg

SEQ ID NO: 14 - DNA, 1458 bp, Cannabis sativa
  source 1..1458: mol_type = other DNA
atgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcctgagttgggtattaaaaaaactgattgcaaagaatttagctggattgatacaaccatcttctacagtggtgttgtaaattttaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcattaa

SEQ ID NO: 15 - DNA, 1380 bp, Cannabis sativa
  source 1..1380: mol_type = other DNA
atgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaag

SEQ ID NO: 16 - DNA, 1380 bp, Cannabis sativa
  source 1..1380: mol_type = other DNA
atgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaattcatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaag

SEQ ID NO: 17 - DNA, 969 bp, Cannabis sativa
  source 1..969: mol_type = other DNA
atgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatccatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaatt

SEQ ID NO: 18 - DNA, 1218 bp, Cannabis sativa
  source 1..1218: mol_type = other DNA
atgtcctacatatctgaagtcccatttgttatagtagacttgagaaacatgcattcggtcaaaatagatgttcatagccaaactgcatgggttgaagccggagctacccttggagaagtttattattggatcaatgagaacaatgagaatcttagttttcctgctgggtactgccctactgttggcgcgggtggacactttagtggaggaggctatggagcattgatgcgaaattatggcctcgcggctgataatatcattgatgcgcacttagtcaatgttgatggaaaagttttagatcgaaaatccatgggggaagatttgttttgggctatacgtggtggtggaggagaaaactttggaatcattgcagcgtggaaaattagacttgttgctgtcccatcaatgtctactatattcagtgttaaaaagaacatggagatacatgagcttgtcaagttagttaacaaatggcaaaatattgcttacatgtatgaaaaagaattattactctttactcactttataaccaggaatattacagataatcaagggaagaataagacaacaatacacagttacttctcctccattttccatggtggagtggatagtctagtcgacttgatgaacaagagctttcctgaattgggtattaaaaaaacagattgcaaacagttgagctggattgatactatcatcttctacagtggtgttgtaaattacaacacaactaattttaaaaaagaaattttgcttgatagatcaggtgggcggaaggcggctttctcgattaagttagactatgttaagaaaccgattccagaaaccgcaatggtcacaattttggaaaaattatatgaagaagatgtaggagttgggatgtttgtgttttacccttatggtggtataatggatgagatttcagaatcagcaattccattccctcatcgagctggaatcatgtatgaaatttggtacatagcttcatgggagaagcaagaagataatgaaaagcatataaactggattcggaatgtttataatttcacgactccttatgtgtcccaaaatccaagaatggcgtatctcaattatagggaccttgatttaggaaaaactaatttcgagagtcctaataattacacacaagcacgtatttggggtgaaaagtattttggtaaaaattttaataggttagtaaaagtaaaaaccaag

SEQ ID NO: 19 - DNA, 1218 bp, Cannabis sativa; identical to SEQ ID NO: 18
  source 1..1218: mol_type = other DNA

SEQ ID NO: 20 - DNA, 1254 bp, Cannabis sativa
  source 1..1254: mol_type = other DNA
attcgaactcgaagcggtggtcatgatgctgagggcatgtcctacatatctcaagtcccatttgttatagtagacttgagaaacatgcattcggtcaaaatagatgttcatagccaaactgcatgggttgaagccggagctacccttggagaagtttattattggatcaatgagaacaatgagaatcttagttttcctgctgggtactgccctactgttggcgcgggtggacactttagtggaggaggctatggagcattgatgcgaaattatggcctcgcggctgataatatcattgatgcgcacttagtcaatgttgatggaaaagttttagatcgaaaatccatgggggaagatttgttttgggctatacgtggtggtggaggagaaaactttggaatcattgtagcgtggaaaattagacttgttgctgtcccatcaatgtctactatattcagtgttaaaaagaacatggagatacatgagcttgtcaagttagttaacaaatggcaaaatattgcttacatgtatgaaaaagaattattactctttactcactttataaccaggaatattacagataatcaagggaagaataagacaacaatacacagttacttctcctccattttccatggtggagtggatagtctagtcgacttgatgaacaagagctttcctgaattgggtattaaaaaaatagattgcaaacagttgagctggattgatactatcatcttctacagtggtcttgtaaattacaacacaactaattttaaaaaagaaattttgcttgatagatcaggtgggcggaaggcggctttctcgattaagttagactatgttaagaaaccgattccagaaaccgcaatggtcacaattttggaaaaattatatgaagaagatgtaggagttgggatgtttgtgttttacccttatggtggtataatggatgagatttcagaatcagcaattccattccctcatcgagctggaatcatgtatgaaatttggtacatagcttcatgggagaagcaagaagataatgaaaagcatataaactggattcggaatgtttataatttcacgactccttatgtgtcccaaaatccaagaatggcgtatctcaattatagggaccttgatttaggaaaaactaattttgagagacctaataattacacacaagcacgtatttggggtgaaaagtattttggtaaaaattttaataggttagtaaaagtaaaaaccaag

SEQ ID NO: 21 - DNA, 1392 bp, Cannabis sativa
  source 1..1392: mol_type = other DNA
atgtctatcttaaattcgaccatacaaaatcttagatttacctctgacacaaccccaaaaccacttgttatcatcactcctttaaatgtctcccatatccaaggcactattctatgctccaagaaagttggcttgcagattcgaactcgaagcggtggtcatgatgctgagggcatgtcctacatatctcaagtcccatttgttatagtagacttgagaaacatgcattcggtcaaaatagatgttcatagccaaaatgcatgggttgaagccggagctacccttggagaagtttattattggatcaatgagaacaatgagaatcttagttttcctgctgggtactgccctactgttggcgctggtggacactttagtggaggaggctatggagcattgatgcgaaattatggcctcgcggctgataatatcattgatgcacacttagtcaatgttgatggaaaagttttagatcgaaaatccatgggggaagatttgttttgggctatacgtggtggtggaggagaaaactttggaatcattgcagcgtggaaaattagacttgttgctgtcccatcaatgtctactatattcagtgttaaaaagaacatggagatacatgagcttgtcaagttagttaacaaatggcaaaatattgcttacatgtatgaaaaagaattattactctttactcactttataaccaggaatattacagataatcaagggaagaataagacaacaatacacagttacttctcctccattttccatggtggagtggatagtctagtcgacttgatgaacaagagctttcctgaattgggtattaaaaaaagagattgcaaacagttgagctggattgatactatcatcttctacagtggtcttgtaaattacaacacaactaattttaaaaaagaaattttgcttgatagatcaggtgggcggaaggcggctttctcgattaagttagactatgttaagaaaccgattccagaaaccgcaatggtcacaattttggaaaaattatatgaagaagatgtaggagttgggatgtttgtgttttacccttatggtggtataatggatgagatttcagaatcagcaattccattccctcatcgagctggaatcatgtatgaaatttggtacatagcttcatgggagaagcaagaagataatgaaaagcatataaactggattcggaatgtttataatttcacgactccttatgtgtcccaaaatccaagaatggcgtatctcaattatagggaccttgatttaggaaaaactaatttcgagagtcctaataattacacacaagcacgtatttggggtgaaaagtattttggtaaaaattttaataggttagtaaaagtaaaaaccaag

SEQ ID NO: 22 - DNA, 1350 bp, Cannabis sativa
  source 1..1350: mol_type = other DNA
atgtctatcttaaattcgaccgtacaaaatcttagatttacctctgacacaaccccaaaaccacttgttatcaccactcctttaaatgtctcccatatccaaggcactattctatgttccaagaaagttggcttgcagattcgaactcgaagcggtggtcatgatgctgagggcatgtcctacatatctcaagtcccatttgttatagtagacttgagaaacatgcattcggtcaaaatagatgttcatagccaaactgcatgggttgaatccggagctacccttggagaagtttattattggatcaatgagaacaatgagaatcttagttttcctgctgggtactgccctactgttggcacgggtggacactttagtggaggaggctatggagcattgatgcgaaattatggcctcgcggctgataatatcattgatgcgcacttagtcaatgttgatggaaaagttttagatcgaaaatccatgggggaagatttgttttgggctatacgtggtggtggaggagaaaactttggaatcattgcagcgtggaaaattagacttgttgctgtcccatcaatgtctactatattcagtgttaaaaagaacatggagatacatgagcttgtcaagttagttaacaaatggcaaaatattgcttacatgtatgaaaaagaattattactctttactcactttataaccaggaatattacagataatcaagggaagaataagacaacaatacacagttacttctcctccattttccatggtggagtggatagtctagtcgacttgatgaacaagagctttcctgaattgggtattaaaaaaaaagattgcaaacagttgagctggattgatactatcatcttctacagtggtgttgtaaattacaacacaactaattttaaaaaagaaattttgcttgatagatcaggtgggcggaaggcggctttctcgattaagttagactatgttaagaaaccgattccagaaaccgcaatggtcacaattttggaaaaattatatgaagaagatgtaggagttgggatgtttgtgttttacccttatggtggtataatggatgagatttcagaatcagcaattccattccctcatcgagctggaatcacgtatgaaatttggtacatagcttcatgggagaagcaagaagataatgaaaagcatataaactggattcggaatgtttataatttcacgactccttatgtgtcccaaaatccaagaatggcgtatctcaattatagggaccttgatttaggaaaaactaatttcgagagtcctaataattacacacaagcacgtatttggggtgaaaagtaa

SEQ ID NO: 23 - DNA, 384 bp, Cannabis sativa
  source 1..384: mol_type = other DNA
atggtcacaattttggaaaaattatatgaagaagatgtaggagttgggatgtttgtgttttacccttatggtggaataatggatgagatttcagaatcagcaattccattccctcatcgagctggaatcatgtatgaaatttggtacatagcttcatgggagaagcaagaagataatgaaaagcatataaactggattcagaatgtttacaatttcacgactccttatgtgtcccaaaatccaagaatggcgtatctcaattatagggaccttgatttaggaaaaactaatttcgagagtcctaataattacacacaagcacgtatttggggtgaaaagtattttggtaaaaaatttaataggttagtaaaagtaaaaaccaag

SEQ ID NO: 24 - DNA, 1116 bp, Cannabis sativa
  source 1..1116: mol_type = other DNA
tttacctctgacacaaccccaaaaccacttgttatcatcgctcctttaaatgtctcccatatccaaggcactattctatgctccaagaaagttggcttgcagattcgagctcgaagcagtggtcatgatgctgagggcatgtcctacatatctcaagtcccatttgttatagtagacttgagaaacatgcattcggtcaaaatagatgttcatagccaaactgcatgggttgaagccggagctacccttggagaagtttattattggatcaatgagaacaatgagaatcttagttttcctgctgggtactgtcctactgttggcgcgggtggacactttagtggaggaggctatggagcattgatgcgaaattatggcctcgcggctgataatatcattgatgcgcacttagtcaatgttgatggaaaagttttagatcgaaaatccatgggggaagatttgttttgggctatacgtggtggtggaggagaaaactctggaatcattgcagcgtggaaaattagacttgttgctgtcccatcaatgtctactatattcagtgttaaaaagaacatggagatacatgagcttgtcaaatcaggtgggcggaaggcggctttctcaattaagttagactatgttaagaaaccgattccagaaaccgcaatggtcacaattttggaaaaattatatgaagaagatgtaggagttgggatgttcgtgttttacccttatggtggtataatggatgagatttcagaatcagcaattccattccctcatcgagctggaatcatgtatgaaatttggtacatagcttcatgggagaagcaagaagataatgaaaagcatataaactggattcggaatgtttataatttcacgactccttatgtgtcccaaaatccaagaatggcgtatctcaattatagggaccttgatttaggaaaaactaatttcgagagtcctaataattacacacaagcacgtatttggggtgaaaagtattttggtaaaaattttagtaggttagtaaaagtaaaaaccaaggttgatcccgataatttctttagaaacgaacaaagcatcccacctcttcccctgcgtcatcattaa

SEQ ID NO: 25 - DNA, 891 bp, Cannabis sativa
  source 1..891: mol_type = other DNA
atgggacaagatctcttttgggctatacgtggtggtggaagagaaagcttcagaatcattgtagcatggaaaattagactggttgctgtcccaacaaagtctactatgtttagtgttaaaaagatcaaggagatacatgagcttgtgaagttagttaacaagtggcaaaatatttcttacaagtatgacatagatttattactcatgactcacttcataactaggaatattacagataatcaagggaagaataagacaacaatacacacttacttctctttagttttccttggtggagtcgatagtctagtcgacttaatgaacaagagttttcctgagtttggtattaaaaaaatagattgcaaacaattgagctggattgatactatcatcttctatagcggtgttgtaaattacggcactgataattttaataaccaaatttcgcttgttagatcagctgggcagaacggtgctttcaagattaagttagactatgttaagaaaccaattccagaatctgcatttgtcaaaattttggaaaaattatatgaagaagataaaggagttgggatgtatgcgttgtacccttacggttgtctaatggatgagatttcagaatcagcaattccattccctcatcgagttggaatcatgtatgaattatggtacatatgtagctgggagaagcacgaagataaagaaaagtatctaaactggattcgaaatgttgataacttcatgactccttatgtgtcccaaaatccaagattgacatatctcaattatagacatcttgatataggaataaatgatcccaagagtcaaaataattacacagaagcatgtattttgggtgagaaatctttggtaaaaattttgacaggctag

SEQ ID NO: 26 - DNA, 762 bp, Cannabis sativa
  source 1..762: mol_type = other DNA
atgtccaagaaaattggtttgcaaattcgaactcgaagcggtggtcatgattctgaagacatgtcctacatatctcaagtcccatttgttatagtagacttgagaaacatgcattcaatcaacatagatgttcatagccaaatcgcaagggttgaagccggagctacccttggagaagtttattattgggttaatgagaaaaatgagaatcttagtttggctgctgggtattgccctactgttagcgcagctggacactttggtggaggaggatatggaccattgatgcaaaattatggcctcgcggctgataatatcgttgatgcacacttagtcaacgttgatgcaaaagtgctagatcgaaaatctatgggggaagatctcttttgggctatacgtggtggtggaggagaaagcttcggaatcattgtagcatggaaaattagactggttgctgtcccaacaaagtctactatgtttagtgttaaaaagatcatggagatacatgagcttgtcaacggtgttgtaaattacggcactgataattttaataaggaaattttgcttgatagatcagctgggcagaacggttctttaaagattaagttagactacgttaagaaaccaattccagaatctgcgtttgtcaaaattttggaaaaattatatgaagaagatgaaggagctgggatgtatgcgttgtacccttacggtggtataatggatgagatttcagaatcagcaattccattacctcattga

SEQ ID NO: 27 - RNA, 21 nt, synthetic construct
  source 1..21: mol_type = other RNA
ttaaataacttgacaagccca

SEQ ID NO: 28 - RNA, 21 nt, synthetic construct
  source 1..21: mol_type = other RNA
taaatctttgtcatacttgta

SEQ ID NO: 29 - RNA, 21 nt, synthetic construct
  source 1..21: mol_type = other RNA
taacttgacaagcccatgtat

SEQ ID NO: 30 - RNA, 21 nt, synthetic construct
  source 1..21: mol_type = other RNA
aagtctactacaacaaatggg

SEQ ID NO: 31 - RNA, 21 nt, synthetic construct
  source 1..21: mol_type = other RNA
tcaacattgactaagtgtgca

SEQ ID NO: 32 - RNA, 21 nt, synthetic construct
  source 1..21: mol_type = other RNA
taaagtgtccacctacgccaa

SEQ ID NO: 33 - RNA, 21 nt, synthetic construct
  source 1..21: mol_type = other RNA
tagactttgatgggacagcaa

SEQ ID NO: 34 - RNA, 21 nt, synthetic construct
  source 1..21: mol_type = other RNA
tactacaacaaatgggacttg

SEQ ID NO: 35 - RNA, 21 nt, synthetic construct
  source 1..21: mol_type = other RNA
taagattctcattcttctcat

SEQ ID NO: 36 - RNA, 21 nt, synthetic construct
  source 1..21: mol_type = other RNA
taacactgaatatagtagact

SEQ ID NO: 37 - RNA, 21 nt, synthetic construct
  source 1..21: mol_type = other RNA
ttcgatctagaacttttccat

SEQ ID NO: 38 - RNA, 21 nt, synthetic construct
  source 1..21: mol_type = other RNA
taatttcgcatcaatgctcca

SEQ ID NO: 39 - RNA, 21 nt, synthetic construct
  source 1..21: mol_type = other RNA
tttgatgggacagcaaccagt

SEQ ID NO: 40 - RNA, 21 nt, synthetic construct
  source 1..21: mol_type = other RNA
ttgatgggacagcaaccagtt

SEQ ID NO: 41 - RNA, 21 nt, synthetic construct
  source 1..21: mol_type = other RNA
ttaacactgaatatagtagac

SEQ ID NO: 42 - RNA, 21 nt, synthetic construct
  source 1..21: mol_type = other RNA
ttctcaagtctactacaacaa

SEQ ID NO: 43 - RNA, 21 nt, synthetic construct
  source 1..21: mol_type = other RNA
tattttgatcgaatgcatgtt

SEQ ID NO: 44 - RNA, 21 nt, synthetic construct
  source 1..21: mol_type = other RNA
tgggacagcaaccagtttgat

SEQ ID NO: 45 - RNA, 21 nt, synthetic construct
  source 1..21: mol_type = other RNA
atcaacattgactaagtgtgc

SEQ ID NO: 46 - RNA, 21 nt, synthetic construct
  source 1..21: mol_type = other RNA
atagcctcctccactaaagtg

SEQ ID NO: 47 - RNA, 21 nt, synthetic construct
  source 1..21: mol_type = other RNA
tgatgggacagcaaccagttt

SEQ ID NO: 48 - RNA, 21 nt, synthetic construct
  source 1..21: mol_type = other RNA
ttgatcgaatgcatgtttctc

SEQ ID NO: 49 - RNA, 21 nt, synthetic construct
  source 1..21: mol_type = other RNA
atctattttgatcgaatgcat

SEQ ID NO: 50 - RNA, 21 nt, synthetic construct
  source 1..21: mol_type = other RNA
tgatccaataataaacttctc

SEQ ID NO: 51 - RNA, 21 nt, synthetic construct
  source 1..21: mol_type = other RNA
aatttcgcatcaatgctccat

SEQ ID NO: 52 - RNA, 21 nt, synthetic construct
  source 1..21: mol_type = other RNA
tttcgcatcaatgctccatag

SEQ ID NO: 53 - RNA, 21 nt, synthetic construct
  source 1..21: mol_type = other RNA
aacactgaatatagtagactt

SEQ ID NO: 54 - RNA, 21 nt, synthetic construct
  source 1..21: mol_type = other RNA
ttgccatttgttaaataactt

SEQ ID NO: 55 - RNA, 21 nt, synthetic construct
  source 1..21: mol_type = other RNA
tattatcagccgcaaggccat

SEQ ID NO: 56 - RNA, 21 nt, synthetic construct
  source 1..21: mol_type = other RNA
tgagtcatgagtactaaatct

SEQ ID NO: 57 - RNA, 21 nt, synthetic construct
  source 1..21: mol_type = other RNA
aacagatcttctcccatggat

SEQ ID NO: 58 - RNA, 21 nt, synthetic construct
  source 1..21: mol_type = other RNA
tgacaagcccatgtatctcca

SEQ ID NO: 59 - RNA, 21 nt, synthetic construct
  source 1..21: mol_type = other RNA
tgacaagcccatgtatctcca

SEQ ID NO: 60 - RNA, 21 nt, synthetic construct
  source 1..21: mol_type = other RNA
tcgaatctgcaagccaacttt

SEQ ID NO: 61 - RNA, 21 nt, synthetic construct
  source 1..21: mol_type = other RNA
tagcctcctccactaaagtgt

SEQ ID NO: 62 - RNA, 21 nt, synthetic construct
  source 1..21: mol_type = other RNA
tgcaagccaactttcttagag

SEQ ID NO: 63 - RNA, 21 nt, synthetic construct
  source 1..21: mol_type = other RNA
tagggcaatacccaccaggaa

SEQ ID NO: 64 - RNA, 21 nt, synthetic construct
  source 1..21: mol_type = other RNA
tgccatttgttaaataacttg

SEQ ID NO: 65 - RNA, 21 nt, synthetic construct
  source 1..21: mol_type = other RNA
tcgagttcgaatctgcaagcc

SEQ ID NO: 66 - RNA, 21 nt, synthetic construct
  source 1..21: mol_type = other RNA
tacaacaaatgggacttgaga

SEQ ID NO: 67 - RNA, 21 nt, synthetic construct
  source 1..21: mol_type = other RNA
aaggccataatttcgcatcaa

SEQ ID NO: 68 - RNA, 21 nt, synthetic construct
  source 1..21: mol_type = other RNA
tcatgagtactaaatctttgt

SEQ ID NO: 69 - RNA, 21 nt, synthetic construct
  source 1..21: mol_type = other RNA
tttgatcgaatgcatgtttct

SEQ ID NO: 70 - RNA, 21 nt, synthetic construct
  source 1..21: mol_type = other RNA
ctttgatgggacagcaaccag

SEQ ID NO: 71 - RNA, 21 nt, synthetic construct
  source 1..21: mol_type = other RNA
ttgacaagcccatgtatctcc

SEQ ID NO: 72 - RNA, 21 nt, synthetic construct
  source 1..21: mol_type = other RNA
tgtccacctacgccaacagta

SEQ ID NO: 73 - RNA, 21 nt, synthetic construct
  source 1..21: mol_type = other RNA
atgaagtgagtcatgagtact

SEQ ID NO: 74 - RNA, 21 nt, synthetic construct
  source 1..21: mol_type = other RNA
atcaatgctccatagcctcct

SEQ ID NO: 75 - RNA, 21 nt, synthetic construct
  source 1..21: mol_type = other RNA
tcaacccacgcagtttggcta

SEQ ID NO: 76 - RNA, 21 nt, synthetic construct
  source 1..21: mol_type = other RNA
tgctgcaatgattccaaagtt

SEQ ID NO: 77 - RNA, 21 nt, synthetic construct
  source 1..21: mol_type = other RNA
tatgaacatctattttgatcg

SEQ ID NO: 78 - RNA, 21 nt, synthetic construct
  source 1..21: mol_type = other RNA
agtctactacaacaaatggga

SEQ ID NO: 79 - RNA, 21 nt, synthetic construct
  source 1..21: mol_type = other RNA
ctaaagtgtccacctacgcca

SEQ ID NO: 80 - RNA, 21 nt, synthetic construct
  source 1..21: mol_type = other RNA
gacaagcccatgtatctccat

SEQ ID NO: 81 - RNA, 21 nt, synthetic construct
  source 1..21: mol_type = other RNA
tacttgtaagcaatattttgc

SEQ ID NO: 82 - RNA, 21 nt, synthetic construct
  source 1..21: mol_type = other RNA
tcaatgctccatagcctcctc

SEQ ID NO: 83 - RNA, 21 nt, synthetic construct
  source 1..21: mol_type = other RNA
tgactaagtgtgcatcaataa

SEQ ID NO: 84 - RNA, 21 nt, synthetic construct
  source 1..21: mol_type = other RNA
gagtcatgagtactaaatctt

SEQ ID NO: 85 - RNA, 21 nt, synthetic construct
  source 1..21: mol_type = other RNA
gtatagcccaaaacagatctt

SEQ ID NO: 86 - RNA, 21 nt, synthetic construct
  source 1..21: mol_type = other RNA
tctccaagggtagctccggct

SEQ ID NO: 87 - RNA, 21 nt, synthetic construct
  source 1..21: mol_type = other RNA
tgcatgtttctcaagtctact

SEQ ID NO: 88 - RNA, 21 nt, synthetic construct
  source 1..21: mol_type = other RNA
tgttaaataacttgacaagcc

SEQ ID NO: 89 - RNA, 21 nt, synthetic construct
  source 1..21: mol_type = other RNA
tgatcgaatgcatgtttctca

SEQ ID NO: 90 - RNA, 21 nt, synthetic construct
  source 1..21: mol_type = other RNA
gtcatgagtactaaatctttg

SEQ ID NO: 91 - RNA, 21 nt, synthetic construct
  source 1..21: mol_type = other RNA
gtctactacaacaaatgggac

SEQ ID NO: 92 - RNA, 21 nt, synthetic construct
  source 1..21: mol_type = other RNA
atgcatgtttctcaagtctac

SEQ ID NO: 93 - RNA, 21 nt, synthetic construct
  source 1..21: mol_type = other RNA
ttcaacccacgcagtttggct

SEQ ID NO: 94 - RNA, 21 nt, synthetic construct
  source 1..21: mol_type = other RNA
cagtagggcaatacccaccag

SEQ ID NO: 95 - RNA, 21 nt, synthetic construct
  source 1..21: mol_type = other RNA
tccatgctgcaatgattccaa

SEQ ID NO: 96 - RNA, 21 nt, synthetic construct
  source 1..21: mol_type = other RNA
atggccaccgcttcgagttcg

SEQ ID NO: 97 - RNA, 21 nt, synthetic construct
  source 1..21: mol_type = other RNA
tccggcttcaacccacgcagt

SEQ ID NO: 98 - RNA, 21 nt, synthetic construct
  source 1..21: mol_type = other RNA
tccaagggtagctccggcttc

SEQ ID NO: 99 - RNA, 21 nt, synthetic construct
  source 1..21: mol_type = other RNA
acctacgccaacagtagggca

SEQ ID NO: 100 - RNA, 21 nt, synthetic construct
  source 1..21: mol_type = other RNA
ctacaacaaatgggacttgag

SEQ ID NO: 101 - RNA, 21 nt, synthetic construct
  source 1..21: mol_type = other RNA
tcatggccaccgcttcgagtt

SEQ ID NO: 102 - RNA, 21 nt, synthetic construct
  source 1..21: mol_type = other RNA
tagctccggcttcaacccacg

SEQ ID NO: 103 - RNA, 21 nt, synthetic construct
  source 1..21: mol_type = other RNA
gttaaataacttgacaagccc

SEQ ID NO: 104 - RNA, 21 nt, synthetic construct
  source 1..21: mol_type = other RNA
ggtagctccggcttcaaccca

SEQ ID NO: 105 - RNA, 21 nt, synthetic construct
  source 1..21: mol_type = other RNA
caagccaactttcttagagca

SEQ ID NO: 106 - RNA, 21 nt, synthetic construct
  source 1..21: mol_type = other RNA
tcgcatcaatgctccatagcc

SEQ ID NO: 107 - RNA, 21 nt, synthetic construct
  source 1..21: mol_type = other RNA
aatacccaccaggaaaactaa

SEQ ID NO: 108 - RNA, 21 nt, synthetic construct
  source 1..21: mol_type = other RNA
gatgggacagcaaccagtttg

SEQ ID NO: 109 - RNA, 21 nt, synthetic construct
  source 1..21: mol_type = other RNA
cgcaaggccataatttcgcat

SEQ ID NO: 110 - RNA, 21 nt, synthetic construct
  source 1..21: mol_type = other RNA
atgggacagcaaccagtttga

SEQ ID NO: 111 - RNA, 21 nt, synthetic construct
  source 1..21: mol_type = other RNA
tggccaccgcttcgagttcga

SEQ ID NO: 112 - RNA, 21 nt, synthetic construct
  source 1..21: mol_type = other RNA
tcagccgcaaggccataattt

SEQ ID NO: 113 - RNA, 21 nt, synthetic construct
  source 1..21: mol_type = other RNA
gtagctccggcttcaacccac

SEQ ID NO: 114 - RNA, 21 nt, synthetic construct
  source 1..21: mol_type = other RNA
agcctcctccactaaagtgtc

SEQ ID NO: 115 - RNA, 21 nt, synthetic construct
  source 1..21: mol_type = other RNA
gaatgcatgtttctcaagtct

SEQ ID NO: 116 - RNA, 21 nt, synthetic construct
  source 1..21: mol_type = other RNA
cgagttcgaatctgcaagcca

SEQ ID NO: 117 - RNA, 21 nt, synthetic construct
  source 1..21: mol_type = other RNA
ctccaagggtagctccggctt

SEQ ID NO: 118 - RNA, 21 nt, synthetic construct
  source 1..21: mol_type = other RNA
ttttgccatttgttaaataac

SEQ ID NO: 119 - RNA, 21 nt, synthetic construct
  source 1..21: mol_type = other RNA
cctacgccaacagtagggcaa

SEQ ID NO: 120 - RNA, 21 nt, synthetic construct
  source 1..21: mol_type = other RNA
tcaacattgactaagtgcgca

SEQ ID NO: 121 - RNA, 21 nt, synthetic construct
  source 1..21: mol_type = other RNA
atgaacatctattttgaccga

SEQ ID NO: 122 - RNA, 21 nt, synthetic construct
  source 1..21: mol_type = other RNA
aagtctactataacaaatggg

SEQ ID NO: 123 - RNA, 21 nt, synthetic construct
  source 1..21: mol_type = other RNA
atcaacattgactaagtgcgc

SEQ ID NO: 124 - RNA, 21 nt, synthetic construct
  source 1..21: mol_type = other RNA
aaaactaagattctcattgtt

SEQ ID NO: 125 - RNA, 21 nt, synthetic construct
  source 1..21: mol_type = other RNA
tagacattgatgggacagcaa

SEQ ID NO: 126 - RNA, 21 nt, synthetic construct
  source 1..21: mol_type = other RNA
tagtagacattgatgggacag

SEQ ID NO: 127 - RNA, 21 nt, synthetic construct
  source 1..21: mol_type = other RNA
taacactgaatatagtagaca

SEQ ID NO: 128 - RNA, 21 nt, synthetic construct
  source 1..21: mol_type = other RNA
ttccacgctacaatgattcca

SEQ ID NO: 129 - RNA, 21 nt, synthetic construct
  source 1..21: mol_type = other RNA
ttaacactgaatatagtagac

SEQ ID NO: 130 - RNA, 21 nt, synthetic construct
  source 1..21: mol_type = other RNA
taatttcgcatcaatgctcca

SEQ ID NO: 131 - RNA, 21 nt, synthetic construct
  source 1..21: mol_type = other RNA
tattttgaccgaatgcatgtt

SEQ ID NO: 132 - RNA, 21 nt, synthetic construct
  source 1..21: mol_type = other RNA
ttctcaagtctactataacaa

SEQ ID NO: 133 - RNA, 21 nt, synthetic construct
  source 1..21: mol_type = other RNA
tccactaaagtgtccacccgc

SEQ ID NO: 134 - RNA, 21 nt, synthetic construct
  source 1..21: mol_type = other RNA
tggattttcgatctaaaactt

SEQ ID NO: 135 - RNA, 21 nt, synthetic construct
  source 1..21: mol_type = other RNA
atagcctcctccactaaagtg

SEQ ID NO: 136 - RNA, 21 nt, synthetic construct
  source 1..21: mol_type = other RNA
tcatgtatctccatgttcttt

SEQ ID NO: 137 - RNA, 21 nt, synthetic construct
  source 1..21: mol_type = other RNA
tgatccaataataaacttctc

SEQ ID NO: 138 - RNA, 21 nt, synthetic construct
  source 1..21: mol_type = other RNA
tatgaacatctattttgaccg

SEQ ID NO: 139 - RNA, 21 nt, synthetic construct
  source 1..21: mol_type = other RNA
acaagtctaattttccacgct

SEQ ID NO: 140 - RNA, 21 nt, synthetic construct
  source 1..21: mol_type = other RNA
aatttcgcatcaatgctccat

SEQ ID NO: 141 - RNA, 21 nt, synthetic construct
  source 1..21: mol_type = other RNA
tttcgcatcaatgctccatag

SEQ ID NO: 142 - RNA, 21 nt, synthetic construct
  source 1..21: mol_type = other RNA
aacactgaatatagtagacat

SEQ ID NO: 143 - RNA, 21 nt, synthetic construct
  source 1..21: mol_type = other RNA
ttgaccgaatgcatgtttctc

SEQ ID NO: 144 - RNA, 21 nt, synthetic construct
  source 1..21: mol_type = other RNA
gacaagctcatgtatctccat

SEQ ID NO: 145 - RNA, 21 nt, synthetic construct
  source 1..21: mol_type = other RNA
tagggcagtacccagcaggaa

SEQ ID NO: 146 - RNA, 21 nt, synthetic construct
  source 1..21: mol_type = other RNA
tagcctcctccactaaagtgt

SEQ ID NO: 147 - RNA, 21 nt, synthetic construct
  source 1..21: mol_type = other RNA
tctactataacaaatgggact

SEQ ID NO: 148 - RNA, 21 nt, synthetic construct
  source 1..21: mol_type = other RNA
caagtctaattttccacgcta

SEQ ID NO: 149 - RNA, 21 nt, synthetic construct
  source 1..21: mol_type = other RNA
atattatcagccgcgaggcca

SEQ ID NO: 150 - RNA, 21 nt, synthetic construct
  source 1..21: mol_type = other RNA
atcaatgctccatagcctcct

SEQ ID NO: 151 - RNA, 21 nt, synthetic construct
  source 1..21: mol_type = other RNA
aattttccacgctacaatgat

SEQ ID NO: 152 - RNA, 21 nt, synthetic construct
  source 1..21: mol_type = other RNA
tgactaagtgcgcatcaatga

SEQ ID NO: 153 - RNA, 21 nt, synthetic construct
  source 1..21: mol_type = other RNA
tattatcagccgcgaggccat

SEQ ID NO: 154 - RNA, 21 nt, synthetic construct
  source 1..21: mol_type = other RNA
tcaacccatgcagtttggcta

SEQ ID NO: 155 - RNA, 21 nt, synthetic construct
  source 1..21: mol_type = other RNA
tcaatgctccatagcctcctc

SEQ ID NO: 156 - RNA, 21 nt, synthetic construct
  source 1..21: mol_type = other RNA
agtctactataacaaatggga

SEQ ID NO: 157 - RNA, 21 nt, synthetic construct
  source 1..21: mol_type = other RNA
tttgaccgaatgcatgtttct

SEQ ID NO: 158 - RNA, 21 nt, synthetic construct
  source 1..21: mol_type = other RNA
ctcatgtatctccatgttctt

SEQ ID NO: 159 - RNA, 21 nt, synthetic construct
  source 1..21: mol_type = other RNA
tctccaagggtagctccggct

SEQ ID NO: 160 - RNA, 21 nt, synthetic construct
  source 1..21: mol_type = other RNA
tgcatgtttctcaagtctact

SEQ ID NO: 161 - RNA, 21 nt, synthetic construct
  source 1..21: mol_type = other RNA
tccacgctacaatgattccaa

SEQ ID NO: 162 - RNA, 21 nt, synthetic construct
  source 1..21: mol_type = other RNA
atgcatgtttctcaagtctac

SEQ ID NO: 163 - RNA, 21 nt, synthetic construct
  source 1..21: mol_type = other RNA
agcaacaagtctaattttcca

SEQ ID NO: 164 - RNA, 21 nt, synthetic construct
  source 1..21: mol_type = other RNA
tagctccggcttcaacccatg

SEQ ID NO: 165 - RNA, 21 nt, synthetic construct
  source 1..21: mol_type = other RNA
gtctactataacaaatgggac

SEQ ID NO: 166 - RNA, 21 nt, synthetic construct
  source 1..21: mol_type = other RNA
ttcaacccatgcagtttggct

SEQ ID NO: 167 - RNA, 21 nt, synthetic construct
  source 1..21: mol_type = other RNA
tgaccgaatgcatgtttctca

SEQ ID NO: 168 - RNA, 21 nt, synthetic construct
  source 1..21: mol_type = other RNA
cagtagggcagtacccagcag

SEQ ID NO: 169 - RNA, 21 nt, synthetic construct
  source 1..21: mol_type = other RNA
cactaaagtgtccacccgcgc

SEQ ID NO: 170 - RNA, 21 nt, synthetic construct
  source 1..21: mol_type = other RNA
tccaagggtagctccggcttc

SEQ ID NO: 171 - RNA, 21 nt, synthetic construct
  source 1..21: mol_type = other RNA
tacaatgattccaaagttttc

SEQ ID NO: 172 - RNA, 21 nt, synthetic construct
  source 1..21: mol_type = other RNA
gtagctccggcttcaacccat

SEQ ID NO: 173 - RNA, 21 nt, synthetic construct
  source 1..21: mol_type = other RNA
ttatcagccgcgaggccataa

SEQ ID NO: 174 - RNA, 21 nt, synthetic construct
  source 1..21: mol_type = other RNA
tgcgcatcaatgatattatca

SEQ ID NO: 175 - RNA, 21 nt, synthetic construct
  source 1..21: mol_type = other RNA
gtagacattgatgggacagca

SEQ ID NO: 176 - RNA, 21 nt, synthetic construct
  source 1..21: mol_type = other RNA
actaaagtgtccacccgcgcc

SEQ ID NO: 177 - RNA, 21 nt, synthetic construct
  source 1..21: mol_type = other RNA
ggtagctccggcttcaaccca

SEQ ID NO: 178 - RNA, 21 nt, synthetic construct
  source 1..21: mol_type = other RNA
tcgcatcaatgctccatagcc

SEQ ID NO: 179 - RNA, 21 nt, synthetic construct
  source 1..21: mol_type = other RNA
ttttgaccgaatgcatgtttc

SEQ ID NO: 180 - RNA, 21 nt, synthetic construct
  source 1..21: mol_type = other RNA
gaggccataatttcgcatcaa

SEQ ID NO: 181 - RNA, 21 nt, synthetic construct
  source 1..21: mol_type = other RNA
tttccacgctacaatgattcc

SEQ ID NO: 182 - RNA, 21 nt, synthetic construct
  source 1..21: mol_type = other RNA
acccgcgccaacagtagggca

SEQ ID NO: 183 - RNA, 21 nt, synthetic construct
  source 1..21: mol_type = other RNA
agcctcctccactaaagtgtc

SEQ ID NO: 184 - RNA, 21 nt, synthetic construct
  source 1..21: mol_type = other RNA
tcagccgcgaggccataattt

SEQ ID NO: 185 - RNA, 21 nt, synthetic construct
  source 1..21: mol_type = other RNA
gatattatcagccgcgaggcc

SEQ ID NO: 186 - RNA, 21 nt, synthetic construct
  source 1..21: mol_type = other RNA
gaatgcatgtttctcaagtct

SEQ ID NO: 187 - RNA, 21 nt, synthetic construct
  source 1..21: mol_type = other RNA
acagtagggcagtacccagca

SEQ ID NO: 188 - RNA, 21 nt, synthetic construct
  source 1..21: mol_type = other RNA
ctccaagggtagctccggctt

SEQ ID NO: 189 - RNA, 21 nt, synthetic construct
  source 1..21: mol_type = other RNA
agtacccagcaggaaaactaa

SEQ ID NO: 190 - RNA, 21 nt, synthetic construct
  source 1..21: mol_type = other RNA
taacactgaatatagtagcct

SEQ ID NO: 191 - RNA, 21 nt, synthetic construct
  source 1..21: mol_type = other RNA
taaatctttgtcatacttgta

SEQ ID NO: 192 - RNA, 21 nt, synthetic construct
  source 1..21: mol_type = other RNA
ttaaataacttgacaagccca

SEQ ID NO: 193 - RNA, 21 nt, synthetic construct
  source 1..21: mol_type = other RNA
taacttgacaagcccatgtat

SEQ ID NO: 194 - RNA, 21 nt, synthetic construct
  source 1..21: mol_type = other RNA
tcaacattgactaagtgtgca

SEQ ID NO: 195 - RNA, 21 nt, synthetic construct
  source 1..21: mol_type = other RNA
taaagtgtccacctacgccaa

SEQ ID NO: 196 - RNA, 21 nt, synthetic construct
  source 1..21: mol_type = other RNA
aagtctactatagcaaatggg

SEQ ID NO: 197 - RNA, 21 nt, synthetic construct
  source 1..21: mol_type = other RNA
tactatagcaaatgggacttg

SEQ ID NO: 198 - RNA, 21 nt, synthetic construct
  source 1..21: mol_type = other RNA
tctgaagtgagtcgtgagcat

SEQ ID NO: 199 - RNA, 21 nt, synthetic construct
  source 1..21: mol_type = other RNA
tttctcaagtctactatagca

SEQ ID NO: 200 - RNA, 21 nt, synthetic construct
  source 1..21: mol_type = other RNA
tactttgaccgtatgcatgtt

SEQ ID NO: 201 - RNA, 21 nt, synthetic construct
  source 1..21: mol_type = other RNA
ttcgaatctgcaaaccaactt

SEQ ID NO: 202 - RNA, 21 nt, synthetic construct
  source 1..21: mol_type = other RNA
ttcgatctagaacttttccat

SEQ ID NO: 203 - RNA, 21 nt, synthetic construct
  source 1..21: mol_type = other RNA
taatttcgcatcaatgctcca

SEQ ID NO: 204 - RNA, 21 nt, synthetic construct
  source 1..21: mol_type = other RNA
aacactgaatatagtagcctt

SEQ ID NO: 205 - RNA, 21 nt, synthetic construct
  source 1..21: mol_type = other RNA
tttggctatgaatatctactt

SEQ ID NO: 206 - RNA, 21 nt, synthetic construct
  source 1..21: mol_type = other RNA
ttaacactgaatatagtagcc

SEQ ID NO: 207 - RNA, 21 nt, synthetic construct
  source 1..21: mol_type = other RNA
atctactttgaccgtatgcat

SEQ ID NO: 208 - RNA, 21 nt, synthetic construct
  source 1..21: mol_type = other RNA
tatgaatatctactttgaccg

SEQ ID NO: 209 - RNA, 21 nt, synthetic construct
  source 1..21: mol_type = other RNA
tcaatgatattatcagccgca

SEQ ID NO: 210 - RNA, 21 nt, synthetic construct
  source 1..21: mol_type = other RNA
tagcctttgatgggacaacaa

SEQ ID NO: 211 - RNA, 21 nt, synthetic construct
  source 1..21: mol_type = other RNA
atcaacattgactaagtgtgc

SEQ ID NO: 212 - RNA, 21 nt, synthetic construct
  source 1..21: mol_type = other RNA
ttgaccgtatgcatgtttctc

SEQ ID NO: 213 - RNA, 21 nt, synthetic construct
  source 1..21: mol_type = other RNA
atagcctcctccactaaagtg

SEQ ID NO: 214 - RNA, 21 nt, synthetic construct
  source 1..21: mol_type = other RNA
tttctcctcctccaccacgta

SEQ ID NO: 215 - RNA, 21 nt, synthetic construct
  source 1..21: mol_type = other RNA
ttcttggagcagagaatactg

SEQ ID NO: 216 - RNA, 21 nt, synthetic construct
  source 1..21: mol_type = other RNA
tttaacactgaatatagtagc

SEQ ID NO: 217 - RNA, 21 nt, synthetic construct
  source 1..21: mol_type = other RNA
tgatccaataataaacttctc

SEQ ID NO: 218 - RNA, 21 nt, synthetic construct
  source 1..21: mol_type = other RNA
aatttcgcatcaatgctccat

SEQ ID NO: 219 - RNA, 21 nt, synthetic construct
  source 1..21: mol_type = other RNA
tttcgcatcaatgctccatag

SEQ ID NO: 220 - RNA, 21 nt, synthetic construct
  source 1..21: mol_type = other RNA
tatctactttgaccgtatgca

SEQ ID NO: 221 - RNA, 21 nt, synthetic construct
  source 1..21: mol_type = other RNA
tcgaatctgcaaaccaacttt

SEQ ID NO: 222 - RNA, 21 nt, synthetic construct
  source 1..21: mol_type = other RNA
ttgccatttgttaaataactt

SEQ ID NO: 223 - RNA, 21 nt, synthetic construct
  source 1..21: mol_type = other RNA
tattatcagccgcaaggccat

SEQ ID NO: 224 - RNA, 21 nt, synthetic construct
  source 1..21: mol_type = other RNA
tcctccaccacgtatagccca

SEQ ID NO: 225 - RNA, 21 nt, synthetic construct
  source 1..21: mol_type = other RNA
tgacaagcccatgtatctcca

SEQ ID NO: 226 - RNA, 21 nt, synthetic construct
  source 1..21: mol_type = other RNA
tatagcccaaaatagatcttc

SEQ ID NO: 227 - RNA, 21 nt, synthetic construct
  source 1..21: mol_type = other RNA
tctgcaaaccaactttcttgg

SEQ ID NO: 228 - RNA, 21 nt, synthetic construct
  source 1..21: mol_type = other RNA
tagggcaatacccaccaggaa

SEQ ID NO: 229 - RNA, 21 nt, synthetic construct
  source 1..21: mol_type = other RNA
tagcctcctccactaaagtgt

SEQ ID NO: 230 - RNA, 21 nt, synthetic construct
  source 1..21: mol_type = other RNA
tgccatttgttaaataacttg

SEQ ID NO: 231 - RNA, 21 nt, synthetic construct
  source 1..21: mol_type = other RNA
tagtagcctttgatgggacaa

SEQ ID NO: 232 - RNA, 21 nt, synthetic construct
  source 1..21: mol_type = other RNA
ctgcaaaccaactttcttgga

SEQ ID NO: 233 - RNA, 21 nt, synthetic construct
  source 1..21: mol_type = other RNA
aaggccataatttcgcatcaa

SEQ ID NO: 234 - RNA, 21 nt, synthetic construct
  source 1..21: mol_type = other RNA
gagtcgtgagcattaaatctt

SEQ ID NO: 235 - RNA, 21 nt, synthetic construct
  source 1..21: mol_type = other RNA
ttgacaagcccatgtatctcc

SEQ ID NO: 236 - RNA, 21 nt, synthetic construct
  source 1..21: mol_type = other RNA
atcaatgctccatagcctcct

SEQ ID NO: 237 - RNA, 21 nt, synthetic construct
  source 1..21: mol_type = other RNA
ttttctcctcctccaccacgt

SEQ ID NO: 238 - RNA, 21 nt, synthetic construct
  source 1..21: mol_type = other RNA
tcaacccacgcagtttggcta

SEQ ID NO: 239 - RNA, 21 nt, synthetic construct
  source 1..21: mol_type = other RNA
atagcaaatgggacttgagat

SEQ ID NO: 240 - RNA, 21 nt, synthetic construct
  source 1..21: mol_type = other RNA
tgtccacctacgccaacagta

SEQ ID NO: 241 - RNA, 21 nt, synthetic construct
  source 1..21: mol_type = other RNA
tgaagtgagtcgtgagcatta

SEQ ID NO: 242 - RNA, 21 nt, synthetic construct
  source 1..21: mol_type = other RNA
tgctgcaatgattccaaagtt

SEQ ID NO: 243 - RNA, 21 nt, synthetic construct
  source 1..21: mol_type = other RNA
atatctactttgaccgtatgc

SEQ ID NO: 244 - RNA, 21 nt, synthetic construct
  source 1..21: mol_type = other RNA
aacaacaagtttgattttcca

SEQ ID NO: 245 - RNA, 21 nt, synthetic construct
  source 1..21: mol_type = other RNA
tctactatagcaaatgggact

SEQ ID NO: 246 - RNA, 21 nt, synthetic construct
  source 1..21: mol_type = other RNA
ctaaagtgtccacctacgcca

SEQ ID NO: 247 - RNA, 21 nt, synthetic construct
  source 1..21: mol_type = other RNA
tcaatgctccatagcctcctc

SEQ ID NO: 248 - RNA, 21 nt, synthetic construct
  source 1..21: mol_type = other RNA
gacaagcccatgtatctccat

SEQ ID NO: 249 - RNA, 21 nt, synthetic construct
  source 1..21: mol_type = other RNA
tacttgtaagcaatattttgc

SEQ ID NO: 250 - RNA, 21 nt, synthetic construct
  source 1..21: mol_type = other RNA
agtctactatagcaaatggga

SEQ ID NO: 251 - RNA, 21 nt, synthetic construct
  source 1..21: mol_type = other RNA
tctccaagggtagctccggct

SEQ ID NO: 252 - RNA, 21 nt, synthetic construct
  source 1..21: mol_type = other RNA
tgaccgtatgcatgtttctca

SEQ ID NO: 253 - RNA, 21 nt, synthetic construct
  source 1..21: mol_type = other RNA
tgcatgtttctcaagtctact

SEQ ID NO: 254 - RNA, 21 nt, synthetic construct
  source 1..21: mol_type = other RNA
tgttaaataacttgacaagcc

SEQ ID NO: 255 - RNA, 21 nt, synthetic construct
  source 1..21: mol_type = other RNA
atgcatgtttctcaagtctac

SEQ ID NO: 256 - RNA, 21 nt, synthetic construct
  source 1..21: mol_type = other RNA
cagtagggcaatacccaccag

SEQ ID NO: 257 - RNA, 21 nt, synthetic construct
  source 1..21: mol_type = other RNA
ttcaacccacgcagtttggct

SEQ ID NO: 258 - RNA, 21 nt, synthetic construct
  source 1..21: mol_type = other RNA
caatgatattatcagccgcaa

SEQ ID NO: 259 - RNA, 21 nt, synthetic construct
  source 1..21: mol_type = other RNA
gtatagcccaaaatagatctt

SEQ ID NO: 260 - RNA, 21 nt, synthetic construct
  source 1..21: mol_type = other RNA
tccatgctgcaatgattccaa

SEQ ID NO: 261 - RNA, 21 nt, synthetic construct
  source 1..21: mol_type = other RNA
atggccaccgcttcgagttcg

SEQ ID NO: 262 - RNA, 21 nt, synthetic construct
  source 1..21: mol_type = other RNA
tccggcttcaacccacgcagt

SEQ ID NO: 263 - RNA, 21 nt, synthetic construct
  source 1..21: mol_type = other RNA
tccaagggtagctccggcttc

SEQ ID NO: 264 - RNA, 21 nt, synthetic construct
  source 1..21: mol_type = other RNA
gtctactatagcaaatgggac

SEQ ID NO: 265 - RNA, 21 nt, synthetic construct
  source 1..21: mol_type = other RNA
acctacgccaacagtagggca

SEQ ID NO: 266 - RNA, 21 nt, synthetic construct
  source 1..21: mol_type = other RNA
accaactttcttggagcagag

SEQ ID NO: 267 - RNA, 21 nt, synthetic construct
  source 1..21: mol_type = other RNA
tagctccggcttcaacccacg

SEQ ID NO: 268 - RNA, 21 nt, synthetic construct
  source 1..21: mol_type = other RNA
tcatggccaccgcttcgagtt

SEQ ID NO: 269 - RNA, 21 nt, synthetic construct
  source 1..21: mol_type = other RNA
gttaaataacttgacaagccc

SEQ ID NO: 270 - RNA, 21 nt, synthetic construct
  source 1..21: mol_type = other RNA
atagtagcctttgatgggaca

SEQ ID NO: 271 - RNA, 21 nt, synthetic construct
  source 1..21: mol_type = other RNA
ggtagctccggcttcaaccca

SEQ ID NO: 272 - RNA, 21 nt, synthetic construct
  source 1..21: mol_type = other RNA
aatacccaccaggaaaactaa

SEQ ID NO: 273 - RNA, 21 nt, synthetic construct
  source 1..21: mol_type = other RNA
tcgcatcaatgctccatagcc

SEQ ID NO: 274 - RNA, 21 nt, synthetic construct
  source 1..21: mol_type = other RNA
gtatgcatgtttctcaagtct

SEQ ID NO: 275 - RNA, 21 nt, synthetic construct
  source 1..21: mol_type = other RNA
cgcaaggccataatttcgcat

SEQ ID NO: 276 - RNA, 21 nt, synthetic construct
  source 1..21: mol_type = other RNA
tggccaccgcttcgagttcga

SEQ ID NO: 277 - RNA, 21 nt, synthetic construct
  source 1..21: mol_type = other RNA
agcaaatgggacttgagatat

SEQ ID NO: 278 - RNA, 21 nt, synthetic construct
  source 1..21: mol_type = other RNA
tcagccgcaaggccataattt

SEQ ID NO: 279 - RNA, 21 nt, synthetic construct
  source 1..21: mol_type = other RNA
aactaaaattctcattcatct

SEQ ID NO: 280 - RNA, 21 nt, synthetic construct
  source 1..21: mol_type = other RNA
gtagctccggcttcaacccac

SEQ ID NO: 281 - RNA, 21 nt, synthetic construct
  source 1..21: mol_type = other RNA
agcctcctccactaaagtgtc

SEQ ID NO: 282 - RNA, 21 nt, synthetic construct
  source 1..21: mol_type = other RNA
ctccaagggtagctccggctt

SEQ ID NO: 283 - RNA, 21 nt, synthetic construct
  source 1..21: mol_type = other RNA
ttttgccatttgttaaataac

SEQ ID NO: 284 - RNA, 21 nt, synthetic construct
  source 1..21: mol_type = other RNA
cctacgccaacagtagggcaa

SEQ ID NO: 285 - RNA, 21 nt, synthetic construct
  source 1..21: mol_type = other RNA
tagattttcgatctagcactt

SEQ ID NO: 286 - RNA, 21 nt, synthetic construct
  source 1..21: mol_type = other RNA
tcaacgttgactaagtgtgca

SEQ ID NO: 287 - RNA, 21 nt, synthetic construct
  source 1..21: mol_type = other RNA
ttttgcatcaacgttgactaa

SEQ ID NO: 288 - RNA, 21 nt, synthetic construct
  source 1..21: mol_type = other RNA
taacagtagggcaatacccag

SEQ ID NO: 289 - RNA, 21 nt, synthetic construct
  source 1..21: mol_type = other RNA
tttgcatcaacgttgactaag

SEQ ID NO: 290 - RNA, 21 nt, synthetic construct
  source 1..21: mol_type = other RNA
atcaacgatattatcagccgc

SEQ ID NO: 291 - RNA, 21 nt, synthetic construct
  source 1..21: mol_type = other RNA
taattttgcatcaatggtcca

SEQ ID NO: 292 - RNA, 21 nt, synthetic construct
  source 1..21: mol_type = other RNA
aacgatattatcagccgcgag

SEQ ID NO: 293 - RNA, 21 nt, synthetic construct
  source 1..21: mol_type = other RNA
tgactaagtgtgcatcaacga

SEQ ID NO: 294 - RNA, 21 nt, synthetic construct
  source 1..21: mol_type = other RNA
taacccaataataaacttctc

SEQ ID NO: 295 - RNA, 21 nt, synthetic construct
  source 1..21: mol_type = other RNA
tgtccagctgcgctaacagta

SEQ ID NO: 296 - RNA, 21 nt, synthetic construct
  source 1..21: mol_type = other RNA
tcaatggtccatatcctcctc

SEQ ID NO: 297 - RNA, 21 nt, synthetic construct
  source 1..21: mol_type = other RNA
aattttgcatcaatggtccat

SEQ ID NO: 298 - RNA, 21 nt, synthetic construct
  source 1..21: mol_type = other RNA
atcaacgttgactaagtgtgc

SEQ ID NO: 299 - RNA, 21 nt, synthetic construct
  source 1..21: mol_type = other RNA
tagggcaatacccagcagcca

SEQ ID NO: 300 - RNA, 21 nt, synthetic construct
  source 1..21: mol_type = other RNA
caacgttgactaagtgtgcat

SEQ ID NO: 301 - RNA, 21 nt, synthetic construct
  source 1..21: mol_type = other RNA
atattatcagccgcgaggcca

SEQ ID NO: 302 - RNA, 21 nt, synthetic construct
  source 1..21: mol_type = other RNA
gactaagtgtgcatcaacgat

SEQ ID NO: 303 - RNA, 21 nt, synthetic construct
  source 1..21: mol_type = other RNA
atcaatggtccatatcctcct

SEQ ID NO: 304 - RNA, 21 nt, synthetic construct
  source 1..21: mol_type = other RNA
tattatcagccgcgaggccat

SEQ ID NO: 305 - RNA, 21 nt, synthetic construct
  source 1..21: mol_type = other RNA
tgcatcaatggtccatatcct

SEQ ID NO: 306 - RNA, 21 nt, synthetic construct
  source 1..21: mol_type = other RNA
caacgatattatcagccgcga

SEQ ID NO: 307 - RNA, 21 nt, synthetic construct
  source 1..21: mol_type = other RNA
tccaccaaagtgtccagctgc

SEQ ID NO: 308 - RNA, 21 nt, synthetic construct
  source 1..21: mol_type = other RNA
catcaacgttgactaagtgtg

SEQ ID NO: 309 - RNA, 21 nt, synthetic construct
  source 1..21: mol_type = other RNA
ttaacccaataataaacttct

SEQ ID NO: 310 - RNA, 21 nt, synthetic construct
  source 1..21: mol_type = other RNA
cagtagggcaatacccagcag

SEQ ID NO: 311 - RNA, 21 nt, synthetic construct
  source 1..21: mol_type = other RNA
ccaaagtgtccagctgcgcta

SEQ ID NO: 312 - RNA, 21 nt, synthetic construct
  source 1..21: mol_type = other RNA
ttatcagccgcgaggccataa

SEQ ID NO: 313 - RNA, 21 nt, synthetic construct
  source 1..21: mol_type = other RNA
tcctcctccaccaaagtgtcc

SEQ ID NO: 314 - RNA, 21 nt, synthetic construct
  source 1..21: mol_type = other RNA
ctagcacttttgcatcaacgt

SEQ ID NO: 315 - RNA, 21 nt, synthetic construct
  source 1..21: mol_type = other RNA
accaaagtgtccagctgcgct

SEQ ID NO: 316 - RNA, 21 nt, synthetic construct
  source 1..21: mol_type = other RNA
gtatagcccaaaagagatctt

SEQ ID NO: 317 - RNA, 21 nt, synthetic construct
  source 1..21: mol_type = other RNA
ataattttgcatcaatggtcc

SEQ ID NO: 318 - RNA, 21 nt, synthetic construct
  source 1..21: mol_type = other RNA
gaggccataattttgcatcaa

SEQ ID NO: 319 - RNA, 21 nt, synthetic construct
  source 1..21: mol_type = other RNA
tcagccgcgaggccataattt

SEQ ID NO: 320 - RNA, 21 nt, synthetic construct
  source 1..21: mol_type = other RNA
gatattatcagccgcgaggcc

SEQ ID NO: 321 - RNA, 21 nt, synthetic construct
  source 1..21: mol_type = other RNA
acagtagggcaatacccagca

SEQ ID NO: 322 - RNA, 21 nt, synthetic construct
  source 1..21: mol_type = other RNA
agggcaatacccagcagccaa

SEQ ID NO: 323 - DNA, 39 bp, synthetic construct
  misc_feature 1..39: note = THCAS cDNA forward primer
  source 1..39: mol_type = other DNA
accaggtctcaggagaactattttatgctctaagaaagt

SEQ ID NO: 324 - DNA, 37 bp, synthetic construct
  misc_feature 1..37: note = THCAS cDNA reverse primer
  source 1..37: mol_type = other DNA
accaggtctcatcgttcatgactcacttcataacaaa

SEQ ID NO: 325 - DNA, 37 bp, synthetic construct
  misc_feature 1..37: note = CBDAS-like-1 cDNA forward primer
  source 1..37: mol_type = other DNA
accaggtctcaggagaagtcccatttgttatagtaga

SEQ ID NO: 326 - DNA, 35 bp, synthetic construct
  misc_feature 1..35: note = CBDAS-like-1 cDNA forward primer
  source 1..35: mol_type = other DNA
accaggtctcatcgtgagatacatgagcttgtcaa

SEQ ID NO: 327 - DNA, 33 bp, synthetic construct
  misc_feature 1..33: note = CBCAS-2 cDNA forward primer
  source 1..33: mol_type = other DNA
accaggtctcaggagggccagtattctctgctc

SEQ ID NO: 328 - DNA, 36 bp, synthetic construct
  misc_feature 1..36: note = CBCAS-2 cDNA forward primer
  source 1..36: mol_type = other DNA
accaggtctcatcgtcacgactcacttcagaactag

SEQ ID NO: 329 - DNA, 29 bp, synthetic construct
  misc_feature 1..29: note = CBDAS-truncated-4 cDNA forward primer
  source 1..29: mol_type = other DNA
accaggtctcaggagccggagctaccctt

SEQ ID NO: 330 - DNA, 30 bp, synthetic construct
  misc_feature 1..30: note = CBDAS-truncated-4 cDNA forward primer
  source 1..30: mol_type = other DNA
accaggtctcatcgtggctatacgtggtgg

SEQ ID NO: 331 - DNA, 22 bp, synthetic construct
  misc_feature 1..22: note = Flanking Arm#1 forward primer
  source 1..22: mol_type = other DNA
catttggattgattacagttgg

SEQ ID NO: 332 - DNA, 18 bp, synthetic construct
  misc_feature 1..18: note = Flanking Arm#1 forward primer
  source 1..18: mol_type = other DNA
acccaacttaatcgcctt

SEQ ID NO: 333 - DNA, 20 bp, synthetic construct
  misc_feature 1..20: note = Flanking Arm#2 forward primer
  source 1..20: mol_type = other DNA
cgaatctcaagcaatcaagc

SEQ ID NO: 334 - DNA, 20 bp, synthetic construct
  misc_feature 1..20: note = Flanking Arm#2 forward primer
  source 1..20: mol_type = other DNA
attttgaggcatttcagtca

SEQ ID NO: 335 - DNA, 603 bp, synthetic construct
  misc_feature 1..603: note = THCAS cDNA insert
  source 1..603: mol_type = other DNA
aactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaa

SEQ ID NO: 336 - DNA, 442 bp, synthetic construct
  misc_feature 1..442: note = CBDAS-like-1 cDNA insert
  source 1..442: mol_type = other DNA
aagtcccatttgttatagtagacttgagaaacatgcattcggtcaaaatagatgttcatagccaaactgcatgggttgaagccggagctacccttggagaagtttattattggatcaatgagaacaatgagaatcttagttttcctgctgggtactgccctactgttggcgcgggtggacactttagtggaggaggctatggagcattgatgcgaaattatggcctcgcggctgataatatcattgatgcgcacttagtcaatgttgatggaaaagttttagatcgaaaatccatgggggaagatttgttttgggctatacgtggtggtggaggagaaaactttggaatcattgtagcgtggaaaattagacttgttgctgtcccatcaatgtctactatattcagtgttaaaaagaacatggagatacatgagcttgtcaa

SEQ ID NO: 337 - DNA, 606 bp, synthetic construct
  misc_feature 1..606: note = CBCAS-2 cDNA insert
  source 1..606: mol_type = other DNA
ggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaattcatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactag

SEQ ID NO: 338 - DNA, 247 bp, synthetic construct
  misc_feature 1..247: note = CBDAS-truncated-4 cDNA insert
  source 1..247: mol_type = other DNA
ccggagctacccttggagaagtttattattgggttaatgagaaaaatgagaatcttagtttggctgctgggtattgccctactgttagcgcagctggacactttggtggaggaggatatggaccattgatgcaaaattatggcctcgcggctgataatatcgttgatgcacacttagtcaacgttgatgcaaaagtgctagatcgaaaatctatgggggaagatctcttttgggctatacgtggtgg

SEQ ID NO: 339 - DNA, 22 bp, synthetic construct
  misc_feature 1..22: note = THCAS qPCR forward primer
  source 1..22: mol_type = other DNA
aatttttcatggtggagtggat

SEQ ID NO: 340 - DNA, 24 bp, synthetic construct
  misc_feature 1..24: note = THCAS qPCR forward primer
  source 1..24: mol_type = other DNA
aaaatttacaacaccactgtagaa

SEQ ID NO: 341 - DNA, 23 bp, synthetic construct
  misc_feature 1..23: note = CBDAS qPCR forward primer
  source 1..23: mol_type = other DNA
cagttacttctcctccattttcc

SEQ ID NO: 342 - DNA, 22 bp, synthetic construct
  misc_feature 1..22: note = CBDAS qPCR forward primer
  source 1..22: mol_type = other DNA
atagtatcaatccagctcaact

SEQ ID NO: 343 - DNA, 19 bp, synthetic construct
  misc_feature 1..19: note = CBCAS qPCR forward primer
  source 1..19: mol_type = other DNA
acctgggagaagcaagaag

SEQ ID NO: 344 - DNA, 24 bp, synthetic construct
  misc_feature 1..24: note = CBCAS qPCR forward primer
  source 1..24: mol_type = other DNA
ggattagtttttcctaaatcaagg

SEQ ID NO: 345 - DNA, 23 bp, synthetic construct
  misc_feature 1..23: note = primer
  source 1..23: mol_type = other DNA
ggaaaactgaagagaagtaagtg

SEQ ID NO: 346 - DNA, 22 bp, synthetic construct
  misc_feature 1..22: note = primer
  source 1..22: mol_type = other DNA
aattgagctggattgatactat